(19) United States
(12) Patent Application Publication
(10) Pub. No.: US 2022/0283500 A1
(43) Pub. Date: Sep. 8, 2022

(54) CHEMICALLY AMPLIFIED POSITIVE-TYPE PHOTOSENSITIVE RESIN COMPOSITION, PHOTOSENSITIVE DRY FILM, PRODUCTION METHOD FOR PHOTOSENSITIVE DRY FILM, PRODUCTION METHOD FOR PATTERNED RESIST FILM, COMPOUND, PHOTO-ACID GENERATOR, AND PRODUCTION METHOD FOR N-ORGANOSULFONYLOXY COMPOUND

(71) Applicant: TOKYO OHKA KOGYO CO., LTD., Kawasaki-shi (JP)

(72) Inventor: Akiya KAWAUE, Kawasaki-shi (JP)

(21) Appl. No.: 17/630,809

(22) PCT Filed: Jul. 30, 2020
(86) PCT No.: PCT/JP2020/029366

§ 371 Date: Jan. 27, 2022

(30) Foreign Application Priority Data
Aug. 2, 2019 (JP) 2019-143387
Aug. 2, 2019 (JP) 2019-143388

(51) Int. Cl. G03F 7/039; G03F 7/029; G03F 7/032; G03F 7/26; C07D 221/14

(52) U.S. Cl. CPC G03F 7/0392; G03F 7/0295; G03F 7/032; G03F 7/265; C07D 221/14

(57) ABSTRACT

A chemically amplified positive-type photosensitive resin composition in which the acid generator included has excellent solubility in a solvent and with which a resist pattern having excellent mask linearity is easily formed; a photosensitive dry film having a photosensitive layer formed from the composition; a method of manufacturing the photosensitive dry film; a method of manufacturing a patterned resist film using the composition; and a compound and an acid generator which can be added to the composition. The composition includes an acid generator which generates acid when irradiated with an active ray or radiation, and a resin whose solubility in alkali increases under action of an acid.

### TECHNICAL FIELD

The present invention relates to a chemically amplified positive-type photosensitive resin composition, a photosensitive dry film having a photosensitive layer formed from the chemically amplified positive-type photosensitive resin composition, a method of manufacturing the photosensitive dry film, a method of manufacturing a patterned resist film using the chemically amplified positive-type photosensitive resin composition, a novel compound, a novel photoacid generator, and a method of manufacturing a N-oragnosulfonyloxy compound.

### BACKGROUND ART

Photofabrication is now the mainstream of a microfabrication technique. The photofabrication is a generic name for techniques which are carried out by applying a photoresist composition to the surface of a processing target to form a photoresist layer, patterning this photoresist layer using photolithographic techniques, and then conducting chemical etching, electrolytic etching, or electroforming based mainly on electroplating, using the patterned photoresist layer (photoresist pattern) as a mask, to produce various types of precision parts, such as semiconductor packages, etc.

In recent years, high density packaging technologies have progressed in semiconductor packages along with downsizing of electronics devices, and the increase in package density has been developed on the basis of mounting multi-pin thin film in packages, miniaturization of package size, two-dimensional packaging technologies in flip-tip systems or three-dimensional packaging technologies. In these types of high density packaging techniques, connection terminals, for example, protruding electrodes (mounting terminals) known as bumps that protrude above the package or metal posts that connect redistribution lines (RDL) that extend from peripheral terminals on the wafer with the mounting terminals are disposed on the surface of the substrate with high precision.

In the photofabrication as described above, a photoresist composition is used, and chemically amplified photoresist compositions containing an acid generator have been known as such a photoresist composition (see Patent Documents 1 and 2 and the like). According to the chemically amplified photoresist composition, an acid is generated from the acid generator when irradiated with radiation (exposure) and diffusion of the acid is promoted through heat treatment, to cause an acid catalytic reaction with a base resin and the like in the composition resulting in a change to the alkali-solubility of the same.

Such a chemically amplified positive-type photoresist composition is used in forming plated articles such as a bump, a metal post, and Cu redistribution line, for example, by plating. Specifically, a photoresist layer having a desired film thickness is formed on a support such as a metal substrate using a chemically amplified photoresist composition, and the photoresist layer is exposed through a predetermined mask pattern and is developed, thereby forming a photoresist pattern to be used as a template in which portions for forming plated articles have been selectively removed (stripped). Then, bumps, metal posts, and Cu redistribution lines can be formed by embedding a conductor such as copper into the removed portions (nonresist portions) by plating, and then removing the surrounding photoresist pattern. Further, the chemically amplified positive-type photoresist composition is also used for forming an etching mask which is used, for example, to process a substrate by etching. Specifically, a photoresist layer having a desired film thickness is formed on a substrate by using the chemically amplified photoresist composition, and then only portions corresponding to etching target portions of the photoresist layer are exposed through a predetermined mask pattern, and then the exposed photoresist layer is developed to form a photoresist pattern used as an etching mask.
Patent Document 1: Japanese Unexamined Patent Application, Publication No. H09-176112
Patent Document 2: Japanese Unexamined Patent Application, Publication No. H11-52562

### DISCLOSURE OF THE INVENTION

### Problems to be Solved by the Invention

In order to faithfully produce a resist pattern with a desired dimension, it is important for the photoresist composition to have good mask fidelity (mask linearity), which can faithfully reproduce a mask pattern into the resist pattern. However, when a resist pattern is formed using a conventionally known chemically amplified positive photoresist composition as disclosed in Patent Document 1 or 2 and the like, it is often impossible to faithfully reproduce a mask pattern into the resist pattern.

In addition, the chemically amplified photoresist composition is often prepared by dissolving components such as an acid generator in a solvent such as propylene glycol monomethyl ether acetate, etc. Therefore, from the viewpoint of ease of preparation of the chemically amplified photoresist composition and prevention of precipitation of the acid generator during storage of the chemically amplified photoresist composition, it is desirable that the acid generator be excellent in solvent solubility.

The present invention has been made in view of the above-mentioned problems. An object of the present invention is to provide a chemically amplified positive-type photosensitive resin composition in which the acid generator included has excellent solubility in a solvent and with which a resist pattern having excellent mask linearity is easily formed, a photosensitive dry film having a photosensitive layer formed from the chemically amplified positive-type photosensitive resin composition, a method of manufacturing the photosensitive dry film, a method of manufacturing a patterned resist film using the chemically amplified positive-type photosensitive resin composition, and a compound and an acid generator which can be preferably added to the chemically amplified positive-type photosensitive resin composition. Furthermore, it is an object of the present invention to provide an efficient method for manufacturing a N-organosulfonyloxy compound which can be applied to the abovementioned method for manufacturing an acid generator.

### Means for Solving the Problems

As a result of extensive research to achieve the above objects, the present inventors have found that the above problems can be solved by a chemically amplified positive-type photosensitive resin composition which contains an acid generator (A) which generates acid upon exposure to an irradiated active ray or radiation and a resin (B) whose solubility in alkali increases under action of an acid, and in which the acid generator (A) contains a compound represented by the following formula (A1), thereby completing the present invention. Further, the present inventors have found that a N-organosulfonyloxy compound can be efficiently obtained by reacting a N-hydroxy compound (A′) with a sulfonyl fluoride compound (B′) in the presence of a basic compound (D′) while silylating the hydroxy group on the nitrogen atom possessed by the N-hydroxy compound (A′) with a silylating agent (C′), or silylating the hydroxy group on the nitrogen atom possessed by the N-hydroxy compound (A′) with a silylating agent (C′), and then reacting the silyated product generated with a sulfonyl fluoride (B′) in the presence of a basic compound (D′). Specifically, the present invention provides the following.

A first aspect of the present invention relates to a chemically amplified positive-type photosensitive resin composition including: an acid generator (A) which generates acid when irradiated with an active ray or radiation, and a resin (B) whose solubility in alkali increases under action of an acid. The acid generator (A) includes a compound represented by the following formula (A1):

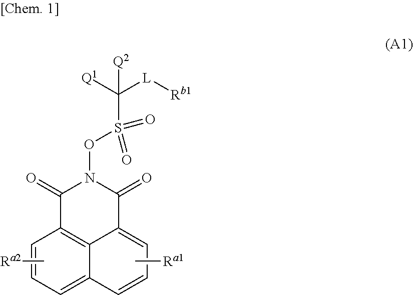

In the formula (A1), R^{b1} is a hydrocarbon group having 1 or more and 30 or less carbon atoms; when the hydrocarbon group as R^{b1} contains 1 or more methylene groups, at least a part of the methylene groups may be replaced with a group selected from the group consisting of —O—, —S—, —CO—, —CO—O—, —SO—, —SO₂—, —CR^{b4}R^{b5}—, and —NR^{b6}—; when the hydrocarbon group as R^{b1} contains a hydrocarbon ring, at least one of the carbon atoms constituting the hydrocarbon ring may be replaced with a heteroatom selected from the group consisting of N, O, P, S, and Se or an atomic group containing the heteroatom; R^{b4} and R^{b5} are each independently a hydrogen atom or a halogen atom, and at least one of R^{b4} and R^{b5} is a halogen atom; R^{b6} is a hydrogen atom or a hydrocarbon group having 1 or more and 6 or less carbon atoms; R^{a1} and R^{a2} are each independently a hydrogen atom, an optionally substituted aliphatic hydrocarbon group having 1 or more and 20 or less carbon atoms, an optionally substituted aromatic group having 5 or more and 20 or less ring constituting atoms, or a group represented by —R^{a3}—R^{a4}; R^{a1} and R^{a2} are not simultaneously hydrogen atoms; when the aliphatic hydrocarbon group as R^{a1} or R^{a2} contains 1 or more methylene groups, at least a part of the methylene groups may be replaced with a group selected from the group consisting of —O—, —S—, —CO—, —CO—O—, —SO—, —SO₂—, and —NR^{a5}—; R^{a5} is a hydrogen atom or a hydrocarbon group having 1 or more and 6 or less carbon atoms; R^{a3} is a methylene group, —O—, —CO—, —CO—O—, —SO—, —SO₂—, or —NR^{a6}—; R^{a6} is a hydrogen atom or a hydrocarbon group having 1 or more and 6 or less carbon atoms; R^{a4} is an optionally substituted aromatic group having 5 or more and 20 or less ring constituting atoms, a perfluoroalkyl group having 1 or more and 6 or less carbon atoms, an optionally substituted aralkyl group having 7 or more and 20 or less carbon atoms, or a heteroarylalkyl group having an optionally substituted aromatic heterocyclic group having 5 or more and 20 or less ring constituting atoms; Q¹ and Q² are each independently a fluorine atom or a perfluoroalkyl group having 1 or more and 6 or less carbon atoms; and L is an ester bond.

A second aspect of the present invention relates to a photosensitive dry film including a substrate film and a photosensitive layer formed on a surface of the substrate film, in which the photosensitive layer includes the chemically amplified positive-type photosensitive resin composition as described in the first aspect.

A third aspect of the present invention relates to a method of manufacturing a photosensitive dry film, the method including applying the chemically amplified positive-type photosensitive resin composition as described in the first aspect to a substrate film to form a photosensitive layer.

A fourth aspect of the present invention relates to a method of manufacturing a patterned resist film, the method including: laminating a photosensitive layer on a substrate, the photosensitive layer including the chemically amplified positive-type photosensitive resin composition as described in the first aspect; exposing the photosensitive layer through irradiation with an active ray or radiation in a position-selective manner; and developing the exposed photosensitive layer.

A fifth aspect of the present invention relates to a compound represented by the following formula (A1):

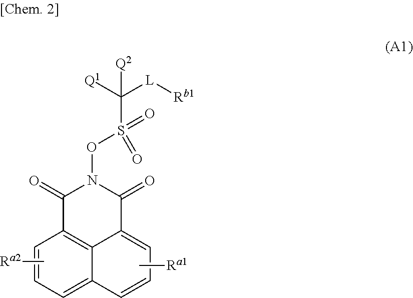

In the formula (A1), R^{b1} is a hydrocarbon group having 1 or more and 30 or less carbon atoms; when the hydrocarbon group as R^{b1} contains 1 or more methylene groups, at least a part of the methylene groups may be replaced with a group selected from the group consisting of —O—, —S—, —CO—, —CO—O—, —SO—, —SO₂—, —CR^{b4}R^{b5}—, and —NR^{b6}—; when the hydrocarbon group as R^{b1} contains a hydrocarbon ring, at least one of the carbon atoms constituting the hydrocarbon ring may be replaced with a heteroatom selected from the group consisting of N, O, P, S, and Se or an atomic group containing the heteroatom; R^{b4} and R^{b5} are each independently a hydrogen atom or a halogen atom, and at least one of R^{b4} and R^{b5} is a halogen atom; R^{b6} is a hydrogen atom or a hydrocarbon group having 1 or more and 6 or less carbon atoms; R^{a1} and R^{a2} are each independently a hydrogen atom, an optionally substituted aliphatic hydrocarbon group having 1 or more and 20 or less carbon atoms, an optionally substituted aromatic group having 5 or more and 20 or less ring constituting atoms, or a group represented by —R^{a3}—R^{a4}; R^{a1} and R^{a2} are not simultaneously hydrogen atoms; when the aliphatic hydrocarbon group as R^{a1} or R^{a2} contains 1 or more methylene groups, at least a part of the methylene groups may be replaced with a group selected from the group consisting of —O—, —S—, —CO—, —CO—O—, —SO—, —SO₂—, and —NR^{a5}—; R^{a5} is a hydrogen atom or a hydrocarbon group having 1 or more and 6 or less carbon atoms; Rai is a methylene group, —O—, —CO—, —CO—O—, —SO—, —SO₂—, or —NR^{a6}—; R^{a6} is a hydrogen atom or a hydrocarbon group having 1 or more and 6 or less carbon atoms; R^{a4} is an optionally substituted aromatic group having 5 or more and 20 or less ring constituting atoms, a perfluoroalkyl group having 1 or more and 6 or less carbon atoms, an optionally substituted aralkyl group having 7 or more and 20 or less carbon atoms, or a heteroarylalkyl group having an optionally substituted aromatic heterocyclic group having 5 or more and 20 or less ring constituting atoms; Q¹ and Q² are each independently a fluorine atom or a perfluoroalkyl group having 1 or more and 6 or less carbon atoms; and L is an ester bond.

A sixth aspect of the present invention relates to a photoacid generator including the compound as described in the fifth aspect.

A seventh aspect of the present invention relates to a method of manufacturing a N-organosulfonyloxy compound, the method including reacting a N-hydroxy compound (A′) and a sulfonyl fluoride compound (B′) in the presence of a basic compound (D′). The manufacturing method is characterized in that a silylating agent (C′) is present in the system while the N-hydroxy compound (A′) and the sulfonyl fluoride compound (B′) are reacted. The sulfonyl fluoride compound (B′) is represented by the following formula (bi1):

R^{b11}—SO₂—F (bi1)

(in formula (bi1), R^{b11} is an organic group), and the silylating agent (C′) is capable of converting a hydroxy group on the nitrogen atom possessed by the N-hydroxy compound (A′) into a silyloxy group represented by the following formula (ac1):

—O—Si(R^{c1})₃ (ac1)

(in the formula (ac1), each R^{c1} is independently a hydrocarbon group having 1 or more and 10 or less carbon atoms).

An eighth aspect of the present invention relates to a method of manufacturing a N-organosulfonyloxy compound, the method including silylating a N-hydroxy compound (A′) with a silylating agent (C′), and condensing a silylated product of the N-hydroxy compound (A′) generated in the silylation step with a sulfonyl fluoride compound (B′) in the presence of a basic compound (D′). The sulfonyl fluoride compound (B′) is represented by the following formula (bi1):

R^{b11}—SO₂—F (bi1)

(in formula (bi1), R^{b11} is an organic group), and the silylating agent is capable of converting a hydroxy group on the nitrogen atom possessed by the N-hydroxy compound (A′) into a silyloxy group represented by the following formula (ac1):

—O—Si(R^{c1})₃ (ac1)

(in formula (ac1), each R^{c1} is independently a hydrocarbon group having 1 or more and 10 or less carbon atoms).

### Effects of the Invention

The present invention can provide a chemically amplified positive-type photosensitive resin composition in which the acid generator included has excellent solubility in a solvent and with which a resist pattern having excellent mask linearity is easily formed, a photosensitive dry film having a photosensitive layer formed of the chemically amplified positive-type photosensitive resin composition, a method of manufacturing the photosensitive dry film, a method of manufacturing a patterned resist film using the chemically amplified positive-type photosensitive resin composition, and a compound and an acid generator which can be preferably added to the chemically amplified positive-type photosensitive resin composition. Further, according to the present invention, it is possible to provide a method of efficiently manufacturing a N-organosulfonyloxy compound which can be suitably used in the method of manufacturing an acid generator described above.

### PREFERRED MODE FOR CARRYING OUT THE INVENTION

### <<Chemically Amplified Positive-Type Photosensitive Resin Composition>>

The chemically amplified positive-type photosensitive resin composition (hereinafter, also referred to as a “photosensitive resin composition”) includes an acid generator (A) which generates acid upon exposure to an irradiated active ray or radiation (hereinafter also referred to as the acid generator (A)), and a resin (B) whose solubility in alkali increases under action of an acid (hereinafter also referred to as the resin (B)). In the present invention, the acid generator (A) includes a compound represented by the following formula (A1). The photosensitive resin composition may include components such as an alkali-soluble resin (D), a sulfur-containing compound (E), an acid diffusion suppressing agent (F), an organic solvent (S), and the like, as necessary.

The film thickness of the resist pattern formed using the photosensitive resin composition is not particularly limited, and can be applied to a thick film or a thin film. The photosensitive resin composition is preferably used for forming a resist pattern of a thick film. Specifically, the film thickness of the resist pattern formed using the photosensitive resin composition is preferably 0.5 μm or more, more preferably 0.5 μm or more and 300 μm or less, more preferably 0.5 μm or more and 200 μm or less, and most preferably 0.5 μm or more and 150 μm or less. The upper limit value of the film thickness may be, for example, 100 μm or less. The lower limit value of the film thickness may be, for example, 1 μm or more and may be 3 μm or more.

Hereinafter, described are essential or optional components in the photosensitive resin composition, and a method for manufacturing the photosensitive resin composition.

### <Acid Generator (A)>

The acid generator (A) is a compound which is capable of generating an acid when irradiated with an active ray or radiation, and which directly or indirectly generates an acid under the action of light. The acid generator (A) includes a compound represented by the following formula (A1):

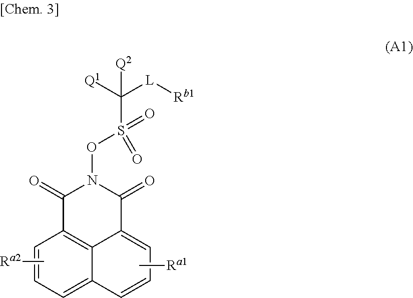

In the formula (A1), R^{b1} is a hydrocarbon group having 1 or more and 30 or less carbon atoms. When the hydrocarbon group as R^{b1} contains 1 or more methylene groups, at least a part of the methylene groups may be replaced with a group selected from the group consisting of —O—, —S—, —CO—, —CO—O—, —SO—, —SO₂—, —CR^{b4}R^{b5}—, and —NR^{b6}—. When the hydrocarbon group as R^{b1} contains a hydrocarbon ring, at least one of the carbon atoms constituting the hydrocarbon ring may be replaced with a heteroatom selected from the group consisting of N, O, P, S, and Se or an atomic group containing the heteroatom. R^{b4} and R^{b5} are each independently a hydrogen atom or a halogen atom, and at least one of R^{b4} and R^{b5} is a halogen atom. R^{b6} is a hydrogen atom or a hydrocarbon group having 1 or more and 6 or less carbon atoms. R^{a1} and R^{a2} are each independently a hydrogen atom, an optionally substituted aliphatic hydrocarbon group having 1 or more and 20 or less carbon atoms, an optionally substituted aromatic group having 5 or more and 20 or less ring constituting atoms, or a group represented by —R^{a3}—R^{a4}. R^{a1} and R^{a2} are not simultaneously hydrogen atoms. When the aliphatic hydrocarbon group as R^{a1} or R^{a2} contains 1 or more methylene groups, at least a part of the methylene groups may be replaced with a group selected from the group consisting of —O—, —S—, —CO—, —CO—O—, —SO—, —SO₂—, and —NR^{a5}—. R^{a5} is a hydrogen atom or a hydrocarbon group having 1 or more and 6 or less carbon atoms. Rai is a methylene group, —O—, —CO—, —CO—O—, —SO—, —SO₂—, or —NR^{a6}—. R^{a6} is a hydrogen atom or a hydrocarbon group having 1 or more and 6 or less carbon atoms. R^{a4} is an optionally substituted aromatic group having 5 or more and 20 or less ring constituting atoms, a perfluoroalkyl group having 1 or more and 6 or less carbon atoms, an optionally substituted aralkyl group having 7 or more and 20 or less carbon atoms, or a heteroarylalkyl group having an optionally substituted aromatic heterocyclic group having 5 or more and 20 or less ring constituting atoms. Q¹ and Q² are each independently a fluorine atom or a perfluoroalkyl group having 1 or more and 6 or less carbon atoms. L is an ester bond.

In the formula (A1), the aliphatic hydrocarbon group having 1 or more and 20 or less carbon atoms as R^{a1} and R^{a2} may be linear or branched, cyclic or a combination of these structures. As the aliphatic hydrocarbon group, an alkyl group is preferred. Suitable examples of the alkyl group include a methyl group, an ethyl group, a n-propyl group, an isopropyl group, a n-butyl group, an isobutyl group, a sec-butyl group, a tert-butyl group, a n-pentyl group, a n-hexyl group, a n-heptyl group, a n-octyl group, a 2-ethylhexyl group, a n-nonyl group, and a n-decyl group. Examples of the substituent which the aliphatic hydrocarbon group having 1 or more and 20 or less carbon atoms as R^{a1} and R^{a2} may have include a hydroxyl group, a mercapto group, an amino group, a halogen atom, an oxygen atom, a nitro group, a cyano group, a carboxy group, etc. The number of substituents is any number. Examples of the aliphatic hydrocarbon group as R^{a1} and R^{a2}, the aliphatic hydrocarbon group having 1 or more and 20 or less carbon atoms and having a substituent, include a perfluoroalkyl group having 1 or more and 6 or less carbon atoms. Examples thereof include CF₃—, CF₃CF₂—, (CF₃)₂CF—, CF₃CF₂CF₂—, CF₃CF₂CF₂CF₂—, (CF₃)₂CFCF₂—, CF₃CF₂ (CF₃) CF—, and (CF₃)₃C—.

In the formula (A1), an optionally substituted aromatic group having 5 or more and 20 or less ring constituting atoms as R^{a1} and R^{a2} may be an aromatic hydrocarbon group or an aromatic heterocyclic group. Examples of the aromatic group include aryl groups such as a phenyl group, a naphthyl group, etc. and heteroaryl groups such as a furyl group, a thienyl group, etc. Substituents which may be possessed by the aromatic group having 5 or more and 20 or less ring constituting atoms are the same as those which may be possessed by the aliphatic hydrocarbon group having 1 or more and 20 or less carbon atoms as R^{a1} and R^{a2}.

In the formula (A1), the optionally substituted aromatic group having 5 or more and 20 or less ring constituting atoms as R^{a4} is the same as the optionally substituted aromatic group having 5 or more and 20 or less ring constituting atoms described for R^{a1} and R^{a2}. In the formula (A1), the perfluoroalkyl group having 1 or more and 6 or less carbon atoms as R^{a4} is the same as the perfluoroalkyl group having 1 or more and 6 or less carbon atoms described as R^{a1} and R^{a2}. In the formula (A1), examples of the optionally substituted aralkyl group having 7 or more and 20 or less carbon atoms as R^{a4} include a benzyl group, a phenethyl group, an α-naphthylmethyl group, a β-naphthylmethyl group, a 2-α-naphthylethyl group, a 2-β-naphthylethyl group, etc. In the formula (A1), the heteroarylalkyl group is a group in which a part of the carbon atoms constituting an aromatic hydrocarbon ring in an arylalkyl group is substituted with a hetero atom such as N, O, S, or the like. Examples of the heteroarylalkyl group containing an optionally substituted aromatic heterocyclic group having ring configuration atoms of 5 or more and 20 or less as R^{a4} include a pyridine-2-ylmethyl group, a pyridine-3-ylmethyl group, a pyridine-4-ylmethyl group, and the like.

In the formula (A1), the hydrocarbon group having 1 or more and 6 or less carbon atoms as R^{a5} may be an aliphatic hydrocarbon group or an aromatic hydrocarbon group or a combination thereof. The aliphatic hydrocarbon group may be linear, branched, cyclic or a combination of these structures. Examples of the aliphatic hydrocarbon group include a methyl group, an ethyl group, a n-propyl group, an isopropyl group, a n-butyl group, an isobutyl group, a sec-butyl group, a tert-butyl group, a n-pentyl group, a n-hexyl group, etc. Examples of the aromatic hydrocarbon group include a phenyl group.

In the formula (A1), the hydrocarbon group having 1 or more and 6 or less carbon atoms as R^{a6} is the same as the hydrocarbon group having 1 or more and 6 or less carbon atoms described for R^{a5}.

In the formula (A1), the hydrocarbon group having 1 or more and 30 or less carbon atoms as R^{b1} may be an aliphatic hydrocarbon group or an aromatic hydrocarbon group or a combination thereof. The aliphatic hydrocarbon group may be linear, branched, cyclic or a combination of these structures. Examples of the aliphatic hydrocarbon group include a chain-like aliphatic hydrocarbon group such as a methyl group, an ethyl group, a n-propyl group, an isopropyl group, a n-butyl group, an isobutyl group, a sec-butyl group, a tert-butyl group, a n-pentyl group, a n-hexyl group, etc. and a cyclic aliphatic hydrocarbon group (hydrocarbon ring) such as a cyclopropyl group, a cyclobutyl group, a cyclopentyl group, a cyclohexyl group, an adamantyl group, a norbornyl group, etc. Examples of the aromatic hydrocarbon group include a phenyl group and a naphthyl group. Examples of the group in which an aliphatic hydrocarbon group and an aromatic hydrocarbon group are combined include a benzyl group, a phenethyl group, and a furylmethyl group. When the hydrocarbon group as R^{b1} contains a hydrocarbon ring, examples of the atomic group containing a hetero atom which replaces at least one of the carbon atoms which constitute the hydrocarbon ring include —CO—, —CO—O—, —SO—, —SO₂—, —SO₂—O—, —P(═O)—(OR^{b7})₃. R^{b7} is a hydrocarbon group having 1 or more and 6 or less carbon atoms, and is the same as the hydrocarbon group having 1 or more and 6 or less carbon atoms described for R^{a5}.

In the formula (A1), examples of halogen atoms as R^{b4} and R^{b5} include a chlorine atom, a fluorine atom, a bromine atom, and an iodine atom.

In the formula (A1), the hydrocarbon group having 1 or more and 6 or less carbon atoms as R^{b6} is the same as the hydrocarbon group having 1 or more and 6 or less carbon atoms described as R^{a5} in the formula (A1).

In the formula (A1), the perfluoroalkyl group having 1 or more and 6 or less carbon atoms as Q¹ and Q² is the same as the perfluoroalkyl group having 1 or more and 6 or less carbon atoms described as R^{a1} and R^{a2} in the formula (A1).

In the compound represented by the formula (A1), the direction of the ester bond as L is not particularly limited, and either —CO—O— or —O—CO— may be used.

The compound represented by the formula (A1) is preferably a compound represented by the following formula (A1-1) below.

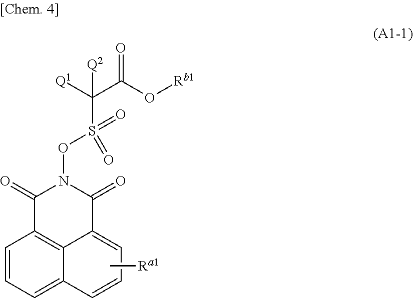

In the formula (A1-1), R^{b1}, R^{a1}, Q¹, and Q² are the same as those in the formula (A1).

When R^{a1} in the formula (A1-1) is an optionally substituted aliphatic hydrocarbon group having 1 or more and 20 or less carbon atoms and the aliphatic hydrocarbon group as R^{a1} contains 1 or more methylene groups, a compound represented by the formula (A1-1), in which at least a part of the methylene groups may be replaced with a group selected from the group consisting of —O—, —S—, —CO—, —CO—O—, —SO—, —SO₂—, and —NR^{a5}— is preferred.

Such a compound represented by the formula (A1) is excellent in solubility in a solvent such as propylene glycol monomethyl ether acetate. Therefore, it is easy to prepare a photosensitive resin composition containing the compound represented by the formula (A1) as the acid generator (A) at a desired concentration. Further, precipitation of the acid generator (A) in the prepared photosensitive resin composition is easily suppressed. Furthermore, when the photosensitive resin composition containing the compound represented by the formula (A1) as the acid generator (A) is used, a resist pattern excellent in mask linearity is easily formed. This is presumed to be due to the short diffusion length of acid generated by the compound represented by the formula (A1) upon exposure, and therefore, the acid does not easily diffuse into the unexposed portion. Incidentally, “mask linearity” means mask fidelity capable of faithfully reproducing the mask pattern into the resist pattern. Therefore, it is possible to realize a resist pattern having a desired dimension.

The compound represented by the formula (A1) can be produced by the following method of manufacturing a N-organosulfonyloxy compound. The method of manufacturing a N-organosulfonyloxy compound capable of producing a compound represented by the formula (A1) includes reacting a N-hydroxy compound (A′) and a sulfonyl fluoride compound (B′) in the presence of a basic compound (D′). The method is characterized in that while reacting the N-hydroxy compound (A′) with the sulfonyl fluoride compound (B′), a silylating agent (C′) is present in the system; the sulfonyl fluoride compound (B′) is represented by the following formula (b1-1); and the silylating agent (C′) is capable of converting a hydroxy group on the nitrogen atom possessed by the N-hydroxy compound (A′) into a silyloxy group represented by the following formula (ac1):

—O—Si(R^{c1})₃ (ac1)

(in formula (ac1), each R^{c1} is independently a hydrocarbon group having 1 or more and 10 or less carbon atoms),

R^{b1}-L-CQ¹Q²-SO₂—F (b1-1)

(in formula (b1-1), R^{b1}, L, Q¹, and Q² are the same as those in the above formula (A1), respectively).

In addition, the method of manufacturing a N-organosulfonyloxy compound represented by the formula (A1) includes silylating a N-hydroxy compound (A′) with a silylating agent (C′), and condensing a silylated product of the N-hydroxy compound (A′) produced in the silylating step with a sulfonyl fluoride compound (B′) in the presence of a basic compound (D′). The sulfonyl fluoride compound (B′) is represented by the above formula (b1-1), and the silylating agent is capable of converting a hydroxy group on the nitrogen atom possessed by the N-hydroxy compound (A′) into a silyloxy group represented by the above formula (ac1).

The N-hydroxy compound (A′) is a compound represented by the following formula (a1-1):

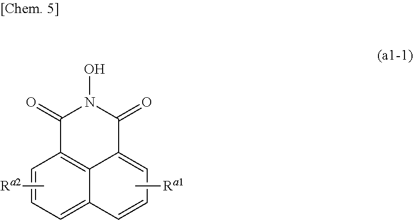

In the formula (a1-1), R^{a1} and R^{a2} are the same as those in the above formula (A1).

The N-hydroxy compound (A′) can be synthesized by conventional methods, for example, a method disclosed in the brochure of the PCT International Publication No. WO 2014/084269, Japanese Unexamined Patent Application (Translation of PCT Application), Publication No. 2017-535595, or the brochure of the PCT International Publication No. WO 2018/110399. For example, a compound represented by the formula (a1-1), in which R^{a2} is a hydrogen atom, can be synthesized by the reaction shown in the following formula using a commercially available bromide as a starting material. In the reaction, a bromo group on the naphthalic anhydride is converted into R^{a1}, and then the acid anhydride group is converted into a N-hydroxyimide by reacting with a hydroxylamine compound such as hydroxylamine hydrochloride, etc. As the N-hydroxy compound (A′), a commercially available product may be used.

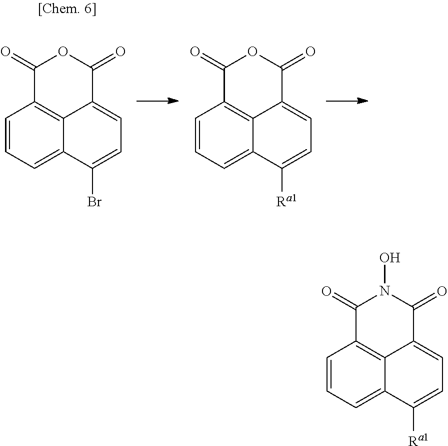

Furthermore, the N-hydroxy compound (A′) can also be synthesized by reacting the corresponding acenaphthoquinone with a peroxide in an organic solvent to obtain the corresponding 1,8-naphthalic anhydride and then reacting with the hydroxylamine compound. Furthermore, it can also be synthesized by reacting hydroxylamine with a precursor obtained by reacting 3-hydroxy-1,8-naphthalic anhydride with a compound of the following formula (i), or by reacting 4-bromo-hydroxy-1,8-naphthalic anhydride with a compound of the following formula (ii), or the like:

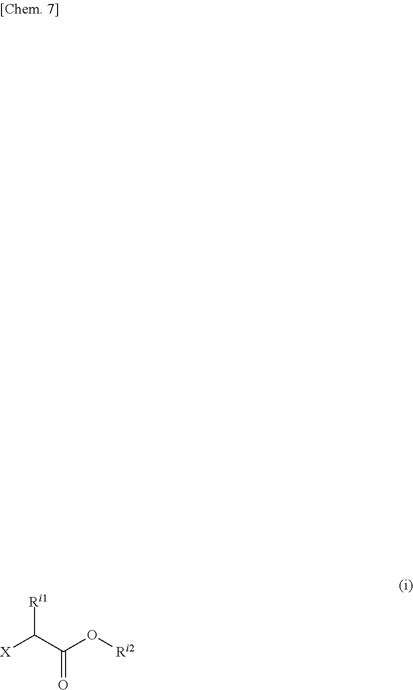

(in the formula (i), Rⁱ¹ represents a hydrogen atom, or an alkyl group which has 1 or more and 12 or less carbon atoms and which may have a carboxylic acid group, Rⁱ² represents a hydrogen atom, or an alkyl group having 1 or more and 12 or less carbon atoms, and Xⁱ represents a chlorine atom, a bromine atom, or an iodine atom), and

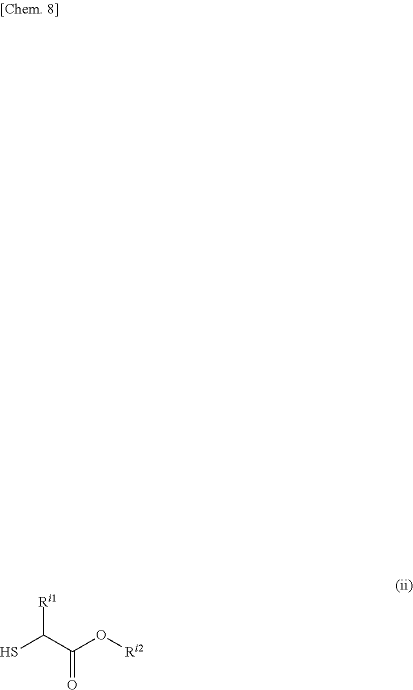

(in the formula (ii), Rⁱ¹ represents a hydrogen atom or an alkyl group which has 1 or more and 12 or less carbon atoms and which may have a carboxylic acid group, Rⁱ² represents a hydrogen atom or an alkyl group having 1 or more and 12 or less carbon atoms, and SH represents a thiol group).

The sulfonyl fluoride compound (B′) can be synthesized by conventional methods. For example, in formula (b1-1), a compound in which Q¹ and Q² are fluorine atoms can be synthesized by a reaction represented by the following formula. A commercially available product may be used as the sulfonyl fluoride compound (B′).

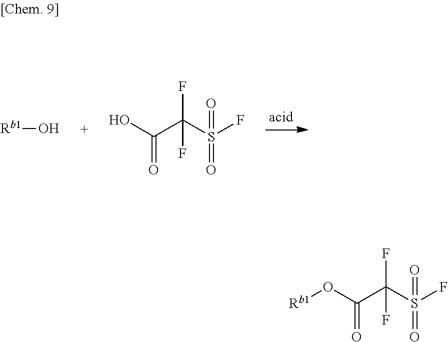

In formula (ac1), the hydrocarbon group having 1 or more and 10 or less carbon atoms as R^{c1} may be an aliphatic hydrocarbon group or an aromatic hydrocarbon group or a combination thereof. The aliphatic hydrocarbon group may be linear, branched, cyclic or a combination of these structures. Examples of the aliphatic hydrocarbon group include alkyl groups such as a methyl group, an ethyl group, a n-propyl group, an isopropyl group, a n-butyl group, an isobutyl group, a sec-butyl group, a tert-butyl group, a n-pentyl group, a n-hexyl group, a n-heptyl group, a n-octyl group, a 2-ethylhexyl group, a n-nonyl group, a n-decyl group, etc. Examples of the aromatic hydrocarbon group include a phenyl group and a naphthyl group.

Examples of the silylating agent (C′) include a compound represented by the following formula (c1):

X—Si(R^{c1})₃ (c1)

(in the formula (c1), R^{c1} is the same as R^{c1} in the formula (ac1), and X is a halogen atom).

In the formula (c1), examples of halogen atoms as X include a chlorine atom, a fluorine atom, a bromine atom, and an iodine atom.

Examples of the silylating agent (C′) include trimethylsilyl chloride, trimethylsilyl fluoride, trimethylsilyl bromide, t-butyldimethylsilyl chloride, ethyldimethylsilyl chloride, and isopropyldimethylsilyl chloride.

The basic compound (D′) may be an organic base or an inorganic base. Examples of the organic base include a nitrogen-containing basic compound, and examples thereof include amines such as methylamine, ethylamine, n-propylamine, isopropylamine, n-butylamine, dimethylamine, diethylamine, di-n-propylamine, diisopropylamine, di-n-butylamine, trimethylamine, triethylamine, methyldiethylamine, N-ethyldiisopropylamine, tri-n-isopropylamine, triisopropylamine, monoethanolamine, diethanolamine, triethanolamine, etc.; cyclic basic compounds such as pyrrole, piperidine, 1,8-diazabicyclo[5,4,0]-7-undecene, 1,5-diazabicyclo[4,3,0]-5-nonane, etc.; quaternary ammonium salts such as tetramethylammonium hydroxide (TMAH), tetraethylammonium hydroxide, tetrapropylammonium hydroxide (TPAH), tetrabutylammonium hydroxide, methyltripropylammonium hydroxide, methyltributylammonium hydroxide, benzyltrimethylammonium hydroxide, benzyltriethylammonium hydroxide, trimethyl (2-hydroxyethyl)ammonium hydroxide, etc., and the like. Examples of the inorganic base include metal hydroxides, metal hydrogen carbonates, metal bicarbonates, etc. Examples of the inorganic base include metal hydroxides such as lithium hydroxide, potassium hydroxide, sodium hydroxide, rubidium hydroxide, cesium hydroxide, magnesium hydroxide, calcium hydroxide, strontium hydroxide, barium hydroxide, etc.; metal carbonates such as lithium carbonate, potassium carbonate, sodium carbonate, rubidium carbonate, cesium carbonate, magnesium carbonate, calcium carbonate, strontium carbonate, barium carbonate, etc.; metal bicarbonates such as lithium bicarbonate, potassium bicarbonate, sodium bicarbonate, sodium bicarbonate, rubidium bicarbonate, cesium bicarbonate, etc. and the like.

In the method of manufacturing a N-organosulfonyloxy compound, the N-hydroxy compound (A′) and the sulfonyl fluoride compound (B′) as described above are reacted in the presence of the silylating agent (C′) and the basic compound (D′). As described above, when the N-hydroxy compound (A′) and the sulfonyl fluoride compound (B′) are reacted in the presence of the basic compound (D′), presence of the silylating agent (C′) enables efficient production of the N-organosulfonyloxy compound as shown in the Examples to be described below. For example, the N-organosulfonyloxy compound can be obtained at a yield of 65% or more with respect to the N-hydroxy compound (A′) and the sulfonyl fluoride compound (B′) as the raw materials.

By the method of manufacturing a N-organosulfonyloxy compound, a N-organosulfonyloxy compound having a structure in which a group obtained by removing a hydrogen atom of a hydroxy group bonded to the nitrogen atom of the N-hydroxy compound (A′) and R^{b1}—SO₂— derived from the sulfonyl fluoride compound (B′) are bonded to each other is obtained.

In the method of manufacturing a N-organosulfonyloxy compound, when the N-hydroxy compound (A′) and the sulfonyl fluoride compound (B′) are reacted in the presence of the basic compound (D′), it is sufficient for the silylating agent (C′) to exist in the reaction system. The N-hydroxy compound (A′), the sulfonyl fluoride compound (B′), the silylating agent (C′) and the basic compound (D′) may be mixed simultaneously or after the N-hydroxy compound (A′) and the silylating agent (C′) are partially or completely reacted, the sulfonyl fluoride (B′) and the basic compound (D′) may be added.

As described above, when the N-hydroxy compound (A′) and sulfonyl fluoride compound (B′) are reacted in the presence of the silylating agent (C′) and the basic compound (D′), the N-hydroxy compound (A′) is silylated by the silylating agent (C′) and the hydroxy group on the nitrogen atom is converted into a silyloxy group represented by the above formula (ac1) (Step 1: silylation step). Then, the silylated product of the N-hydroxy compound (A′) generated in the silylation step is condensed with the sulfonyl fluoride compound (B′) to which the basic compound (D′) has acted (Step 2: condensation step). This gives the N-organosulfonyloxy compound.

As an example of the method of manufacturing a N-organosulfonyloxy compound, a reaction formula is shown below, in which a compound represented by the formula (a1-1) is used as the N-hydroxy compound (A′), a compound represented by the formula (b1-1), in which Q¹ and Q² are fluorine atoms, is used as the sulfonyl fluoride compound (B′), trimethylsilyl chloride is used as the silylating agent (C′), and triethylamine is used as the basic compound (D′). Note that the reaction mechanism shown below is not an analytically confirmed reaction mechanism, but a reaction mechanism estimated from raw materials and behaviors thereof during the reaction.

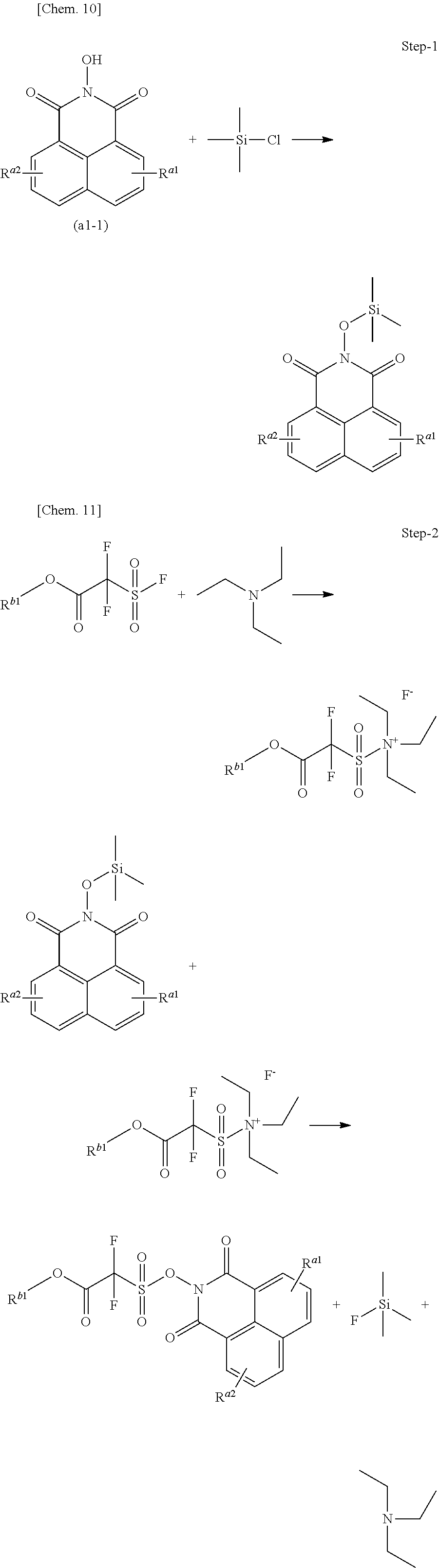

Examples of organic solvents which can be adopted for the reaction include: esters, such as ethyl acetate, butyl acetate, cellosolve acetate, and the like, ketones such as methyl ethyl ketone, isobutyl ketone, and methyl isobutyl ketone, etc., esters such as ethyl acetate, butyl acetate, diethyl malonate, etc., amides such as N-methylpyrrolidone, N,N-dimethylformamide, etc., ethers such as diethyl ether, ethyl cyclopentyl ether, tetrahydrofuran, dioxane, etc., aromatic hydrocarbons such as toluene, xylene, etc., aliphatic hydrocarbons such as hexane, heptane, octane, decahydronaphthalene, etc., halogenated hydrocarbons such as chloroform, dichloromethane, methylene chloride, ethylene chloride, etc., nitrile-based solvents such as acetonitrile, propionitrile, etc., dimethyl sulfoxide, dimethyl sulfamide, and the like. With respect to the organic solvents to be used, one type may be used alone, or two or more types may be combined in any manner and used. The reaction temperature which can be employed is, for example, in the range of −10° C. to 200° C., preferably in the range of 0° C. to 150° C., and more preferably in the range of 5° C. to 120° C. The reaction time which can be employed is, for example, 5 minutes or more and 20 hours or less, 10 minutes or more and 15 hours or less, or 30 minutes or more and 12 hours or less.

It is preferable to use each of the sulfonyl fluoride compound (B′), the silylating agent (C′) and the basic compound (D′) in an excessive amount, with respect to the N-hydroxy compound (A′). For example, it is preferable to use the sulfonyl fluoride compound (B′) in an amount of 1.1 mol or more and 2.5 mol or less, the silylating agent (C′) in an amount of 1.1 mol or more and 2.5 mol or less, and the basic compound (D′) in an amount of 1.1 mol or more and 2.5 mol or less, with respective to 1.0 mol of the N-hydroxy compound (A′).

The acid generator (A) may include another acid generator (hereinafter, also referred to as the another acid generator) other than the compound represented by the above formula (A1). The another acid generator is a compound which generates acid when irradiated with an active ray or radiation, and which generates acid by the light directly or indirectly. As the another acid generator which the acid generator (A) may include, the acid generator of the first to fifth aspect mentioned below is preferable.

The first aspect of the another acid generator in the acid generator (A) may be a compound represented by the following formula (a101).

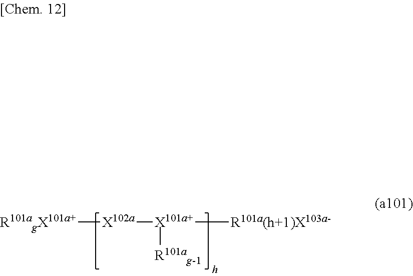

In the formula (a101), X^{101a} represents a sulfur atom or iodine atom having a valence of g; g represents 1 or 2. h represents the number of repeating units in the structure within parentheses. R^{101a} represents an organic group that is bonded to X^{101a}, and represents an aryl group having 6 or more and 30 or less carbon atoms, a heterocyclic group having 4 or more and 30 or less carbon atoms, an alkyl group having 1 or more and 30 or less carbon atoms, an alkenyl group having 2 or more and 30 or less carbon atoms, or an alkynyl group having 2 or more and 30 or less carbon atoms, and R^{101a} may be substituted with at least one selected from the group consisting of an alkyl group, a hydroxyl group, an alkoxy group, an alkylcarbonyl group, an arylcarbonyl group, an alkoxycarbonyl group, an aryloxycarbonyl group, an arylthiocarbonyl group, an acyloxy group, an arylthio group, an alkylthio group, an aryl group, a heterocyclic group, an aryloxy group, an alkylsulfinyl group, an arylsulfinyl group, an alkylsulfonyl group, an arylsulfonyl group, an alkyleneoxy group, an amino group, a cyano group, a nitro group, and halogen atoms. The number of R^{101a}s is g+h(g−1)+1, and the R^{101a}s may be respectively identical to or different from each other. Furthermore, two or more R^{101a}s may be bonded to each other directly or via —O—, —S—, —SO—, —SO₂—, —NH—, —NR^{102a}—, —CO—, —COO—, —CONH—, an alkylene group having 1 or more and 3 or less carbon atoms, or a phenylene group, and may form a ring structure including X^{101a}. R^{102a} represents an alkyl group having 1 or more and 5 or less carbon atoms, or an aryl group having 6 or more and 10 or less carbon atoms.

X^{102a} represents a structure represented by the following formula (a102).

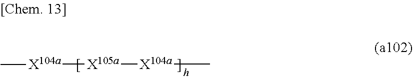

In the above formula (a102), X^{104a} represents an alkylene group having 1 or more and 8 or less carbon atoms, an arylene group having 6 or more and 20 or less carbon atoms, or a divalent group of a heterocyclic compound having 8 or more and 20 or less carbon atoms, and X^{104a} may be substituted with at least one selected from the group consisting of an alkyl group having 1 or more and 8 or less carbon atoms, an alkoxy group having 1 or more and 8 or less carbon atoms, an aryl group having 6 or more and 10 or less carbon atoms, a hydroxyl group, a cyano group, a nitro group, and halogen atoms. X^{105a} represents —O—, —S—, —SO—, —SO₂—, —NH—, —NR^{102a}—, —CO—, —COO—, —CONH—, an alkylene group having 1 or more and 3 or less carbon atoms, or a phenylene group. h represents the number of repeating units of the structure in parentheses. X^{104a}s in the number of h+1 and X^{105a}s in the number of h may be identical to or different from each other. R^{102a} has the same definition as described above.

X^{103a-} represents a counter ion of an onium, and examples thereof include a fluorinated alkylfluorophosphoric acid anion represented by the following formula (a117) or a borate anion represented by the following formula (a118).

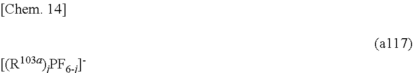

In the formula (a117), R^{103a} represents an alkyl group having 80% or more of the hydrogen atoms substituted with fluorine atoms. j represents the number of R^{103a}s and is an integer of 1 or more and 5 or less. R^{103a}s in the number of j may be respectively identical to or different from each other.

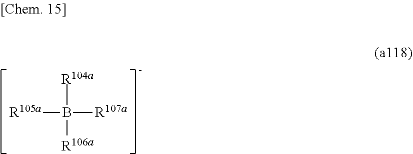

In the formula (a118) R^{104a} to R^{107a} each independently represents a fluorine atom or a phenyl group, and a part or all of the hydrogen atoms of the phenyl group may be substituted with at least one selected from the group consisting of a fluorine atom and a trifluoromethyl group.

Examples of the onium ion in the compound represented by the above formula (a101) include triphenylsulfonium, tri-p-tolylsulfonium, 4-(phenylthio)phenyldiphenylsulfonium, bis[4-(diphenylsulfonio)phenyl] sulfide, bis[4-{bis[4-(2-hydroxyethoxy)phenyl]sulfonio]phenyl} sulfide, bis{4-[bis(4-fluorophenyl)sulfonio]phenyl} sulfide, 4-(4-benzoyl-2-chlorophenylthio)phenylbis(4-fluorophenyl)sulfonium, 7-isopropyl-9-oxo-10-thia-9,10-dihydroanthracen-2-yldi-p-tolylsulfonium, 7-isopropyl-9-oxo-10-thia-9,10-dihydroanthracen-2-yldiphenylsulfonium, 2-[(diphenyl)sulfonio]thioxanthone, 4-[4-(4-tert-butylbenzoyl)phenylthio]phenyldi-p-tolylsulfonium, 4-(4-benzoylphenylthio)phenyldiphenylsulfonium, diphenylphenacylsulfonium, 4-hydroxyphenylmethylbenzylsulfonium, 2-naphthylmethyl(1-ethoxycarbonyl)ethylsulfonium, 4-hydroxyphenylmethylphenacylsulfonium, phenyl[4-(4-biphenylthio)phenyl]4-biphenylsulfonium, phenyl[4-(4-biphenylthio)phenyl]3-biphenylsulfonium, [4-(4-acetophenylthio)phenyl]diphenylsulfonium, octadecylmethylphenacylsulfonium, diphenyliodonium, di-p-tolyliodonium, bis(4-dodecylphenyl)iodonium, bis(4-methoxyphenyl)iodonium, (4-octyloxyphenyl)phenyliodonium, bis(4-decyloxy)phenyliodonium, 4-(2-hydroxytetradecyloxy)phenylphenyliodonium, 4-isopropylphenyl(p-tolyl)iodonium, 4-isobutylphenyl(p-tolyl)iodonium, or the like.

Among the onium ions in the compound represented by the above formula (a101), a preferred onium ion may be a sulfonium ion represented by the following formula (a119).

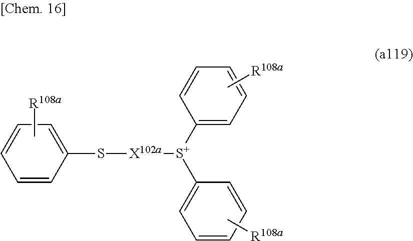

In the above formula (a119), R^{108a}s each independently represents a hydrogen atom or a group selected from the group consisting of alkyl, hydroxyl, alkoxy, alkylcarbonyl, alkylcarbonyloxy, alkyloxycarbonyl, a halogen atom, an aryl which may have a substituent, and arylcarbonyl. X^{102a} has the same definition as X^{102a} in the above formula (a101).

Specific examples of the sulfonium ion represented by the above formula (a119) include 4-(phenylthio)phenyldiphenylsulfonium, 4-(4-benzoyl-2-chlorophenylthio)phenylbis(4-fluorophenyl)sulfonium, 4-(4-benzoylphenylthio)phenyldiphenylsulfonium, phenyl[4-(4-biphenylthio)phenyl]4-biphenylsulfonium, phenyl[4-(4-biphenylthio)phenyl]3-biphenylsulfonium, [4-(4-acetophenylthio)phenyl]diphenylsulfonium, and diphenyl[4-(p-terphenylthio)phenyl]diphenylsulfonium.

In regard to the fluorinated alkylfluorophosphoric acid anion represented by the above formula (a117), R^{103a} represents an alkyl group substituted with a fluorine atom, and a preferred number of carbon atoms is 1 or more and 8 or less, while a more preferred number of carbon atoms is 1 or more and 4 or less. Specific examples of the alkyl group include linear alkyl groups such as methyl, ethyl, propyl, butyl, pentyl and octyl; branched alkyl groups such as isopropyl, isobutyl, sec-butyl and tert-butyl; and cycloalkyl groups such as cyclopropyl, cyclobutyl, cyclopentyl, and cyclohexyl. The proportion of hydrogen atoms substituted with fluorine atoms in the alkyl groups is usually 80% or more, preferably 90% or more, and even more preferably 100%. If the substitution ratio of fluorine atoms is less than 80%, the acid strength of the onium fluorinated alkylfluorophosphate represented by the above formula (a101) decreases.

A particularly preferred example of R^{103a} is a linear or branched perfluoroalkyl group having 1 or more and 4 or less carbon atoms and a substitution ratio of fluorine atoms of 100%. Specific examples thereof include CF₃, CF₃CF₂, (CF₃)₂CF, CF₃CF₂CF₂, CF₃CF₂CF₂CF₂, (CF₃)₂CFCF₂, CF₃CF₂ (CF₃) CF, and (CF₃)₃C. j which is the number of R^{103a}s represents an integer of 1 or more and 5 or less, and is preferably 2 or more and 4 or less, and particularly preferably 2 or 3.

Preferred specific examples of the fluorinated alkylfluorophosphoric acid anion include [(CF₃CF₂)₂PF₄]⁻, [(CF₃CF₂)₃PF₃]⁻, [((CF₃)₂CF)₂PF₄]⁻, [((CF₃)₂CF)₃PF₃]⁻, [(CF₃CF₂CF₂)₂PF₄]⁻, [(CF₃CF₂CF₂)₃PF₃]⁻, [((CF₃)₂CF CF₂)₂PF₄]⁻, [((CF₃)₂CF CF₂)₃PF₃]⁻, [(CF₃CF₂CF₂CF₂)₂PF₄]⁻, or [(CF₃CF₂CF₂)₃PF₃]⁻. Among these, [(CF₃CF₂)₃PF₃]⁻, [(CF₃CF₂CF₂)₃PF₃]⁻, [((CF₃)₂CF)₃PF₃]⁻, [((CF₃)₂CF)₂PF₄]⁻, [((CF₃)₂CF CF₂)₃PF₃]⁻, or [((CF₃)₂CFCF₂)₂PF₄]⁻ are particularly preferred.

Preferred specific examples of the borate anion represented by the above formula (a118) include tetrakis(pentafluorophenyl)borate ([B(C₆F₅)₄]⁻), tetrakis[(trifluoromethyl)phenyl]borate ([B(C₆H₄CF₃)₄]⁻), difluorobis(pentafluorophenyl)borate ([(C₆F₅)₂BF₂]⁻), trifluoro(pentafluorophenyl)borate ([(C₆F₅)BF₃]⁻), and tetrakis(difluorophenyl)borate ([B(C₆H₃F₂)₄]⁻). Among these, tetrakis(pentafluorophenyl)borate ([B(C₆F₅)₄]⁻) is particularly preferred.

The second aspect of the other acid generator in the acid generator (A) include halogen-containing triazine compounds such as 2,4-bis(trichloromethyl)-6-piperonyl-1,3,5-triazine, 2,4-bis(trichloromethyl)-6-[2-(2-furyl)ethenyl]-s-triazine, 2,4-bis(trichloromethyl)-6-[2-(5-methyl-2-furyl)ethenyl]-s-triazine, 2,4-bis(trichloromethyl)-6-[2-(5-ethyl-2-furyl)ethenyl]-s-triazine, 2,4-bis(trichloromethyl)-6-[2-(5-propyl-2-furyl)ethenyl]-s-triazine, 2,4-bis(trichloromethyl)-6-[2-(3,5-dimethoxyphenyl)ethenyl]-s-triazine, 2,4-bis(trichloromethyl)-6-[2-(3,5-diethoxyphenyl)ethenyl]-s-triazine, 2,4-bis(trichloromethyl)-6-[2-(3,5-dipropoxyphenyl)ethenyl]-s-triazine, 2,4-bis(trichloromethyl)-6-[2-(3-methoxy-5-ethoxyphenyl)ethenyl]-s-triazine, 2,4-bis(trichloromethyl)-6-[2-(3-methoxy-5-propoxyphenyl)ethenyl]-s-triazine, 2,4-bis(trichloromethyl)-6-[2-(3,4-methylenedioxyphenyl)ethenyl]-s-triazine, 2,4-bis(trichloromethyl)-6-(3,4-methylenedioxyphenyl)-s-triazine, 2,4-bis-trichloromethyl-6-(3-bromo-4-methoxy)phenyl-s-triazine, 2,4-bis-trichloromethyl-6-(2-bromo-4-methoxy)phenyl-s-triazine, 2,4-bis-trichloromethyl-6-(2-bromo-4-methoxy)styrylphenyl-s-triazine, 2,4-bis-trichloromethyl-6-(3-bromo-4-methoxy)styrylphenyl-s-triazine, 2-(4-methoxyphenyl)-4,6-bis(trichloromethyl)-1,3,5-triazine, 2-(4-methoxynaphthyl)-4,6-bis(trichloromethyl)-1,3,5-triazine, 2-[2-(2-furyl)ethenyl]-4,6-bis(trichloromethyl)-1,3,5-triazine, 2-[2-(5-methyl-2-furyl)ethenyl]-4,6-bis(trichloromethyl)-1,3,5-triazine, 2-[2-(3,5-dimethoxyphenyl)ethenyl]-4,6-bis(trichloromethyl)-1,3,5-triazine, 2-[2-(3,4-dimethoxyphenyl)ethenyl]-4,6-bis(trichloromethyl)-1,3,5-triazine, 2-(3,4-methylenedioxyphenyl)-4,6-bis(trichloromethyl)-1,3,5-triazine, tris(1,3-dibromopropyl)-1,3,5-triazine and tris(2,3-dibromopropyl)-1,3,5-triazine, and halogen-containing triazine compounds represented by the following formula (a103) such as tris(2,3-dibromopropyl)isocyanurate.

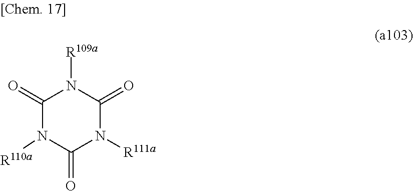

In the above formula (a103), R^{109a}, R^{110a} and R^{111a} each independently represent a halogenated alkyl group.

Further, the third aspect of the other acid generator of the acid generator (A) include α-(p-toluenesulfonyloxyimino)-phenylacetonitrile, α-(benzenesulfonyloxyimino)-2,4-dichlorophenylacetonitrile, α-(benzenesulfonyloxyimino)-2,6-dichlorophenylacetonitrile, α-(2-chlorobenzenesulfonyloxyimino)-4-methoxyphenylacetonitrile and α-(ethylsulfonyloxyimino)-1-cyclopentenylacetonitrile, and compounds represented by the following formula (a104) having an oximesulfonate group.

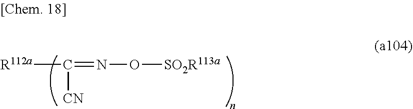

In the above formula (a104), R^{112a} represents a monovalent, bivalent or trivalent organic group, R^{113a} represents a substituted or unsubstituted saturated hydrocarbon group, an unsaturated hydrocarbon group, or an aromatic group, and n represents the number of repeating units of the structure in the parentheses.

In the formula (a104), examples of the aromatic group include aryl groups such as a phenyl group and a naphthyl group, and heteroaryl groups such as a furyl group and a thienyl group. These may have one or more appropriate substituents such as halogen atoms, alkyl groups, alkoxy groups and nitro groups on the rings. It is particularly preferable that R^{113a} is an alkyl group having 1 or more and 6 or less carbon atoms such as a methyl group, an ethyl group, a propyl group, and a butyl group. In particular, compounds in which R^{112a} represents an aromatic group, and R^{113a} represents an alkyl group having 1 or more and 4 or less carbon atoms are preferred.

Examples of the acid generator represented by the above formula (a104) include compounds in which R^{112a} is any one of a phenyl group, a methylphenyl group and a methoxyphenyl group, and R^{113a} is a methyl group, provided that n is 1, and specific examples thereof include α-(methylsulfonyloxyimino)-1-phenylacetonitrile, α-(methylsulfonyloxyimino)-1-(p-methylphenyl)acetonitrile, α-(methylsulfonyloxyimino)-1-(p-methoxyphenyl)acetonitrile, [2-(propylsulfonyloxyimino)-2,3-dihydroxythiophene-3-ylidene] (o-tolyl) acetonitrile and the like. Provided that n is 2, the acid generator represented by the above formula (a104) is specifically an acid generator represented by the following formulae.

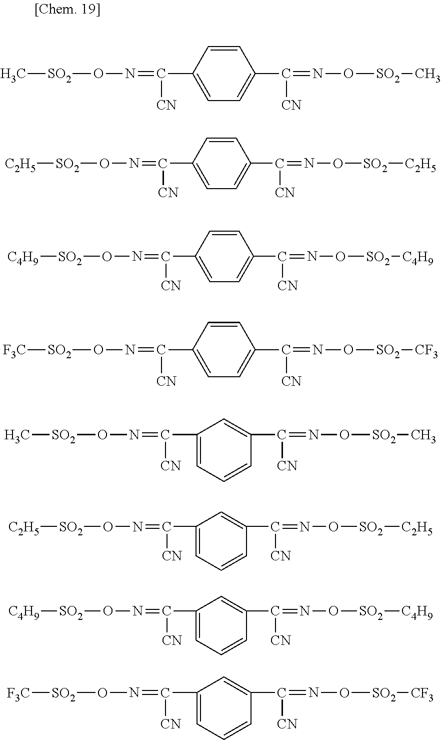

In addition, the other acid generators of the fourth aspect of the acid generator (A) include onium salts that have a naphthalene ring at their cation moiety. The expression “have a naphthalene ring” indicates having a structure derived from naphthalene and also indicates at least two ring structures and their aromatic properties are maintained. The naphthalene ring may have a substituent such as a linear or branched alkyl group having 1 or more and 6 or less carbon atoms, a hydroxyl group, a linear or branched alkoxy group having 1 or more and 6 or less carbon atoms or the like. The structure derived from the naphthalene ring, which may be of a monovalent group (one free valence) or of a bivalent group (two free valences), is desirably of a monovalent group (in this regard, the number of free valence is counted except for the portions connecting with the substituents described above). The number of naphthalene rings is preferably 1 or more and 3 or less.

Preferably, the cation moiety of the onium salt having a naphthalene ring at the cation moiety is of the structure represented by the following formula (a105).

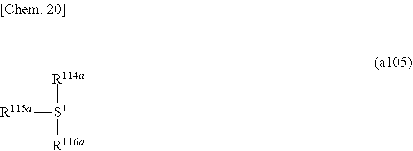

In the above formula (a105), at least one of R^{114a}, R^{115a} and R^{116a} represents a group represented by the following formula (a106), and the remaining represents a linear or branched alkyl group having 1 or more and 6 or less carbon atoms, a phenyl group which may have a substituent, a hydroxyl group, or a linear or branched alkoxy group having 1 or more and 6 or less carbon atoms. Alternatively, one of R^{114a}, R^{115a} and R^{116a} is a group represented by the following formula (a106), and the remaining two are each independently a linear or branched alkylene group having 1 or more and 6 or less carbon atoms, and these terminals may bond to form a ring structure.

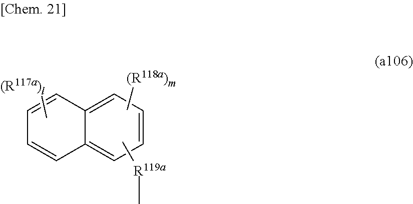

In the formula (a106), R^{117a} and R^{118a} each independently represent a hydroxyl group, a linear or branched alkoxy group having 1 or more and 6 or less carbon atoms, or a linear or branched alkyl group having 1 or more and 6 or less carbon atoms, and R^{119a} represents a single bond or a linear or branched alkylene group having 1 or more and 6 or less carbon atoms that may have a substituent. 1 and m each independently represent an integer of 0 or more and 2 or less, and 1+m is 3 or less. Herein, when there exists a plurality of R^{117a}, they may be identical to or different from each other. Furthermore, when there exists a plurality of R^{118a}, they may be identical to or different from each other.

Preferably, among R^{114a}, R^{115a} and R^{116a} as above, the number of groups represented by the above formula (a106) is one in view of the stability of the compound, and the remaining are linear or branched alkylene groups having 1 or more and 6 or less carbon atoms of which the terminals may bond to form a ring. In this case, the two alkylene groups described above form a 3 to 9 membered ring including sulfur atom(s). Preferably, the number of atoms to form the ring (including sulfur atom(s)) is 5 or more and 6 or less.

Examples of the substituent, which the alkylene group may have, include an oxygen atom (in this case, a carbonyl group is formed together with a carbon atom that constitutes the alkylene group), a hydroxyl group or the like.

Furthermore, examples of the substituent, which the phenyl group may have, include a hydroxyl group, a linear or branched alkoxy group having 1 or more and 6 or less carbon atoms, a linear or branched alkyl group having 1 or more and 6 or less carbon atoms, or the like.

Examples of cations for the suitable cation moiety include cations represented by the following formulae (a107) and (a108), and the structure represented by the following formula (a108) is particularly preferable.

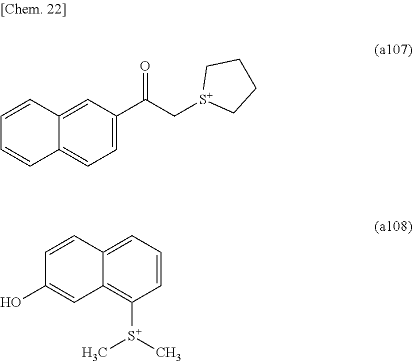 [SMILES: O=C(C[S+]1CCCC1)c1ccc2ccccc2c1 ; C[S+](C)c1cccc2ccc(O)cc12]

The cation moieties, which may be of an iodonium salt or a sulfonium salt, are desirably of a sulfonium salt in view of acid-producing efficiency.

It is, therefore, desirable that the suitable anions for the anion moiety of the onium salt having a naphthalene ring at the cation moiety is an anion capable of forming a sulfonium salt.

The anion moiety of the acid generator is exemplified by fluoroalkylsulfonic acid ions or aryl sulfonic acid ions, of which hydrogen atom(s) being partially or entirely fluorinated.

The alkyl group of the fluoroalkylsulfonic acid ions may be linear, branched or cyclic and have 1 or more and 20 or less carbon atoms. Preferably, the carbon number is 1 or more and 10 or less in view of bulkiness and diffusion distance of the generated acid. In particular, branched or cyclic alkyl groups are preferable due to shorter diffusion length. Also, methyl, ethyl, propyl, butyl, octyl groups and the like are preferable due to being inexpensively synthesizable.

The aryl group of the aryl sulfonic acid ions may be an aryl group having 6 or more and 20 or less carbon atoms, and is exemplified by a phenol group or a naphthyl group that may be unsubstituted or substituted with an alkyl group or a halogen atom. In particular, aryl groups having 6 or more and 10 or less carbon atoms are preferable due to being inexpensively synthesizable. Specific examples of preferable aryl group include phenyl, toluenesulfonyl, ethylphenyl, naphthyl, methylnaphthyl groups and the like.

When hydrogen atoms in the above fluoroalkylsulfonic acid ion or the aryl sulfonic acid ion are partially or entirely substituted with a fluorine atom, the fluorination rate is preferably 10% or more and 100% or less, and more preferably 50% or more and 100% or less; it is particularly preferable that all hydrogen atoms are each substituted with a fluorine atom in view of higher acid strength. Specific examples thereof include trifluoromethane sulfonate, perfluorobutane sulfonate, perfluorooctane sulfonate, perfluorobenzene sulfonate, and the like.

Among these, the preferable anion moiety is exemplified by those represented by the following formula (a109).

[Chem. 23]

R^{120a}SO₃⁻ (a109)

In the above formula (a109), R^{120a} represents groups represented by the following formulae (a110), (a111), and (a112).

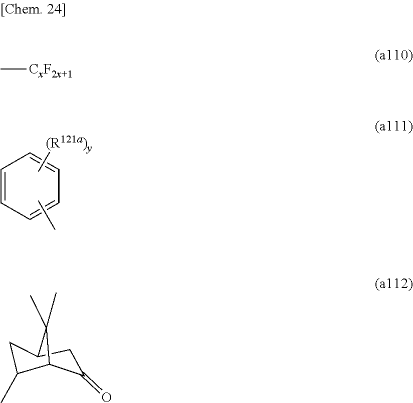

In the above formula (a110), x represents an integer of 1 or more and 4 or less. Also, in the above formula (a111), R^{121a} represents a hydrogen atom, a hydroxyl group, a linear or branched alkyl group having 1 or more and 6 or less carbon atoms, or a linear or branched alkoxy group having 1 or more and 6 or less carbon atoms, and y represents an integer of 1 or more and 3 or less. Of these, trifluoromethane sulfonate, and perfluorobutane sulfonate are preferable in view of safety.

In addition, a nitrogen-containing moiety represented by the following formulae (a113) and (a114) may also be used for the anion moiety.

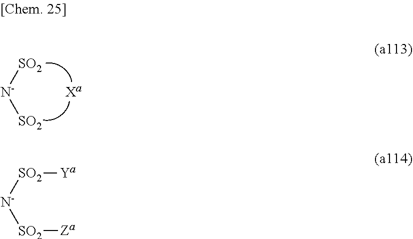

In the formulae (a113) and (a114), X^{a} represents a linear or branched alkylene group in which at least one hydrogen atom is substituted with a fluorine atom, the carbon number of the alkylene group is 2 or more and 6 or less, preferably 3 or more and 5 or less, and most preferably the carbon number is 3. In addition, Y^{a} and Z^{a} each independently represent a linear or branched alkyl group of which at least one hydrogen atom is substituted with a fluorine atom, the number of carbon atoms of the alkyl group is 1 or more and 10 or less, preferably 1 or more and 7 or less, and more preferably 1 or more and 3 or less.

The smaller number of carbon atoms in the alkylene group of X^{a}, or in the alkyl group of Y^{a} or Z^{a} is preferred since the solubility into organic solvent is favorable.

In addition, a larger number of hydrogen atoms each substituted with a fluorine atom in the alkylene group of X^{a}, or in the alkyl group of Y^{a} or Z^{a} is preferred since the acid strength becomes greater. The percentage of fluorine atoms in the alkylene group or alkyl group, i.e., the fluorination rate is preferably 70% or more and 100% or less and more preferably 90% or more and 100% or less, and most preferable are perfluoroalkylene or perfluoroalkyl groups in which all of the hydrogen atoms are each substituted with a fluorine atom.

Examples of preferable compounds for onium salts having a naphthalene ring at their cation moieties include compounds represented by the following formulae (a115) and (a116).

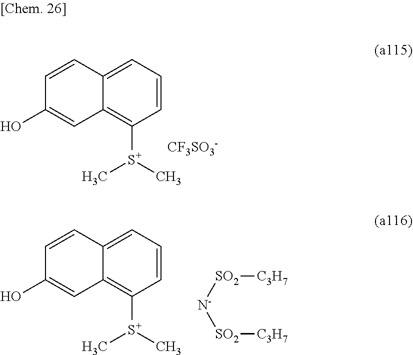

Also, the other acid generators of the fifth aspect of the acid generator (A) include bissulfonyldiazomethanes such as bis(p-toluenesulfonyl)diazomethane, bis(1,1-dimethyl ethylsulfonyl)diazomethane, bis(cyclohexylsulfonyl)diazomethane and bis(2,4-dimethylphenylsulfonyl)diazomethane; nitrobenzyl derivatives such as 2-nitrobenzyl p-toluenesulfonate, 2,6-dinitrobenzyl p-toluenesulfonate, nitrobenzyl tosylate, dinitrobenzyl tosylate, nitrobenzyl sulfonate, nitrobenzyl carbonate and dinitrobenzyl carbonate; sulfonates such as pyrogalloltrimesylate, pyrogalloltritosylate, benzyltosylate, benzylsulfonate, N-methylsulfonyloxysuccinimide, N-trichloromethylsulfonyloxysuccinimide, N-phenylsulfonyloxymaleimide and N-methylsulfonyloxyphthalimide; trifluoromethane sulfonates such as N-(trifluoromethylsulfonyloxy)phthalimide, N-(trifluoromethylsulfonyloxy)-1,8-naphthalimide and N-(trifluoromethylsulfonyloxy)-4-butyl-1,8-naphthalimide; onium salts such as diphenyliodonium hexafluorophosphate, (4-methoxyphenyl)phenyliodonium trifluoromethanesulfonate, bis(p-tert-butylphenyl)iodonium trifluoromethanesulfonate, triphenylsulfonium hexafluorophosphate, (4-methoxyphenyl) diphenylsulfonium trifluoromethanesulfonate and (p-tert-butylphenyl)diphenylsulfonium trifluoromethanesulfonate; benzointosylates such as benzointosylate and α-methylbenzointosylate; other diphenyliodonium salts, triphenylsulfonium salts, phenyldiazonium salts, benzylcarbonates and the like.

The total content of the acid generator (A) is preferably 0.01% by mass or more and 20% by mass or less, more preferably 0.03% by mass or more and 10% by mass or less, and particularly preferably 0.05% by mass or more and 8% by mass or less with respect to the total solid content of the photosensitive resin composition. Furthermore, the compound represented by the formula (A1) is preferably 0.01% by mass or more and 20% by mass or less, more preferably 0.03% by mass or more and 10% by mass or less, and particularly preferably 0.05% by mass or more and 8% by mass or less with respect to the total solid content of the photosensitive resin composition. When the use amount of the acid generator (A) is within the above range, photosensitive resin composition including a uniform solution having more excellent sensitivity, and having excellent preservation stability can be easily prepared. The compound represented by the formula (A1) is excellent in solubility in the organic solvent (S). Therefore, a photosensitive resin composition comprising the compound represented by the formula (A1) as the acid generator (A) in a desired concentration can be obtained.

### <Resin (B)>

The resin (B) whose solubility in alkali increases under action of an acid is not particularly limited, and any resin whose solubility under action of an acid increases can be used. Among them, it is preferable to contain at least one type of resin selected from the group consisting of a novolac resin (B1), a polyhydroxystyrene resin (B2), and an acrylic resin (B3).

### [Novolac Resin (B1)]

As the novolak resin (B1), a resin including a constituent unit represented by the following formula (b-11) may be used.

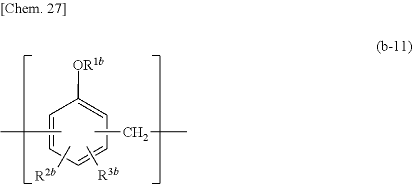

In the formula (b-11), R^{1b} represents an acid-dissociable dissolution-inhibiting group, and R^{2b} and R^{3b} each independently represent a hydrogen atom or an alkyl group having 1 or more and 6 or less carbon atoms.

The acid-dissociable dissolution-inhibiting group represented by the above R^{1b} is preferably a group represented by the following formula (b-12) or (b-13), a linear, branched or cyclic alkyl group having 1 or more and 6 or less carbon atoms, a vinyloxyethyl group, a tetrahydropyranyl group, a tetrahydrofuranyl group, or a trialkylsilyl group.

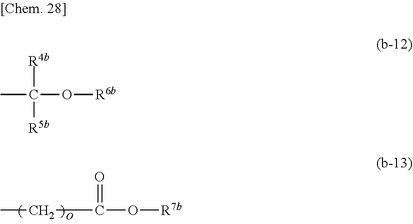

In the above formulae (b-12) and (b-13), R^{4b} and R^{5b} each independently represent a hydrogen atom, or a linear or branched alkyl group having 1 or more and 6 or less carbon atoms, R^{6b} represents a linear, branched or cyclic alkyl group having 1 or more and 10 or less carbon atoms, R^{7b} represents a linear, branched or cyclic alkyl group having 1 or more and 6 or less carbon atoms, and o represents 0 or 1.

Examples of the above linear or branched alkyl group include a methyl group, an ethyl group, a propyl group, an isopropyl group, an n-butyl group, an isobutyl group, a tert-butyl group, a pentyl group, an isopentyl group, a neopentyl group, and the like. Also, examples of the above cyclic alkyl group include a cyclopentyl group, a cyclohexyl group, and the like.

Specific examples of the acid-dissociable dissolution-inhibiting group represented by the above formula (b-12) include a methoxyethyl group, ethoxyethyl group, n-propoxyethyl group, isopropoxyethyl group, n-butoxyethyl group, isobutoxyethyl group, tert-butoxyethyl group, cyclohexyloxyethyl group, methoxypropyl group, ethoxypropyl group, 1-methoxy-1-methyl-ethyl group, 1-ethoxy-1-methylethyl group, and the like. Furthermore, specific examples of the acid-dissociable dissolution-inhibiting group represented by the above formula (b-13) include a tert-butoxycarbonyl group, a tert-butoxycarbonylmethyl group, and the like. Examples of the above trialkylsilyl group include a trimethylsilyl group and tri-tert-butyldimethylsilyl group in which each alkyl group has 1 or more and 6 or less carbon atoms.

### [Polyhydroxystyrene Resin (B2)]

As the polyhydroxystyrene resin (B2), a resin including a constituent unit represented by the following formula (b4) may be used.

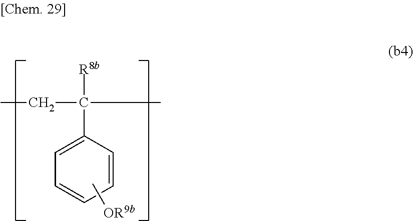

In the above formula (b4), R^{8b} represents a hydrogen atom or an alkyl group having 1 or more and 6 or less carbon atoms, and R^{9b} represents an acid-dissociable dissolution-inhibiting group.

The above alkyl group having 1 or more and 6 or less carbon atoms may include, for example, linear, branched or cyclic alkyl groups having 1 or more and 6 or less carbon atoms. Examples of the linear or branched alkyl group include a methyl group, ethyl group, propyl group, isopropyl group, n-butyl group, isobutyl group, tert-butyl group, pentyl group, isopentyl group, neopentyl group, and the like. Examples of the cyclic alkyl group include a cyclopentyl group and cyclohexyl group.

The acid-dissociable dissolution-inhibiting group represented by the above R^{9b} may be similar to the acid-dissociable dissolution-inhibiting groups exemplified in terms of the above formulae (b-12) and (b-13).

Furthermore, the polyhydroxystyrene resin (B2) may include another polymerizable compound as a constituent unit in order to moderately control physical or chemical properties. The polymerizable compound is exemplified by conventional radical polymerizable compounds and anion polymerizable compounds. Examples of the polymerizable compound include monocarboxylic acids such as acrylic acid, methacrylic acid and crotonic acid; dicarboxylic acids such as maleic acid, fumaric acid and itaconic acid; methacrylic acid derivatives having a carboxyl group and an ester bond such as 2-methacryloyloxyethyl succinic acid, 2-methacryloyloxyethyl maleic acid, 2-methacryloyloxyethyl phthalic acid and 2-methacryloyloxyethyl hexahydrophthalic acid; (meth)acrylic acid alkyl esters such as methyl (meth)acrylate, ethyl (meth)acrylate and butyl (meth)acrylate; (meth)acrylic acid hydroxyalkyl esters such as 2-hydroxyethyl (meth)acrylate and 2-hydroxypropyl (meth)acrylate; (meth)acrylic acid aryl esters such as phenyl (meth)acrylate and benzyl (meth)acrylate; dicarboxylic acid diesters such as diethyl maleate and dibutyl fumarate; vinyl group-containing aromatic compounds such as styrene, α-methylstyrene, chlorostyrene, chloromethylstyrene, vinyltoluene, hydroxystyrene, α-methylhydroxystyrene and α-ethylhydroxystyrene; vinyl group-containing aliphatic compounds such as vinyl acetate; conjugated diolefins such as butadiene and isoprene; nitrile group-containing polymerizable compounds such as acrylonitrile and methacrylonitrile; chlorine-containing polymerizable compounds such as vinyl chloride and vinylidene chloride; and amide bond-containing polymerizable compounds such as acrylamide and methacrylamide.

### [Acrylic Resin (B3)]

The acrylic resin (B3) is not particularly limited as long as it is an acrylic resin the solubility of which in alkali increases under the action of acid, and has conventionally blended in various photosensitive resin compositions. Preferably, the acrylic resin (B3) contains a constituent unit (b-3) derived from, for example, an acrylic ester including an —SO₂-containing cyclic group or a lactone-containing cyclic group. In such a case, when a resist pattern is formed, a resist pattern having a preferable cross-sectional shape can be easily formed.

### (—SO₂—Containing Cyclic Group)

Herein, the “—SO₂-containing cyclic group” refers to a cyclic group having a cyclic group containing a ring including —SO₂— in the ring skeleton thereof, specifically a cyclic group in which the sulfur atom (S) in —SO₂— forms a part of the ring skeleton of the cyclic group. Considering a ring including —SO₂— in the ring skeleton thereof as the first ring, a group having that ring alone is called a monocyclic group, and a group further having another ring structure is called a polycyclic group regardless of its structure. The —SO₂-containing cyclic group may be monocyclic or polycyclic.

In particular, the —SO₂-containing cyclic group is preferably a cyclic group containing —O—SO₂— in the ring skeleton thereof, i.e., a cyclic group containing a sultone ring in which —O—S— in —O—SO₂— forms a part of the ring skeleton.

The number of carbon atoms in an —SO₂-containing cyclic group is preferably 3 or more and 30 or less, more preferably 4 or more and 20 or less, even more preferably 4 or more and 15 or less, and in particular preferably 4 or more and 12 or less. The above number of carbon atoms is the number of carbon atoms constituting a ring skeleton, and shall not include the number of carbon atoms in a substituent.

The —SO₂-containing cyclic group may be an —SO₂-containing aliphatic cyclic group or an —SO₂-containing aromatic cyclic group. It is preferably an —SO₂-containing aliphatic cyclic group.

—SO₂-containing aliphatic cyclic groups include a group in which at least one hydrogen atom is removed from an aliphatic hydrocarbon ring where a part of the carbon atoms constituting the ring skeleton thereof is(are) substituted with —SO₂— or —O—SO₂—. More specifically, they include a group in which at least one hydrogen atom is removed from an aliphatic hydrocarbon ring where —CH₂— constituting the ring skeleton thereof is substituted with —SO₂— and a group in which at least one hydrogen atom is removed from an aliphatic hydrocarbon ring where —CH₂—CH₂— constituting the ring thereof is substituted with —O—SO₂—.

The number of carbon atoms in the above alicyclic hydrocarbon ring is preferably 3 or more and 20 or less, more preferably 3 or more and 12 or less. The above alicyclic hydrocarbon ring may be polycyclic, or may be monocyclic. As the monocyclic alicyclic hydrocarbon group, preferred is a group in which two hydrogen atoms are removed from monocycloalkane having 3 or more and 6 or less carbon atoms. Examples of the above monocycloalkane can include cyclopentane, cyclohexane and the like. As the polycyclic alicyclic hydrocarbon ring, preferred is a group in which two hydrogen atoms are removed from polycycloalkane having 7 or more and 12 or less carbon atoms, and specific examples of the above polycycloalkane include adamantane, norbornane, isobornane, tricyclodecane, tetracyclododecane and the like.

The —SO₂-containing cyclic group may have a substituent. Examples of the above substituent include, for example, an alkyl group, an alkoxy group, a halogen atom, a halogenated alkyl group, a hydroxy group, an oxygen atom (═O), —COOR″, —OC(═O)R″, a hydroxyalkyl group, a cyano group and the like.

For an alkyl group as the above substituent, preferred is an alkyl group having 1 or more and 6 or less carbon atoms. The above alkyl group is preferably linear or branched. Specific examples include a methyl group, an ethyl group, an n-propyl group, an isopropyl group, an n-butyl group, an isobutyl group, a tert-butyl group, an n-pentyl group, an isopentyl group, a neopentyl group, an n-hexyl group and the like. Among these, a methyl group or an ethyl group is preferred, and a methyl group is particularly preferred.

For an alkoxy group as the above substituent, preferred is an alkoxy group having 1 or more and 6 or less carbon atoms. The above alkoxy group is preferably linear or branched. Specific examples include a group in which an alkyl groups recited as an alkyl group for the above substituent is attached to the oxygen atom (—O—).

Halogen atoms as the above substituent include a fluorine atom, a chlorine atom, a bromine atom, an iodine atom and the like, and a fluorine atom is preferred.

Halogenated alkyl groups for the above substituent include a group in which a part or all of the hydrogen atoms in the above alkyl group is(are) substituted with the above halogen atom(s).

Halogenated alkyl groups as the above substituent include a group in which a part or all of the hydrogen atoms in the alkyl groups recited as an alkyl group for the above substituent is(are) substituted with the above halogen atom(s). As the above halogenated alkyl group, a fluorinated alkyl group is preferred, and a perfluoroalkyl group is particularly preferred.

R″s in the aforementioned —COOR″ and —OC(═O)R″ are either a hydrogen atom or a linear, branched or cyclic alkyl group having 1 or more and 15 or less carbon atoms.

In a case where R″ is a linear or branched alkyl group, the number of carbon atoms in the above chain alkyl group is preferably 1 or more and 10 or less, more preferably 1 or more and 5 or less, and in particular preferably 1 or 2.

In a case where R″ is a cyclic alkyl group, the number of carbon atoms in the above cyclic alkyl group is preferably 3 or more and 15 or less, more preferably 4 or more and 12 or less, and in particular preferably 5 or more and 10 or less. Specific examples can include a group in which one or more hydrogen atoms are removed from monocycloalkane, and polycycloalkane such as bicycloalkane, tricycloalkane, tetracycloalkane, and the like, which may be or not may be substituted with a fluorine atom or a fluorinated alkyl group. More specific examples include a group in which one or more hydrogen atoms are removed from monocycloalkane such as cyclopentane and cyclohexane; and polycycloalkane such as adamantane, norbornane, isobornane, tricyclodecane and tetracyclododecane.

For a hydroxyalkyl group as the above substituent, preferred is a hydroxyalkyl group having 1 or more and 6 or less carbon atoms. Specific examples include a group in which at least one of the hydrogen atoms in the alkyl groups recited as an alkyl group for the above substituent is substituted with a hydroxy group.

More specific examples of the —SO₂-containing cyclic group include the groups represented by the following formulae (3-1) to (3-4).

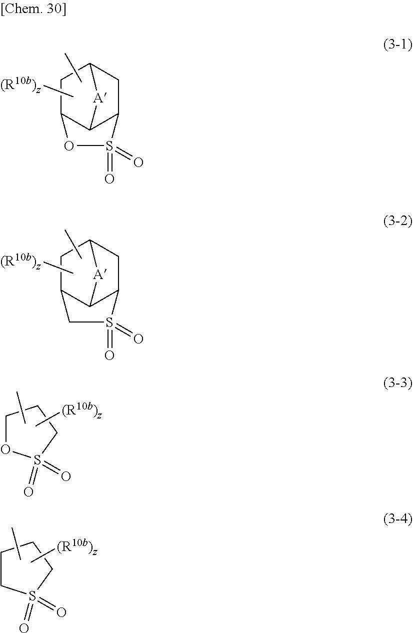

(In the formulae, A′ represents an alkylene group having 1 or more and 5 or less carbon atoms optionally including an oxygen atom or a sulfur atom, an oxygen atom or a sulfur atom; z represents an integer of 0 or more and 2 or less; R^{10b} represents an alkyl group, an alkoxy group, a halogenated alkyl group, a hydroxy group, —COOR″, —OC(═O)R″, a hydroxyalkyl group, or a cyano group; and R″ represents a hydrogen atom or an alkyl group.)

In the above formulae (3-1) to (3-4), A′ represents an alkylene group having 1 or more and 5 or less carbon atoms optionally including an oxygen atom (—O—) or a sulfur atom (—S—), an oxygen atom or a sulfur atom. As an alkylene group having 1 or more and 5 or less carbon atoms in A′, a linear or branched alkylene group is preferred, and examples thereof include a methylene group, an ethylene group, an n-propylene group, an isopropylene group and the like.

In a case where the above alkylene group includes an oxygen atom or a sulfur atom, specific examples thereof include a group in which —O— or —S— is present at a terminal or between carbon atoms of the above alkylene group, for example, —O—CH₂—, —CH₂—O—CH₂—, —S—CH₂—, —CH₂—S—CH₂—, and the like. As A′, an alkylene group having 1 or more and 5 or less carbon atoms or —O— is preferred, and an alkylene group having 1 or more and 5 or less carbon atoms is more preferred, and a methylene group is most preferred.

z may be any of 0, 1, and 2, and is most preferably 0. In a case where z is 2, a plurality of R^{10b} may be the same, or may differ from each other.

An alkyl group, an alkoxy group, a halogenated alkyl group, —COOR″, —OC(═O)R″ and a hydroxyalkyl group in R^{10b} include those similar to the groups described above for the alkyl group, the alkoxy group, the halogenated alkyl group, —COOR″, —OC(═O)R″ and the hydroxyalkyl group, respectively, which are recited as those optionally contained in the —SO₂-containing cyclic group.

Below, specific cyclic groups represented by the above formulae (3-1) to (3-4) will be illustrated. Note here that “Ac” in the formulae represents an acetyl group.

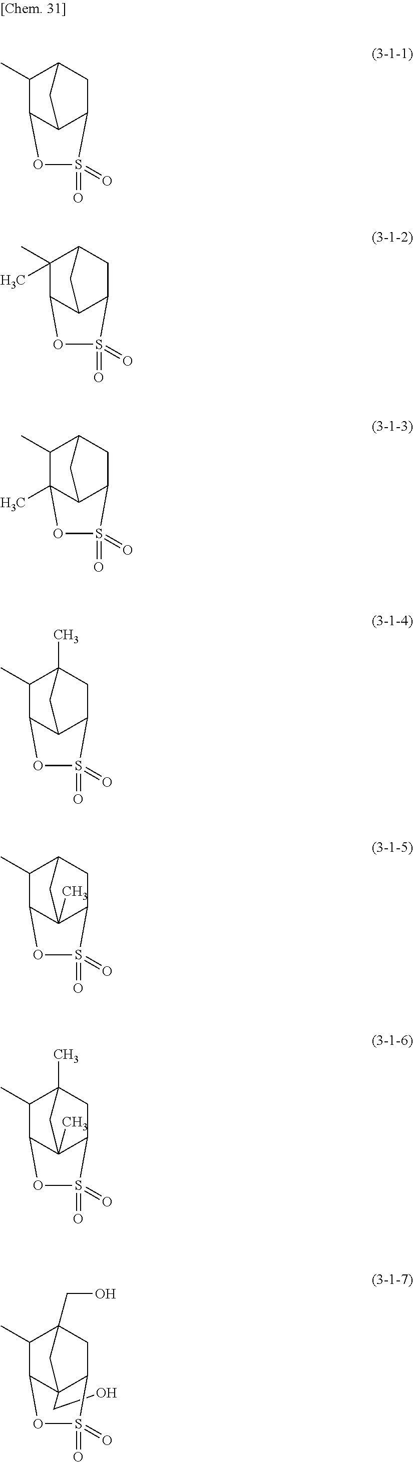

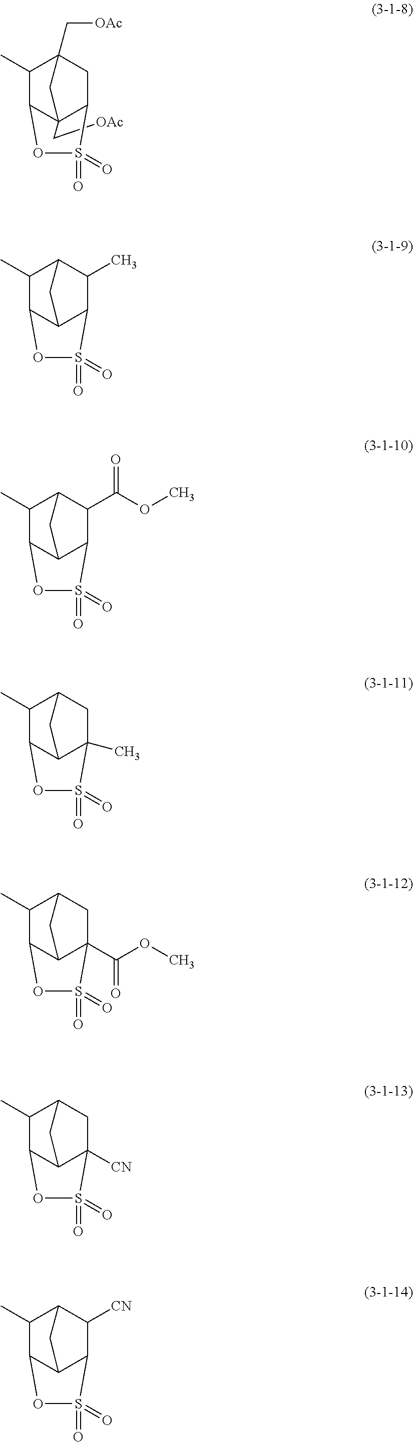

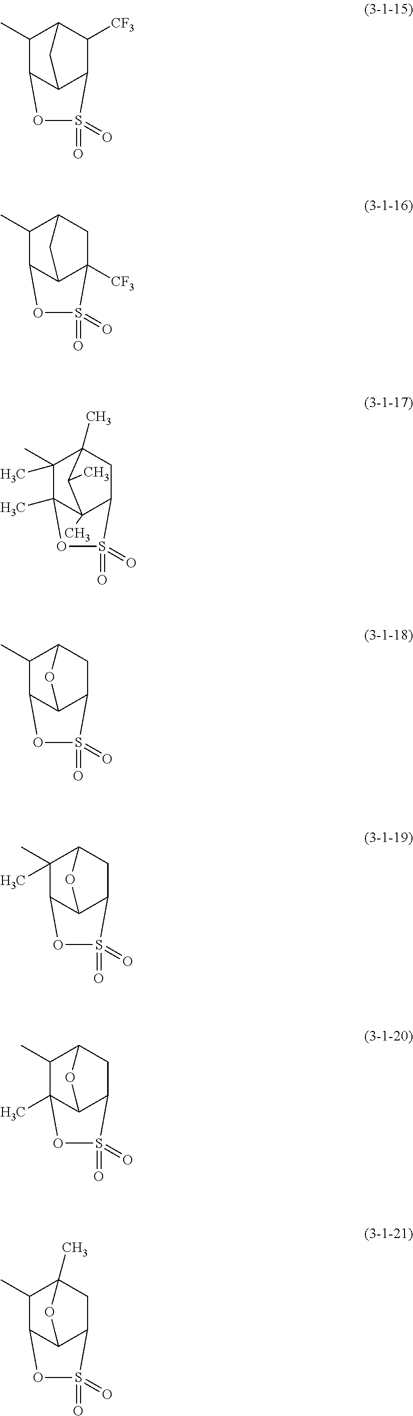

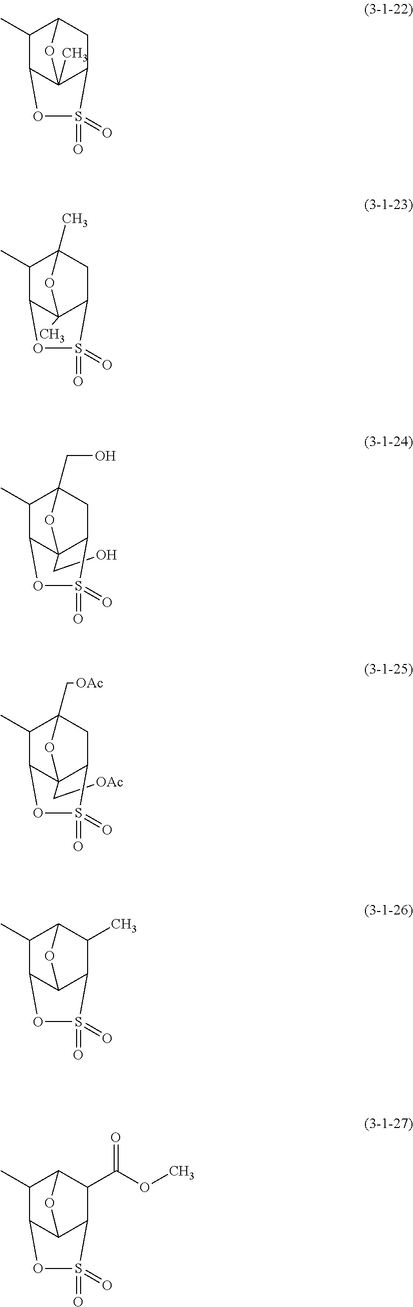

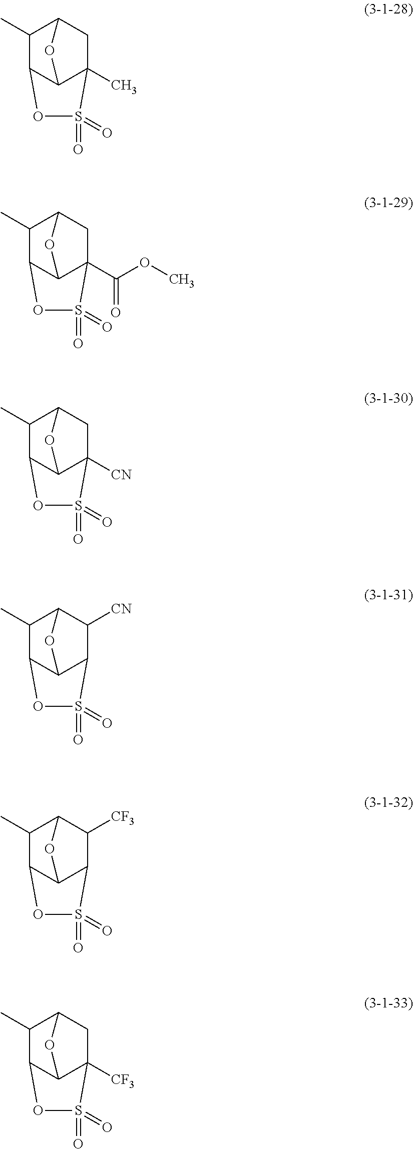

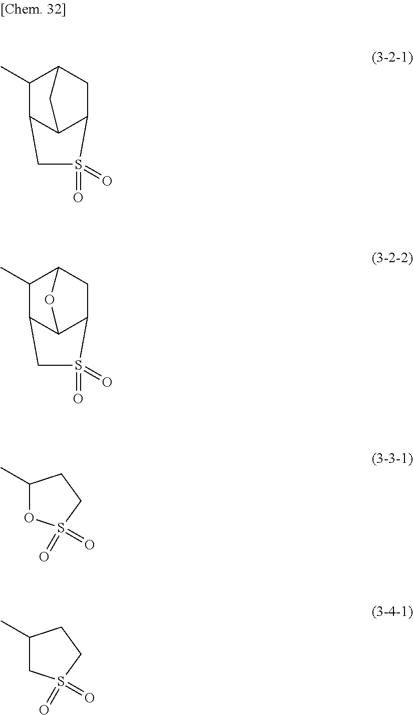

As the —SO₂-containing cyclic group, among those shown above, a group represented by the above formula (3-1) is preferred, and at least one selected from the group consisting of the groups represented by any of the aforementioned formulae (3-1-1), (3-1-18), (3-3-1) and (3-4-1) is more preferred, and a group represented by the aforementioned formula (3-1-1) is most preferred.

### (Lactone-Containing Cyclic Group)

The “lactone-containing cyclic group” refers to a cyclic group containing a ring (lactone ring) including —O—C(═O)— in the ring skeleton thereof. Considering the lactone ring as the first ring, a group having that lactone ring alone is called a monocyclic group, and a group further having another ring structure is called a polycyclic group regardless of its structure. The lactone-containing cyclic group may be a monocyclic group, or may be a polycyclic group.

There is no particular limitation on the lactone cyclic group in the constituent unit (b-3), and any cyclic group containing lactone can be used. Specifically, examples of the lactone-containing monocyclic groups include a group in which one hydrogen atom is removed from 4 to 6 membered ring lactone, for example, a group in which one hydrogen atom is removed from β-propionolactone, a group in which one hydrogen atom is removed from γ-butyrolactone, a group in which one hydrogen atom is removed from δ-valerolactone and the like. Further, lactone-containing polycyclic groups include a group in which one hydrogen atom is removed from bicycloalkane, tricycloalkane and tetracycloalkane having a lactone ring.

As to the constituent unit (b-3), as long as the constituent unit (b-3) has an —SO₂-containing cyclic group or a lactone-containing cyclic group, the structures of other parts are not particularly limited. A preferred constituent unit (b-3) is at least one constituent unit selected from the group consisting of a constituent unit (b-3-S) derived from an acrylic acid ester including an —SO₂-containing cyclic group in which a hydrogen atom attached to the carbon atom in the a position may be substituted with a substituent; and a constituent unit (b-3-L) derived from an acrylic acid ester including a lactone-containing cyclic group in which the hydrogen atom attached to the carbon atom in the a position may be substituted with a substituent.

[Constituent Unit (b-3-S)]

More specifically, examples of the constituent unit (b-3-S) include one represented by the following formula (b-S1).

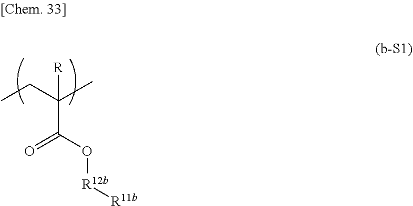

(In the formula, R represents a hydrogen atom, an alkyl group having 1 or more 5 or less carbon atoms or a halogenated alkyl group having 1 or more 5 or less carbon atoms; and R^{11b} represents an —SO₂-containing cyclic group; and R^{12b} represents a single-bond or divalent linking group.)

In the formula (b-S1), R is similarly defined as above. R^{12b} is similarly defined as in the —SO₂-containing cyclic group described above. R^{12b} may be either a single-bond or a divalent linking group. R^{12b} is preferably a divalent linking group, since the effect of the present invention is excellent when R^{12b} is a divalent linking group.

There is no particular limitation on the divalent linking group in R^{12b}, and suitable examples include an optionally substituted divalent hydrocarbon group, a divalent linking group including a heteroatom, and the like.

Optionally Substituted Divalent Hydrocarbon Group

The hydrocarbon group as a divalent linking group may be an aliphatic hydrocarbon group, or may be an aromatic hydrocarbon group. The aliphatic hydrocarbon group means a hydrocarbon group without aromaticity. The above aliphatic hydrocarbon group may be saturated or may be unsaturated. Usually, a saturated hydrocarbon group is preferred. More specifically, examples of the above aliphatic hydrocarbon group include a linear or branched aliphatic hydrocarbon group, an aliphatic hydrocarbon group including a ring in the structure thereof and the like.

The number of carbon atoms in the linear or branched aliphatic hydrocarbon group is preferably 1 or more and 10 or less, more preferably 1 or more and 8 or less, and even more preferably 1 or more and 5 or less.

As the linear aliphatic hydrocarbon group, a linear alkylene group is preferred. Specific examples include a methylene group [—CH₂—], an ethylene group [—(CH₂)₂—], a trimethylene group [—(CH₂)₃—], a tetramethylene group [—(CH₂)₄—], a pentamethylene group [—(CH₂)₅—] and the like.

As the branched aliphatic hydrocarbon group, a branched alkylene group is preferred. Specific examples include alkyl alkylene groups such as alkyl methylene groups such as —CH(CH₃)—, —CH(CH₂CH₃)—, —C(CH₃)₂—, —C(CH₃)(CH₂CH₃)—, —C(CH₃)(CH₂CH₂CH₃)— and —C(CH₂CH₃)₂—; alkyl ethylene groups such as —CH(CH₃)CH₂—, —CH(CH₃)CH(CH₃)—, —C(CH₃)₂CH₂—, —CH(CH₂CH₃)CH₂— and —C(CH₂CH₃)₂—CH₂—; alkyl trimethylene groups such as —CH(CH₃)CH₂CH₂— and —CH₂CH(CH₃)CH₂—; alkyl tetramethylene groups such as —CH(CH₃)CH₂CH₂CH₂— and —CH₂CH(CH₃)CH₂CH₂—; and the like. As an alkyl group in the alkyl alkylene group, a linear alkyl group having 1 or more and 5 or less carbon atoms is preferred.

The above linear or branched aliphatic hydrocarbon group may or may not have a substituent (a group or atom other than a hydrogen atom) which substitutes a hydrogen atom. Examples of the substituent include a fluorine atom, a fluorinated alkyl group having 1 or more and 5 or less carbon atoms substituted with a fluorine atom, an oxo group (═O) and the like.

Examples of the above aliphatic hydrocarbon group including a ring in the structure thereof include a cyclic aliphatic hydrocarbon group optionally including a hetero atom in the ring structure (a group in which two hydrogen atoms are removed from an aliphatic hydrocarbon ring); a group in which the above cyclic aliphatic hydrocarbon group is attached to an end of a linear or branched aliphatic hydrocarbon group; a group in which the above cyclic aliphatic hydrocarbon group is present in a linear or branched aliphatic hydrocarbon group along the chain; and the like. Examples of the above linear or branched aliphatic hydrocarbon group include groups similar to the above.

The number of carbon atoms in the cyclic aliphatic hydrocarbon group is preferably 3 or more and 20 or less, and more preferably 3 or more and 12 or less.

The cyclic aliphatic hydrocarbon group may be polycyclic, or may be monocyclic. As the monocyclic aliphatic hydrocarbon group, a group in which two hydrogen atoms are removed from monocycloalkane is preferred. The number of carbon atoms in the above monocycloalkane is preferably 3 or more and 6 or less. Specific examples include cyclopentane, cyclohexane and the like. As the polycyclic aliphatic hydrocarbon group, a group in which two hydrogen atoms are removed from polycycloalkane is preferred. The number of carbon atoms in the above polycycloalkane is preferably 7 or more and 12 or less. Specific examples include adamantane, norbornane, isobornane, tricyclodecane, tetracyclododecane and the like.

The cyclic aliphatic hydrocarbon group may or may not have a substituent which substitutes a hydrogen atom (a group or atom other than a hydrogen atom). Examples of the above substituent include an alkyl group, an alkoxy group, a halogen atom, a halogenated alkyl group, a hydroxy group, an oxo group (═O) and the like.

For an alkyl group as the above substituent, an alkyl group having 1 or more and 5 or less carbon atoms is preferred, and a methyl group, an ethyl group, a propyl group, an n-butyl group and a tert-butyl group are more preferred.

For an alkoxy group as the above substituent, an alkoxy group having 1 or more and 5 or less carbon atoms is preferred, and a methoxy group, an ethoxy group, an n-propoxy group, an iso-propoxy group, an n-butoxy group and a tert-butoxy group are more preferred, and a methoxy group and an ethoxy group are particularly preferred.

Halogen atoms as the above substituent include a fluorine atom, a chlorine atom, a bromine atom, an iodine atom and the like, and a fluorine atom is preferred.

Halogenated alkyl groups as the above substituent include a group in which a part or all of hydrogen atoms in the aforementioned alkyl group is(are) substituted with the above halogen atom(s).

In the cyclic aliphatic hydrocarbon group, a part of carbon atoms constituting the ring structure thereof may be substituted with —O—, or —S—. As the substituent including the above hetero atom, preferred are —O—, —C(═O)—O—, —S—, —S(═O)₂— and —S(═O)₂—O—.

The aromatic hydrocarbon group as the divalent hydrocarbon group is a divalent hydrocarbon group having at least one aromatic ring, and may have a substituent. There is no particular limitation on the aromatic ring as long as it is a cyclic conjugated system having a 4n+2π electrons, and it may be monocyclic or may be polycyclic. The number of carbon atoms in the aromatic ring is preferably 5 or more and 30 or less, more preferably 5 or more and 20 or less, further more preferably 6 or more and 15 or less, and particularly preferably 6 or more and 12 or less. However, the number of carbon atoms in a substituent shall not be included in the above number of carbon atoms.

Specifically, aromatic rings include aromatic hydrocarbon rings such as benzene, naphthalene, anthracene and phenanthrene; aromatic heterocycles in which a part of the carbon atoms constituting the above aromatic hydrocarbon ring is(are) substituted with hetero atom(s). Hetero atoms in the aromatic heterocycle include an oxygen atom, a sulfur atom, a nitrogen atom and the like. Specifically, aromatic heterocycles include a pyridine ring, a thiophene ring, and the like.

Specific examples of the aromatic hydrocarbon group as a divalent hydrocarbon group include a group in which two hydrogen atoms are removed from the above aromatic hydrocarbon ring or the above aromatic heterocycle (an arylene group or a heteroarylene group); a group in which two hydrogen atoms are removed from an aromatic compound including two or more aromatic rings (for example, biphenyl, fluorene and the like); a group in which one hydrogen atom from a group where one hydrogen atom is removed from the above aromatic hydrocarbon ring or the above aromatic heterocycle (an aryl group or a heteroaryl group) is substituted with an alkylene group (for example, a group in which one hydrogen atom is further removed from an aryl group in an arylalkyl group such as a benzyl group, a phenethyl group, a 1-naphthylmethyl group, a 2-naphthylmethyl group, a 1-naphthylethyl group and a 2-naphthylethyl group); and the like.

The number of carbon atoms in the above alkylene group bonded to an aryl group or a heteroaryl group is preferably 1 or more and 4 or less, more preferably 1 or more and 2 or less, and particularly preferably 1.

In the above aromatic hydrocarbon group, a hydrogen atom of the above aromatic hydrocarbon group may be substituted with a substituent. For example, a hydrogen atom attached to an aromatic ring in the above aromatic hydrocarbon group may be substituted with a substituent. Examples of the substituent include an alkyl group, an alkoxy group, a halogen atom, a halogenated alkyl group, a hydroxy group, an oxo group (═O) and the like.

For an alkyl group as the above substituent, an alkyl group having 1 or more and 5 or less carbon atoms is preferred, and a methyl group, an ethyl group, an n-propyl group, an n-butyl group and a tert-butyl group are more preferred.

For an alkoxy group as the above substituent, an alkoxy group having 1 or more and 5 or less carbon atoms is preferred, and a methoxy group, an ethoxy group, an n-propoxy group, an iso-propoxy group, an n-butoxy group and a tert-butoxy group are preferred, and a methoxy group and an ethoxy group are more preferred.

Halogen atoms as the above substituent include a fluorine atom, a chlorine atom, a bromine atom, an iodine atom and the like, and a fluorine atom is preferred.

Halogenated alkyl groups as the above substituent include a group in which a part or all of hydrogen atoms in the aforementioned alkyl group is(are) substituted with the above halogen atom(s).

Divalent Linking Group Including Hetero Atom

A hetero atom in the divalent linking group including a hetero atom is an atom other than a carbon atom and a hydrogen atom, and examples thereof include an oxygen atom, a nitrogen atom, a sulfur atom, a halogen atom and the like.

Specific examples of the divalent linking group including a hetero atom include non-hydrocarbon based linking groups such as —O—, —C(═O)—, —C(═O)—O—, —O—C(═O)—O—, —S—, —S(═O)₂—, —S(═O)₂—O—, —NH—, —NH—C(═O)—, —NH—C(═NH)—, ═N—, and combinations of at least one of these non-hydrocarbon based linking groups and a divalent hydrocarbon group and the like. Examples of the above divalent hydrocarbon group include those similar to the aforementioned divalent hydrocarbon groups optionally having a substituent, and linear or branched aliphatic hydrocarbon groups are preferred.

Among those described above, H in —NH— in —C(═O)—NH—, —NH— and —NH—C(═NH)— may be substituted with a substituent such as an alkyl group or an acyl group, respectively. The number of carbon atoms in the above substituent is preferably 1 or more and 10 or less, more preferably 1 or more and 8 or less, and in particular preferably 1 or more and 5 or less.

As a divalent linking group in R^{12b}, a linear or branched alkylene group, a cyclic aliphatic hydrocarbon group, or a divalent linking group including a hetero atom is preferred.

In a case where the divalent linking group in R^{12b} is a linear or branched alkylene group, the number of carbon atoms in the above alkylene group is preferably 1 or more and 10 or less, more preferably 1 or more and 6 or less, in particular preferably 1 or more and 4 or less, and most preferably 1 or more and 3 or less. Specific examples include groups similar to the linear alkylene groups or branched alkylene groups recited as a linear and branched aliphatic hydrocarbon group in the description of the “divalent hydrocarbon group optionally having a substituent” as the aforementioned divalent linking group.

In a case where the divalent linking group in R^{12b} is a cyclic aliphatic hydrocarbon group, examples of the above cyclic aliphatic hydrocarbon group include groups similar to the cyclic aliphatic hydrocarbon groups recited as the “aliphatic hydrocarbon group including a ring in the structure” in the description of the “divalent hydrocarbon group optionally having a substituent” as the aforementioned divalent linking group.

As the above cyclic aliphatic hydrocarbon group, particularly preferred is a group in which two or more hydrogen atoms are removed from cyclopentane, cyclohexane, norbornane, isobornane, adamantane, tricyclodecane or tetracyclododecane.

In a case where the divalent linking group in R^{12b} is a divalent linking group including a hetero atom, groups preferred as the above linking groups include —O—, —C(═O)—O—, —C(═O)—, —O—C(═O)—O—, —C(═O)—NH—, —NH— (H may be substituted with a substituent such as an alkyl group or an acyl group), —S—, —S(═O)₂—, —S(═O)₂—O— and a group represented by the general formula —Y^{1b}—O—Y^{2b}—, —[Y^{1b}—C(═O)—O]_{m′}—Y^{2b} or (═O)—Y^{2b}— [wherein in the formula, Y^{1b} and Y^{2b} are divalent hydrocarbon groups each independently, optionally having a substituent, and O represents an oxygen atom, and m′ is an integer of 0 or more and 3 or less], or the like.

In a case where the divalent linking group in R^{12b} is —NH—, the hydrogen atom in —NH— may be substituted with a substituent such as an alkyl group or an acyl group. The number of carbon atoms in the above substituent (an alkyl group, an acyl group and the like) is preferably 1 or more and 10 or less, more preferably 1 or more and 8 or less, and in particular preferably 1 or more and 5 or less.

Y^{1b} and Y^{2b} in the formula —Y^{1b}—O—Y^{2b}—, —[Y^{1b}—C(═O)—O]_{m′}—Y^{2b}— or —Y^{1b}—O—C(═O)—Y^{2b}— are divalent hydrocarbon groups each independently, optionally having a substituent. Examples of the above divalent hydrocarbon group include groups similar to the “divalent hydrocarbon group optionally having a substituent” recited in the description of the above divalent linking group.

As Y^{1b}, a linear aliphatic hydrocarbon group is preferred, and a linear alkylene group is more preferred, and a linear alkylene group having 1 or more and 5 or less carbon atoms is more preferred, and a methylene group and an ethylene group are particularly preferred.

As Y^{2b}, a linear or branched aliphatic hydrocarbon group is preferred, and a methylene group, an ethylene group and an alkylmethylene group are more preferred. The alkyl group in the above alkylmethylene group is preferably a linear alkyl group having 1 or more and 5 or less carbon atoms, more preferably a linear alkyl group having 1 or more and 3 or less carbon atoms, and particularly preferably a methyl group.

In a group represented by the formula —[Y^{1b}—C(═O)—O]_{m′}—Y^{2b}—, m′ is an integer of 0 or more and 3 or less, preferably an integer of 0 or more and 2 or less, more preferably 0 or 1, and particularly preferably 1. In other words, as a group represented by the formula —[Y^{1b}—C(═O)—O]_{m′}—Y^{2b}—, a group represented by the formula —Y^{1b}—C(═O)—O—Y^{2b}— is particularly preferred. Among these, a group represented by the formula —(CH₂)_{a′}—C(═O)—O—(CH₂)_{b′}— is preferred. In the above formula, a′ is an integer of 1 or more and 10 or less, preferably an integer of 1 or more and 8 or less, more preferably an integer of 1 or more and 5 or less, even more preferably 1 or 2, and most preferably 1. b′ is an integer of 1 or more and 10 or less, preferably an integer of 1 or more and 8 or less, more preferably an integer of 1 or more and 5 or less, even more preferably 1 or 2, and most preferably 1.

With regard to the divalent linking group in R^{12b}, an organic group including a combination of at least one non-hydrocarbon group and a divalent hydrocarbon group is preferred as the divalent linking group including a hetero atom. Among these, a linear chain group having an oxygen atom as a hetero atom, for example, a group including an ether bond or an ester bond is preferred, and a group represented by the aforementioned formula —Y^{1b}—O—Y^{2b}—, —[Y^{1b}—C(═O)—O]_{m′}—Y^{2b}— or —Y^{1b}—O—C(═O)—Y^{2b}— is more preferred, and a group represented by the aforementioned formula —[Y^{1b}—C(═O)—O]_{m′}—Y^{2b}— or —Y^{1b}—O—C(═O)—Y^{2b}— is particularly preferred.

As the divalent linking group in R^{12b}, a group including an alkylene group or an ester bond (—C(═O)—O—) is preferred.

The above alkylene group is preferably a linear or branched alkylene group. Suitable examples of the above linear aliphatic hydrocarbon group include a methylene group [—CH₂—], an ethylene group [—(CH₂)₂—], a trimethylene group [—(CH₂)₃—], a tetramethylene group [—(CH₂)₄—], a pentamethylene group [—(CH₂)₅—] and the like. Suitable examples of the above branched alkylene group include alkyl alkylene groups such as alkyl methylene groups such as —CH(CH₃)—, —CH(CH₂CH₃)—, —C(CH₃)₂—, —C(CH₃)(CH₂CH₃)—, —C(CH₃)(CH₂CH₂CH₃)— and —C(CH₂CH₃)₂—; alkyl ethylene groups such as —CH(CH₃)CH₂—, —CH(CH₃)CH(CH₃)—, —C(CH₃)₂CH₂—, —CH(CH₂CH₃)CH₂— and —C(CH₂CH₃)₂—CH₂—; alkyl trimethylene groups such as —CH(CH₃)CH₂CH₂— and —CH₂CH(CH₃)CH₂—; alkyl tetramethylene groups such as —CH(CH₃)CH₂CH₂CH₂— and —CH₂CH(CH₃)CH₂CH₂—.

As the divalent linking group including an ester bond, particularly preferred is a group represented by the formula: —R^{13b}—C(═O)—O—[wherein R^{13b} represents a divalent linking group.]. In other words, the constituent unit (b-3-S) is preferably a constituent unit represented by the following formula (b-S1-1).

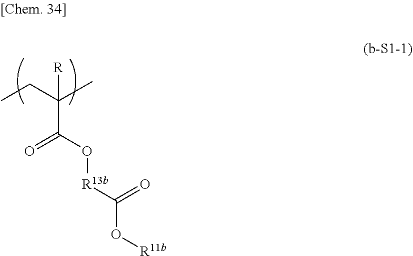

(In the formula, R and R^{11b} are each similar to the above, and R^{13b} represents a divalent linking group.)

There is no particular limitation for R^{13b}, examples thereof include those similar to the aforementioned divalent linking group in R^{12b}. As the divalent linking group in R^{13b}, a linear or branched alkylene group, an aliphatic hydrocarbon group including a ring in the structure, or a divalent linking group including a hetero atom is preferred, and a linear or branched alkylene group or a divalent linking group including an oxygen atom as a hetero atom is preferred.

As the linear alkylene group, a methylene group or an ethylene group is preferred, and a methylene group is particularly preferred. As the branched alkylene group, an alkylmethylene group or an alkylethylene group is preferred, and —CH(CH₃)—, —C(CH₃)₂— or —C(CH₃)₂CH₂— is particularly preferred.

As the divalent linking group including an oxygen atom, a divalent linking group including an ether bond or an ester bond is preferred, and the aforementioned —Y^{1b}—O—Y^{2b}, —[Y^{1b}—C(═O)—O]_{m′}—Y^{2b}— or —Y^{1b}—O—C(═O)—Y^{2b}— is more preferred. Y^{1b} and Y^{2b} are each independently divalent hydrocarbon groups optionally having a substituent, and m′ is an integer of 0 or more and 3 or less. Among these, —Y^{1b}—O—C(═O)—Y^{2b}— is preferred, and a group represented by —(CH₂)_{c}—O—C(═O)—(CH₂)_{d}— is particularly preferred. c is an integer of 1 or more and 5 or less, and 1 or 2 is preferred. d is an integer of 1 or more and 5 or less, and 1 or 2 is preferred.

As the constituent unit (b-3-S), in particular, one represented by the following formula (b-S1-11) or (b-S1-12) is preferred, and one represented by the formula (b-S1-12) is more preferred.

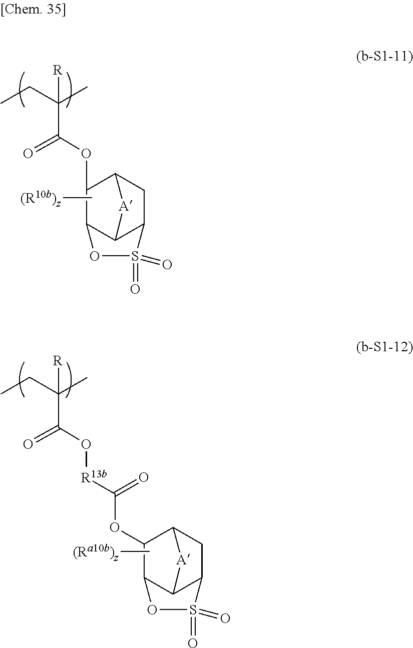

(In the formulae, R, A′, R^{10b}, z and R^{13b} are each the same as the above.)

In the formula (b-S1-11), A′ is preferably a methylene group, an oxygen atom (—O—) or a sulfur atom (—S—).

As R^{13b}, preferred is a linear or branched alkylene group or a divalent linking group including an oxygen atom. Examples of the linear or branched alkylene group and the divalent linking group including an oxygen atom in R^{13b} include those similar to the aforementioned linear or branched alkylene group and the aforementioned divalent linking group including an oxygen atom, respectively.

As the constituent unit represented by the formula (b-S1-12), particularly preferred is one represented by the following formula (b-S1-12a) or (b-S1-12b).

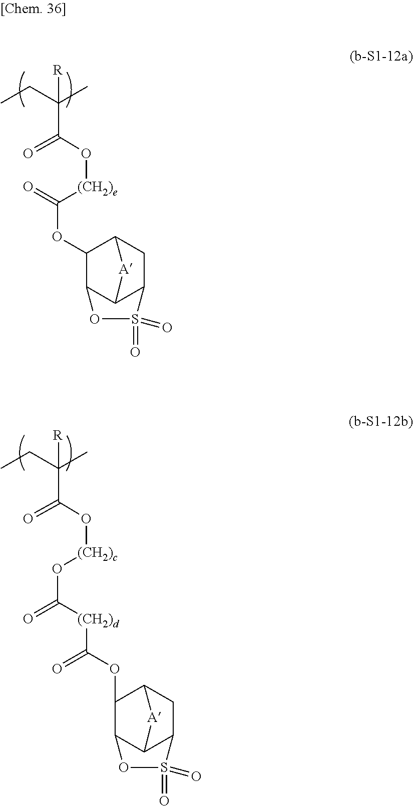

(In the formulae, R and A′ are each the same as the above, and c to e are each independently an integer of 1 or more and 3 or less.)
[Constituent Unit (b-3-L)]

Examples of the constituent unit (b-3-L) include, for example, a constituent unit in which R^{11b} in the aforementioned formula (b-S1) is substituted with a lactone-containing cyclic group. More specifically they include those represented by the following formulae (b-L1) to (b-L5).

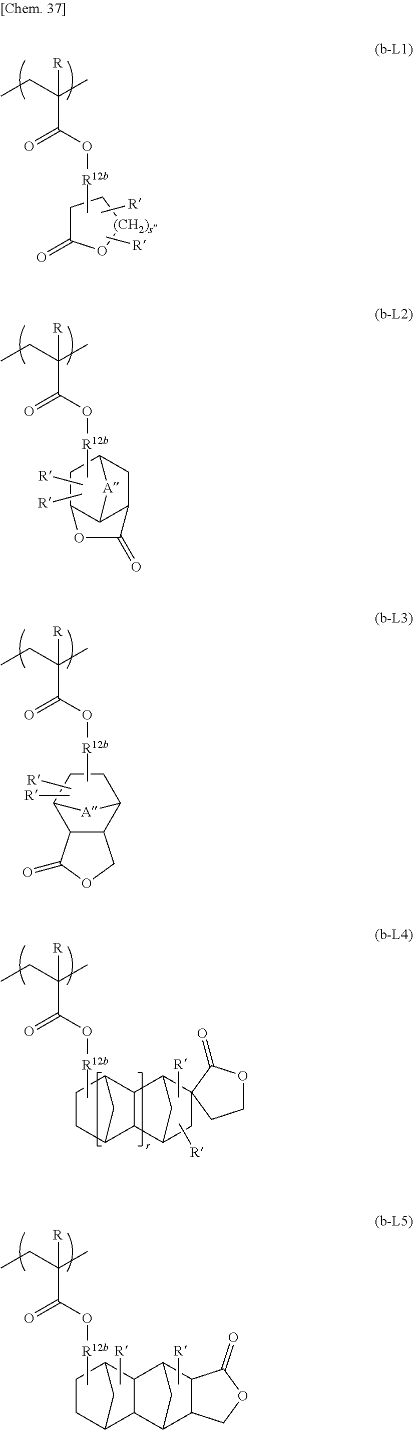

(In the formulae, R represents a hydrogen atom, an alkyl group having 1 or more and 5 or less carbon atoms or a halogenated alkyl group having 1 or more and 5 or less carbon atoms; R′ represents each independently a hydrogen atom, an alkyl group, an alkoxy group, a halogenated alkyl group, a hydroxy group, —COOR″, —OC(═O)R″, a hydroxyalkyl group or a cyano group, and R″ represents a hydrogen atom or an alkyl group; R^{12b} represents a single bond or divalent linking group, and s″ is an integer of 0 or more and 2 or less; A″ represents an alkylene group having 1 or more and 5 or less carbon atoms optionally including an oxygen atom or a sulfur atom, an oxygen atom or a sulfur atom; and r is 0 or 1.)

R in the formulae (b-L1) to (b-L5) is the same as the above. Examples of the alkyl group, the alkoxy group, the halogenated alkyl group, —COOR″, —OC(═O)R″ and the hydroxyalkyl group in R′ include groups similar to those described for the alkyl group, the alkoxy group, the halogenated alkyl group, —COOR″, —OC(═O)R″ and the hydroxyalkyl group recited as a substituent which the —SO₂—containing cyclic group may have, respectively.

R′ is preferably a hydrogen atom in view of easy industrial availability and the like. The alkyl group in R″ may be any of a linear, branched or cyclic chain. In a case where R″ is a linear or branched alkyl group, the number of carbon atoms is preferably 1 or more and 10 or less, and more preferably 1 or more and 5 or less. In a case where R″ is a cyclic alkyl group, the number of carbon atoms is preferably 3 or more and 15 or less, more preferably 4 or more and 12 or less, and most preferably 5 or more and 10 or less. Specific examples include a group in which one or more hydrogen atoms are removed from monocycloalkane and polycycloalkane such as bicycloalkane, tricycloalkane, tetracycloalkane and the like, which may be or not may be substituted with a fluorine atom or a fluorinated alkyl group. Specific examples include a group in which one or more hydrogen atoms are removed from monocycloalkane such as cyclopentane and cyclohexane; and polycycloalkane such as adamantane, norbornane, isobornane, tricyclodecane and tetracyclododecane; and the like. Examples of A″ include groups similar to A′ in the aforementioned formula (3-1). A″ is preferably an alkylene group having 1 to 5 carbon atoms, an oxygen atom (—O—) or a sulfur atom (—S—), more preferably an alkylene group having 1 or more and 5 or less carbon atoms or —O—. As the alkylene group having 1 or more and 5 or less carbon atoms, a methylene group or a dimethylmethylene group is more preferred, and a methylene group is most preferred.

R^{12b} is similar to R^{12b} in the aforementioned formula (b-S1). In the formula (b-L1), s″ is preferably 1 or 2. Below, specific examples of the constituent units represented by the aforementioned formulae (b-L1) to (b-L3) will be illustrated. In each of the following formulae, R^{α} represents a hydrogen atom, a methyl group or a trifluoromethyl group.

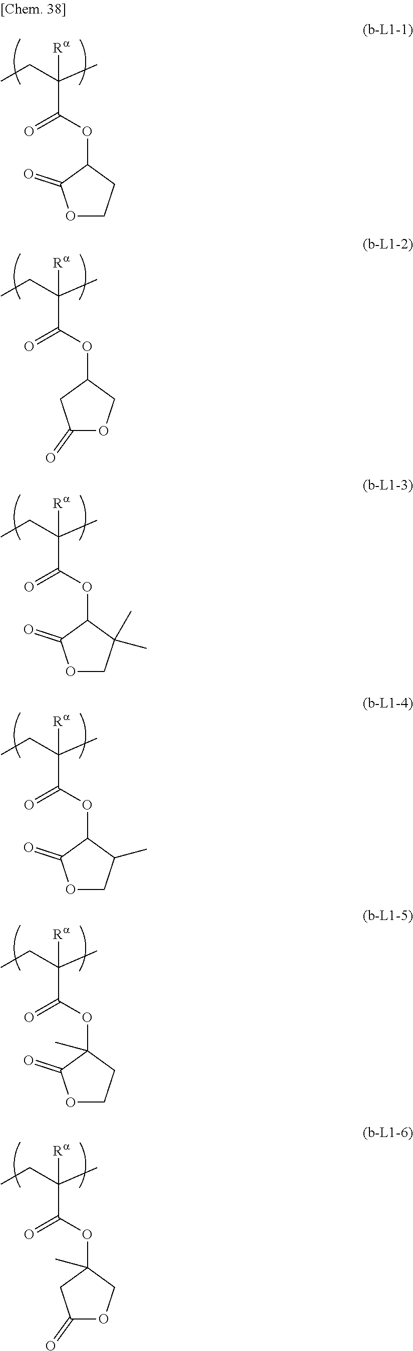

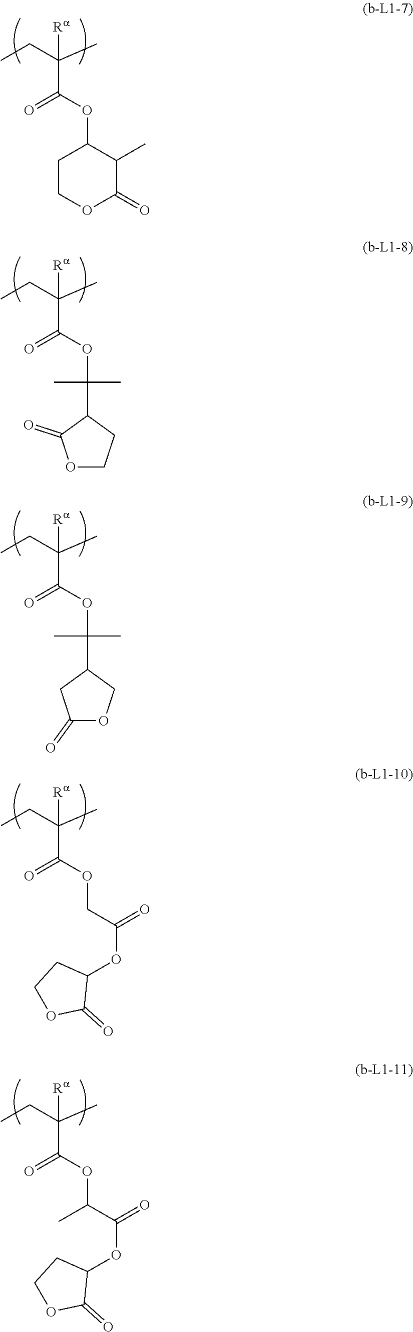

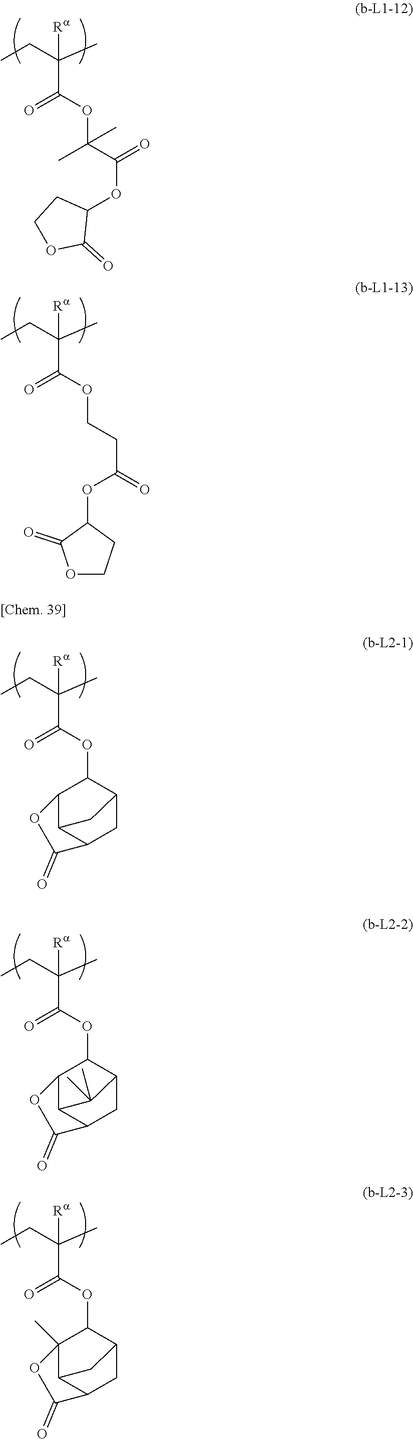

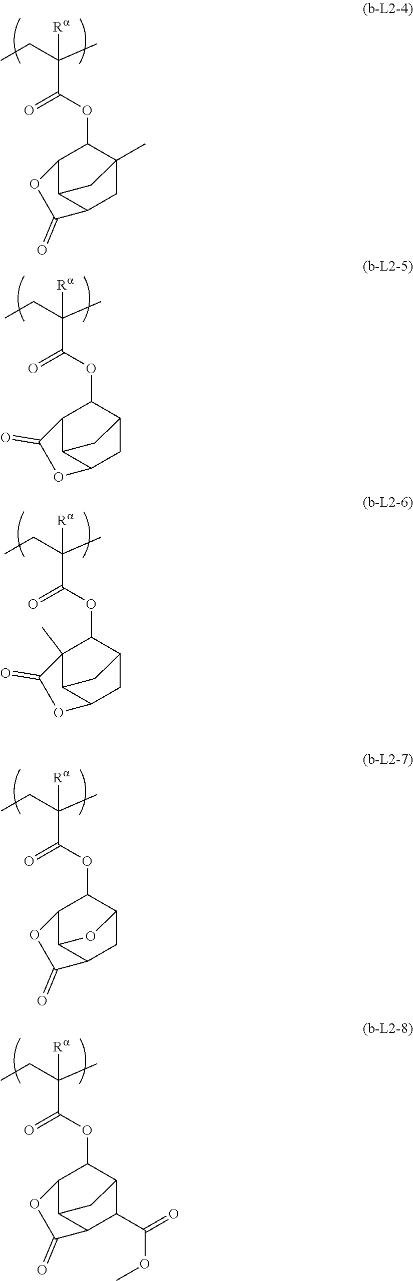

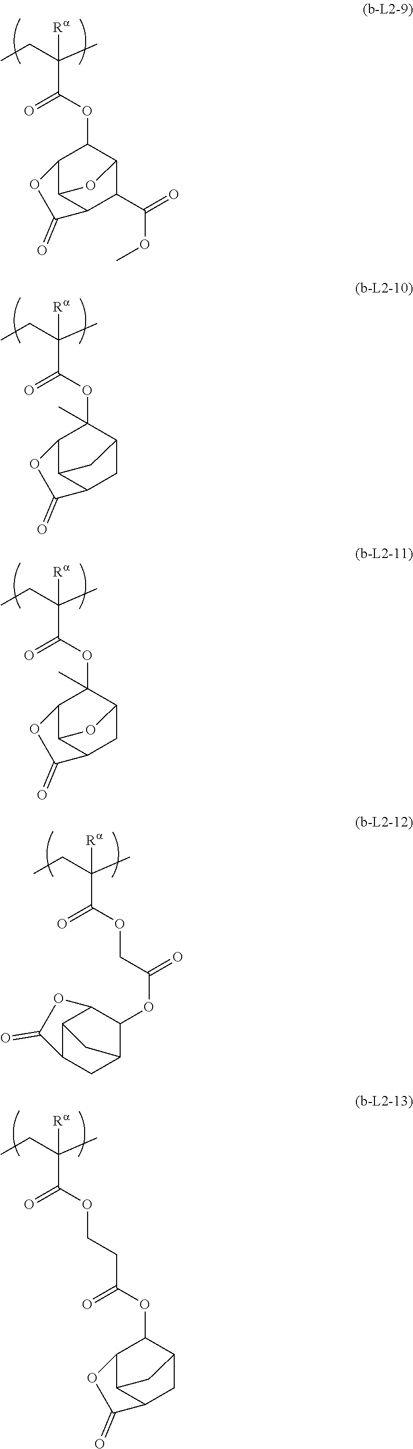

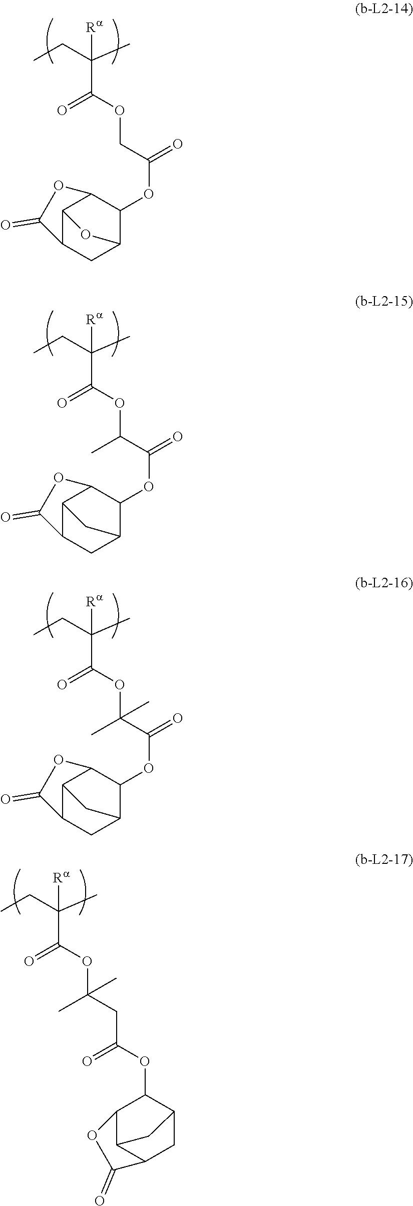

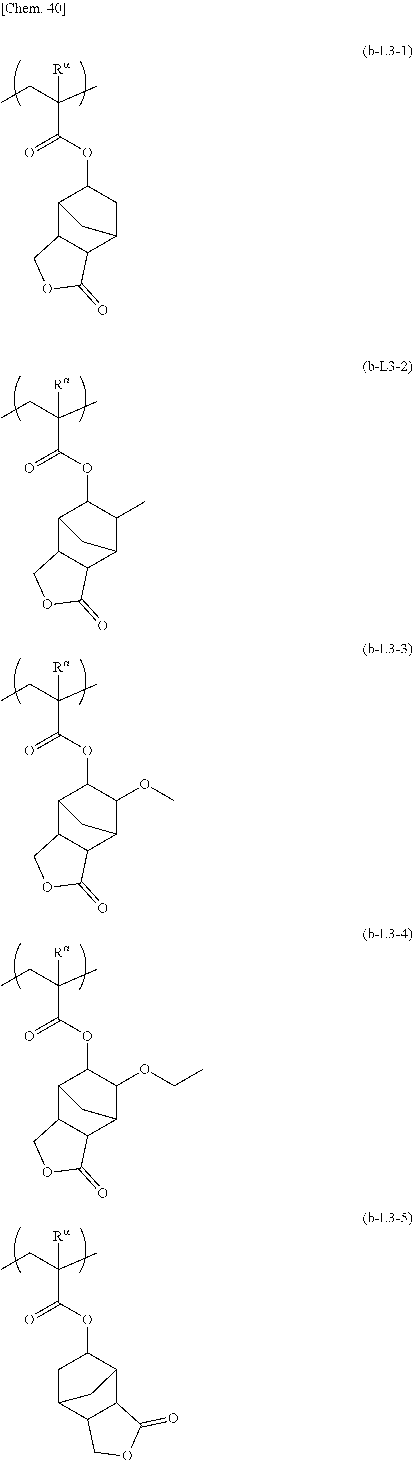

As the constituent unit (b-3-L), at least one selected from the group consisting of the constituent units represented by the aforementioned formulae (b-L1) to (b-L5) is preferred, and at least one selected from the group consisting of the constituent units represented by the formulae (b-L1) to (b-L3) is more preferred, and at least one selected from the group consisting of the constituent units represented by the aforementioned formula (b-L1) or (b-L3) is particularly preferred. Among these, at least one selected from the group consisting of the constituent units represented by the aforementioned formulae (b-L1-1), (b-L1-2), (b-L2-1), (b-L2-7), (b-L2-12), (b-L2-14), (b-L3-1) and (b-L3-5) is preferred.

Further, as the constituent unit (b-3-L), the constituent units represented by following formulae (b-L6) to (b-L7) are also preferred.

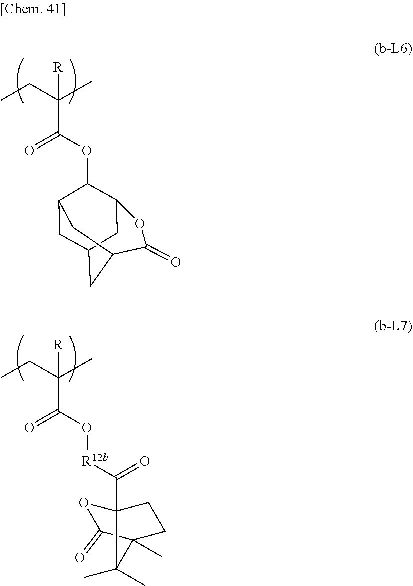

R and R^{12b} in the formulae (b-L6) and (b-L7) are the same as the above.

Further, the acrylic resin (B3) includes constituent units represented by the following formulae (b5) to (b7), having an acid dissociable group, as constituent units that enhance the solubility of the acrylic resin (B3) in alkali under the action of acid.

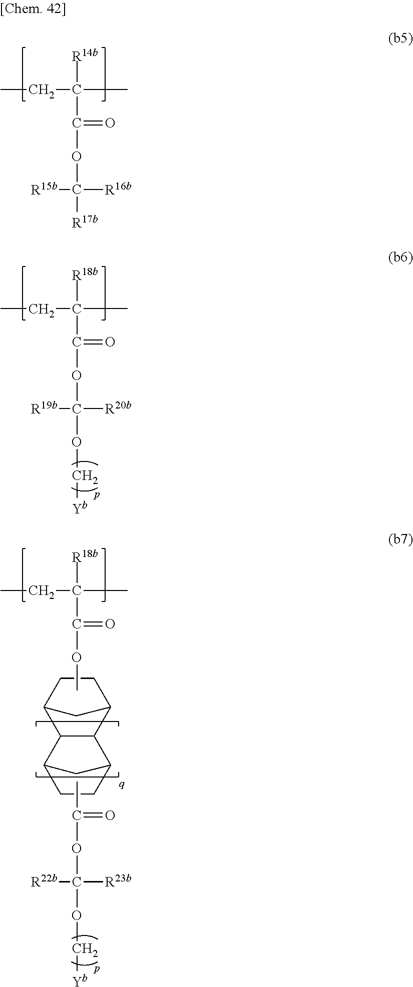

In the above formulae (b5) to (b7), R^{14b} and R^{18b} to R^{23b} each independently represent a hydrogen atom, a linear or branched alkyl group having 1 or more and 6 or less carbon atoms, a fluorine atom, or a linear or branched fluorinated alkyl group having 1 or more and 6 or less carbon atoms; R^{15b} to R^{17b} each independently represent a linear or branched alkyl group having 1 or more and 6 or less carbon atoms, a linear or branched fluorinated alkyl group having 1 or more and 6 or less carbon atoms, or an aliphatic cyclic group having 5 or more and 20 or less carbon atoms, and each independently represent a linear or branched alkyl group having 1 or more and 6 or less carbon atoms, or a linear or branched fluorinated alkyl group having 1 or more and 6 or less carbon atoms; and R^{16b} and R^{17b} may be bonded to each other to form a hydrocarbon ring having 5 or more and 20 or less carbon atoms together with the carbon atom to which both the groups are bonded; Y^{b} represents an optionally substituted aliphatic group or alkyl group; p is an integer of 0 or more and 4 or less; and q is 0 or 1.

Note here that examples of the linear or branched alkyl group include a methyl group, ethyl group, propyl group, isopropyl group, n-butyl group, isobutyl group, tert-butyl group, pentyl group, isopentyl group, neopentyl group, and the like. Furthermore, the fluorinated alkyl group refers to the abovementioned alkyl groups of which the hydrogen atoms are partially or entirely substituted with fluorine atoms. Specific examples of aliphatic cyclic groups include groups obtained by removing one or more hydrogen atoms from monocycloalkanes or polycycloalkanes such as bicycloalkanes, tricycloalkanes, and tetracycloalkanes. Specifically, groups obtained by removing one hydrogen atom from a monocycloalkane such as cyclopentane, cyclohexane, cycloheptane, or cyclooctane, or a polycycloalkane such as adamantane, norbornane, isobornane, tricyclodecane, or tetracyclododecane may be mentioned. In particular, groups obtained by removing one hydrogen atom from cyclohexane or adamantane (which may further be substituted) are preferred.

When R^{16b} and R^{17b} do not combine with each other to form a hydrocarbon ring, the above R^{15b}, R^{16b}, and R^{17b} preferably represent a linear or branched alkyl group having 2 or more and 4 or less carbon atoms, for example, from the viewpoints of a high contrast and favorable resolution and depth of focus. The above R^{19b}, R^{20b}, R^{22b} and R^{23b} preferably represent a hydrogen atom or a methyl group.

The above R^{16b} and R^{17b} may form an aliphatic cyclic group having 5 or more and 20 or less carbon atoms together with a carbon atom to which the both are attached. Specific examples of such an alicyclic group are the groups of monocycloalkanes and polycycloalkanes such as bicycloalkanes, tricycloalkanes and tetracycloalkanes from which one or more hydrogen atoms are removed. Specific examples thereof are the groups of monocycloalkanes such as cyclopentane, cyclohexane, cycloheptane and cyclooctane and polycycloalkanes such as adamantane, norbornane, isobornane, tricyclodecane and tetracyclododecane from which one or more hydrogen atoms are removed. Particularly preferable are the groups of cyclohexane and adamantane from which one or more hydrogen atoms are removed (that may further have a substituent).

Further, in a case where an aliphatic cyclic group to be formed with the above R^{16b} and R^{17b} has a substituent on the ring skeleton thereof, examples of the substituent include a polar group such as a hydroxy group, a carboxyl group, a cyano group and an oxygen atom (═O), and a linear or branched alkyl group having 1 or more and 4 or less carbon atoms. As the polar group, an oxygen atom (═O) is particularly preferred.

The above Y^{b} is an alicyclic group or an alkyl group; and examples thereof are the groups of monocycloalkanes and polycycloalkanes such as bicycloalkanes, tricycloalkanes and tetracycloalkanes from which one or more hydrogen atoms are removed. Specific examples thereof are the groups of monocycloalkanes such as cyclopentane, cyclohexane, cycloheptane and cyclooctane, and polycycloalkanes such as adamantane, norbornane, isobornane, tricyclodecane and tetracyclododecane from which one or more hydrogen atoms are removed. Particularly preferable is the group of adamantane from which one or more hydrogen atoms are removed (that may further have a substituent).

When the alicyclic group of the above Y^{b} has a substituent on the ring skeleton, the substituent is exemplified by polar groups such as a hydroxy group, carboxyl group, cyano group and oxygen atom (═O), and linear or branched alkyl groups having 1 or more and 4 or less carbon atoms. The polar group is preferably an oxygen atom (═O) in particular.

When Y^{b} is an alkyl group, it is preferably a linear or branched alkyl group having 1 or more and 20 or less carbon atoms, and more preferably 6 or more and 15 or less carbon atoms. The alkyl group is an alkoxyalkyl group particularly preferable. Examples of such an alkoxyalkyl group include a 1-methoxyethyl group, 1-ethoxyethyl group, 1-n-propoxyethyl group, 1-isopropoxyethyl group, 1-n-butoxyethyl group, 1-isobutoxyethyl group, 1-tert-butoxyethyl group, 1-methoxypropyl group, 1-ethoxypropyl group, 1-methoxy-1-methylethyl group, 1-ethoxy-1-methylethyl group, and the like.

Preferable specific examples of the constituent unit represented by the above formula (b5) include constituent units represented by the following formulae (b5-1) to (b5-33).

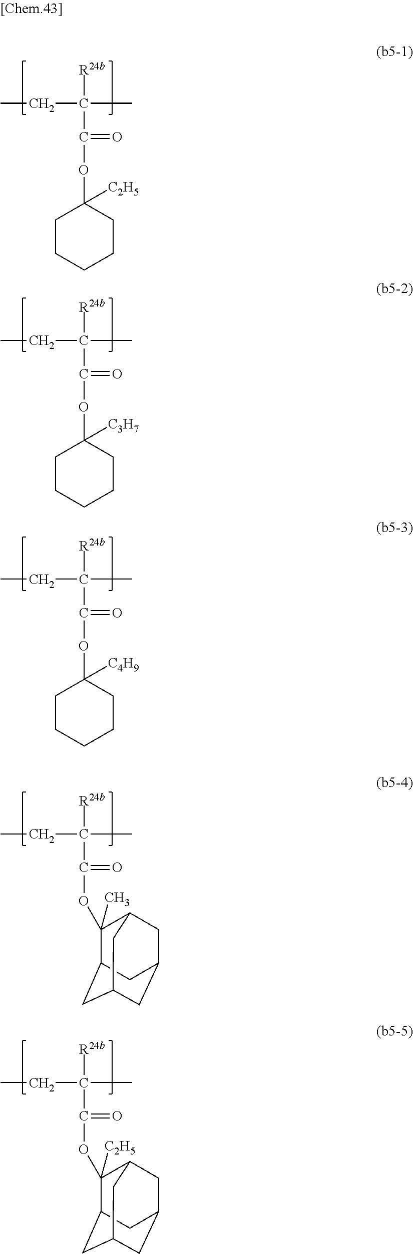

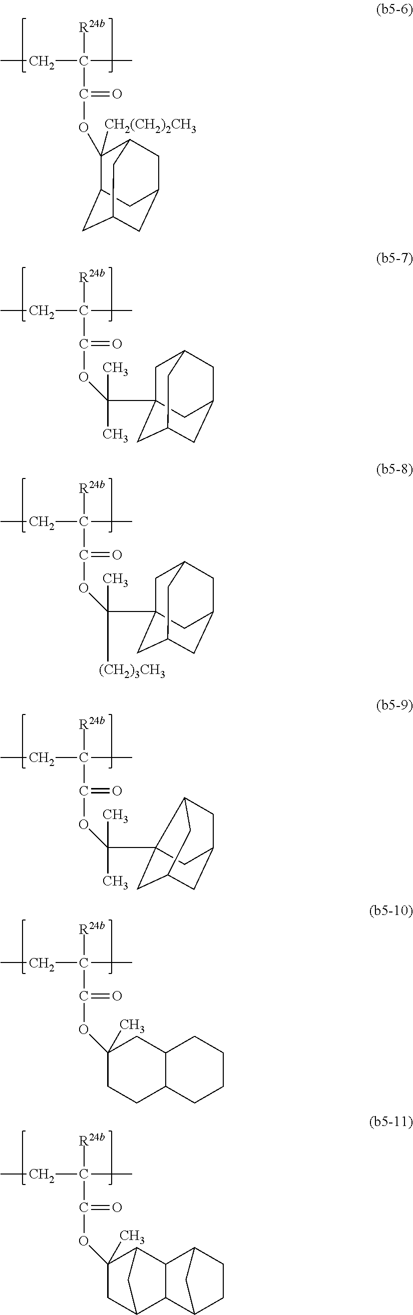

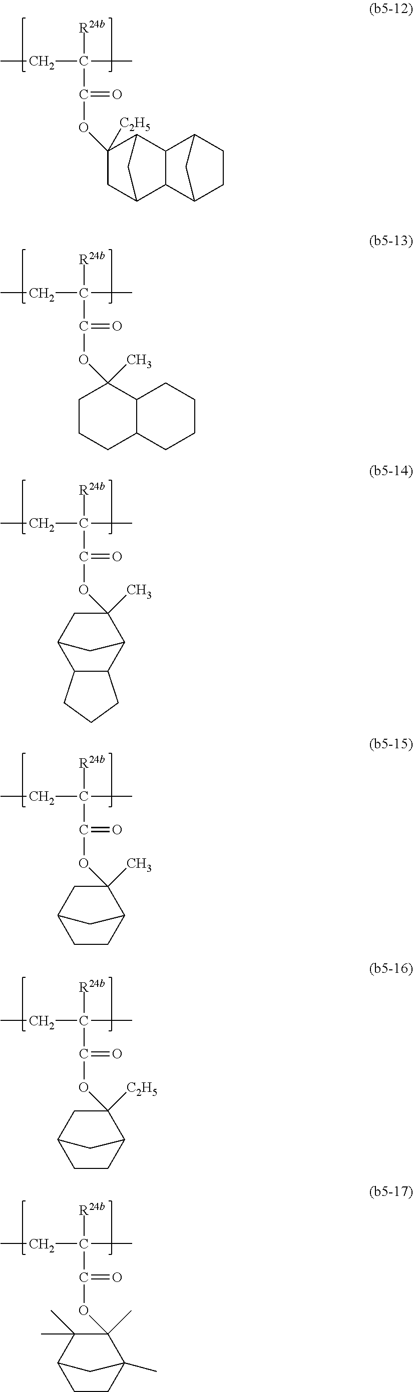

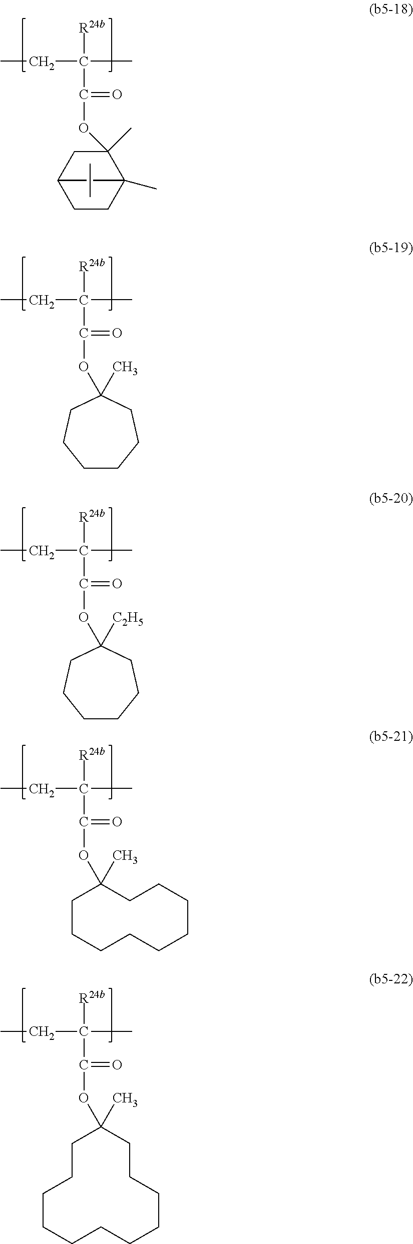

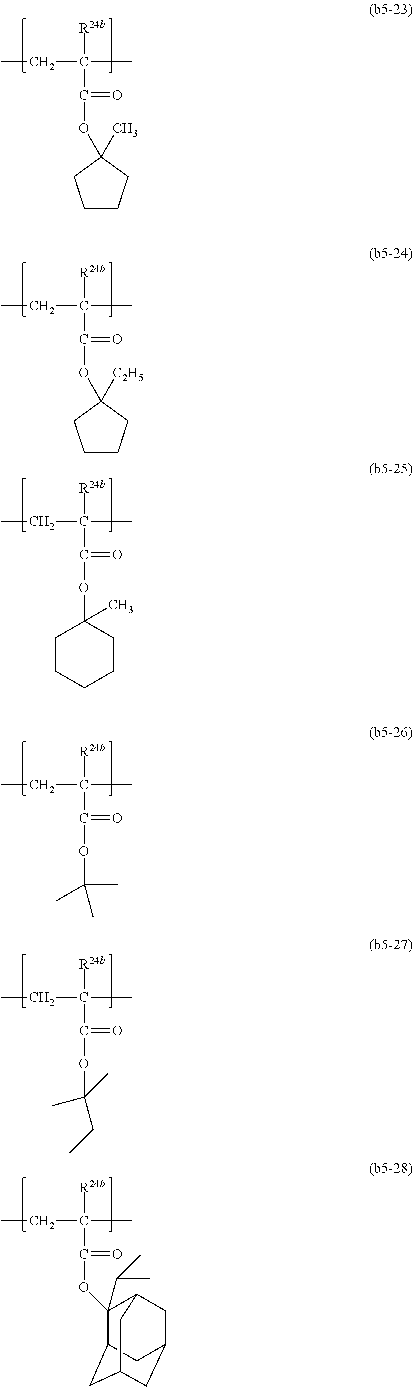

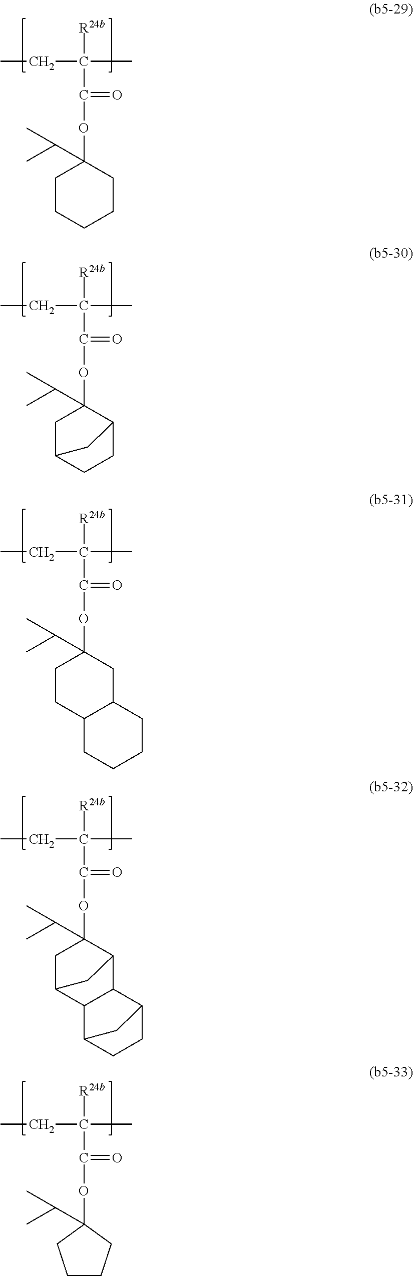

In the above formulae (b5-1) to (b5-33), R^{24b} represents a hydrogen atom or a methyl group.

Preferable specific examples of the constituent unit represented by the above formula (b6) include constituent units represented by the following formulae (b6-1) to (b6-26).

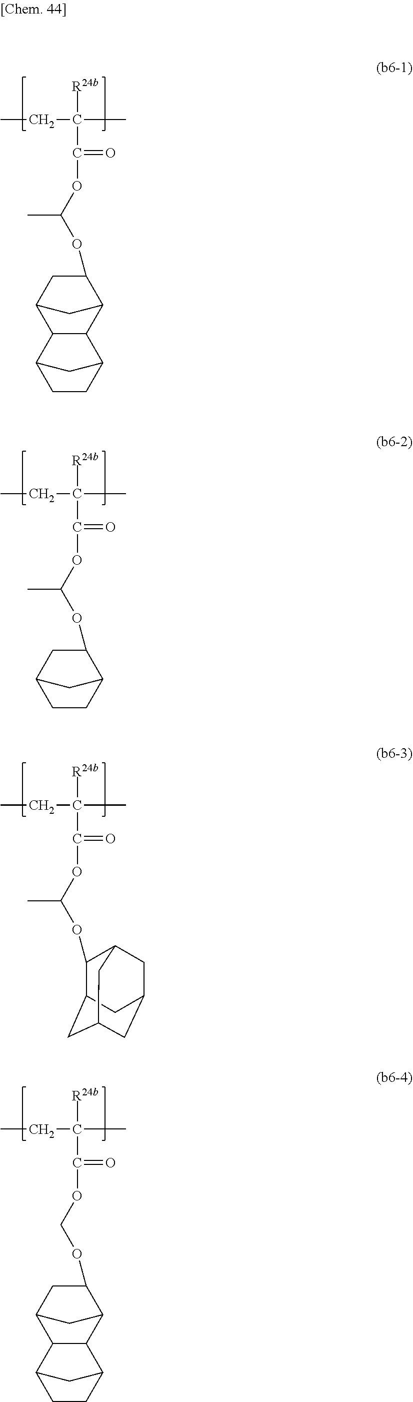

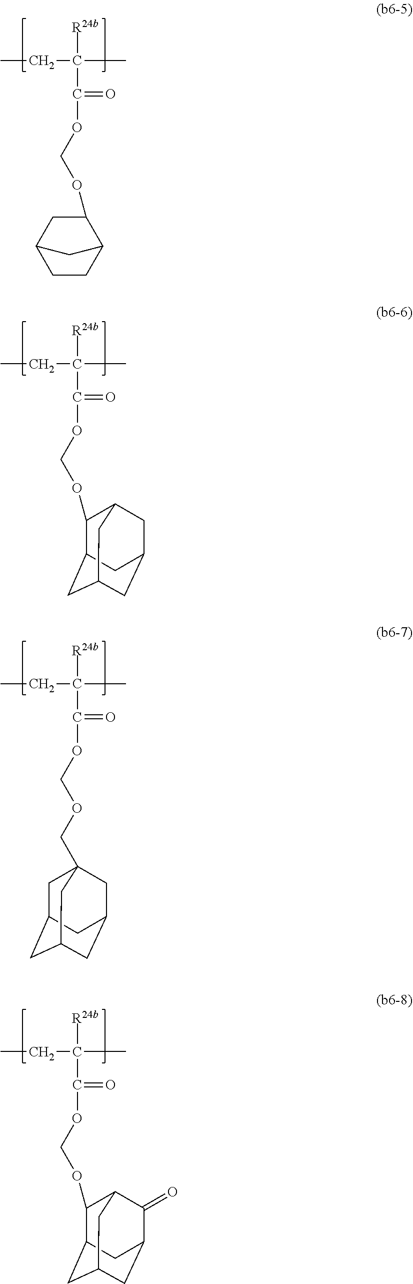

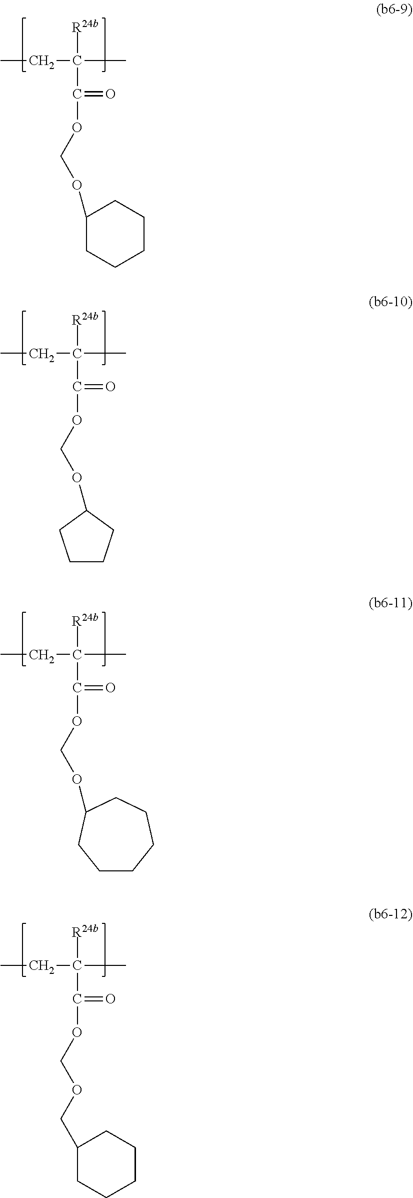

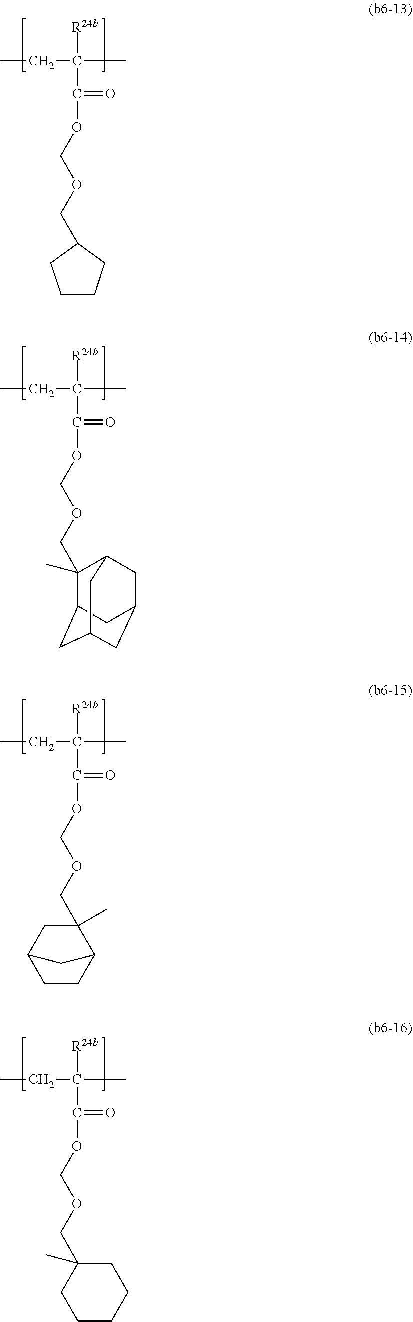

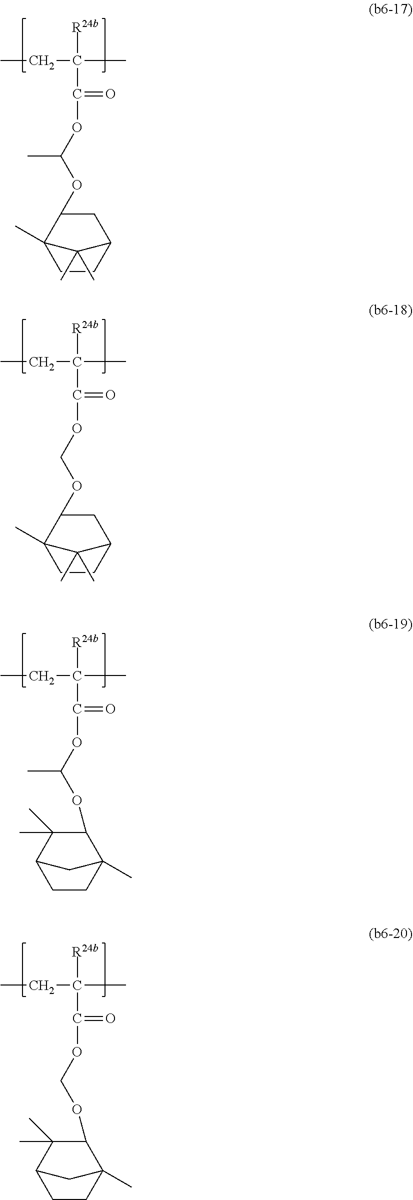

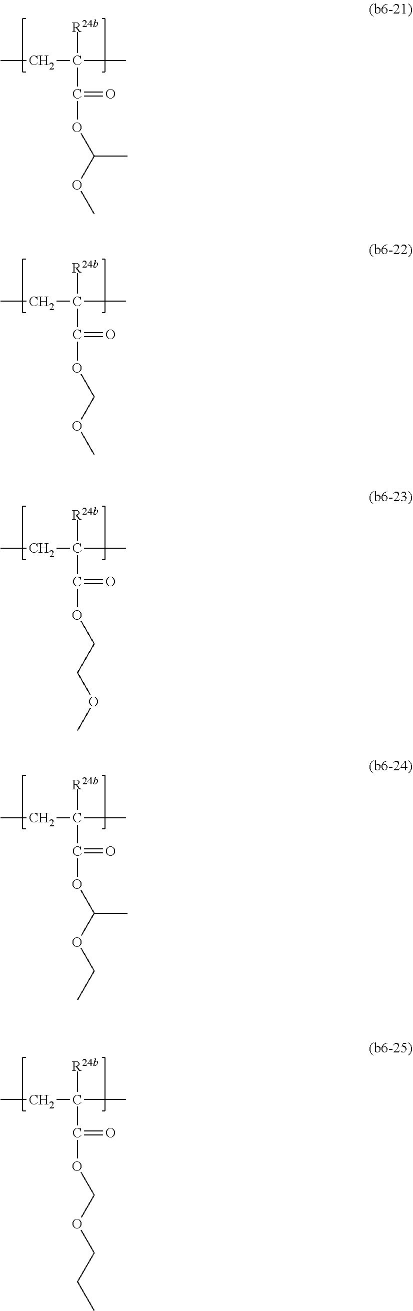

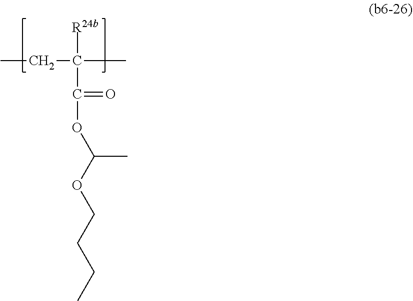

In the above formulae (b6-1) to (b6-26), R^{24b} represents a hydrogen atom or a methyl group.

Preferable specific examples of the constituent unit represented by the above formula (b7) include constituent units represented by the following formulae (b7-1) to (b7-15).

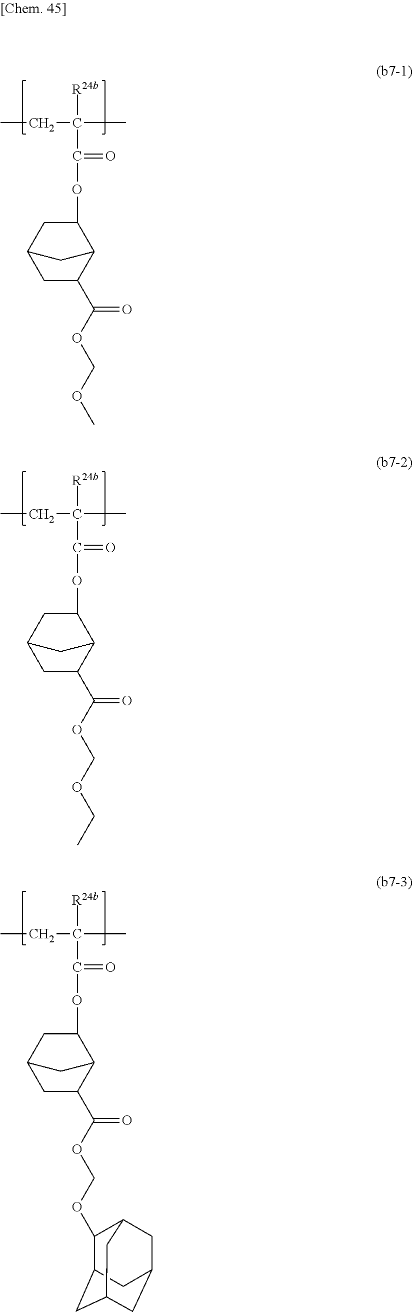

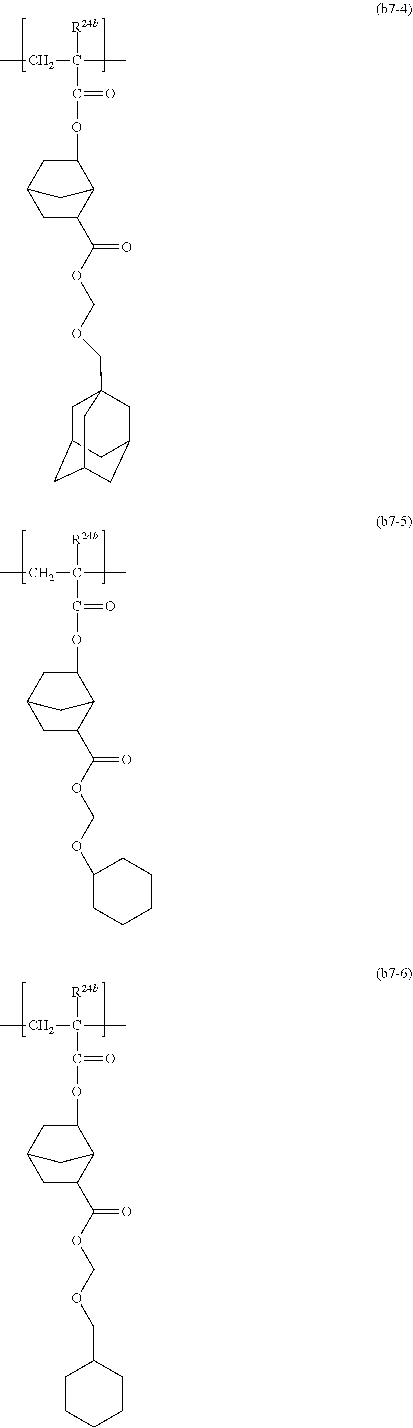

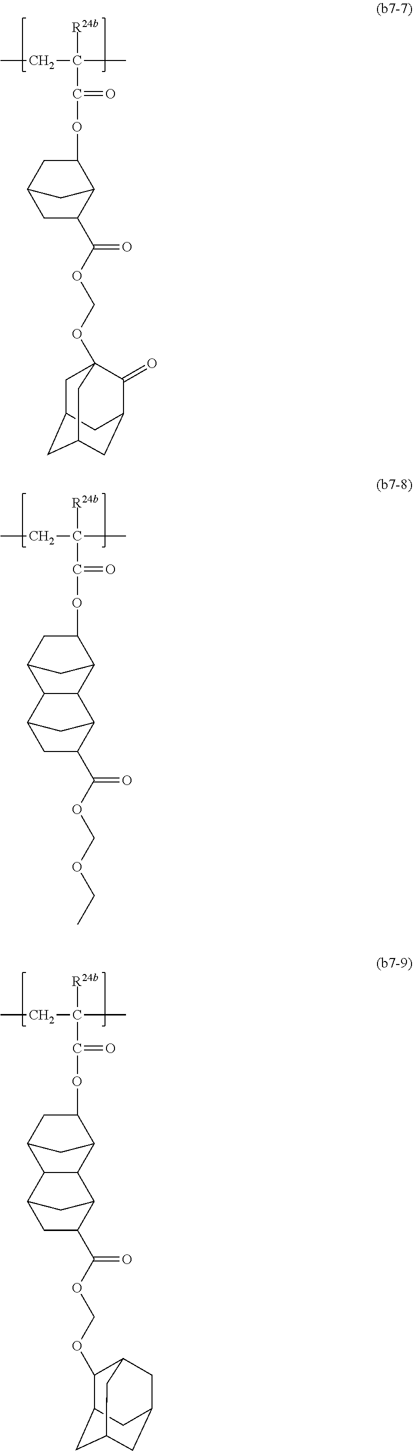

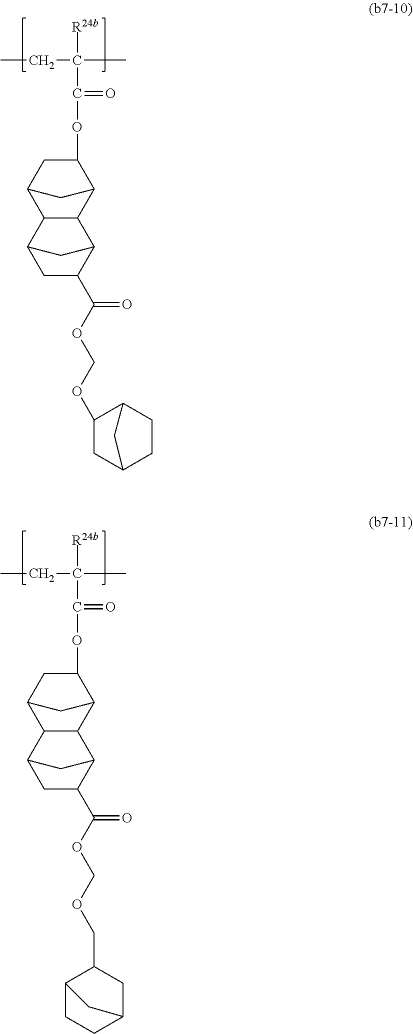

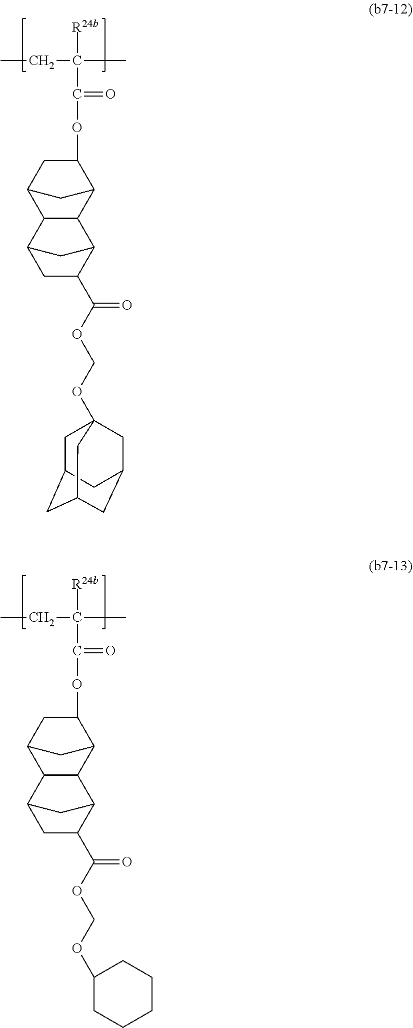

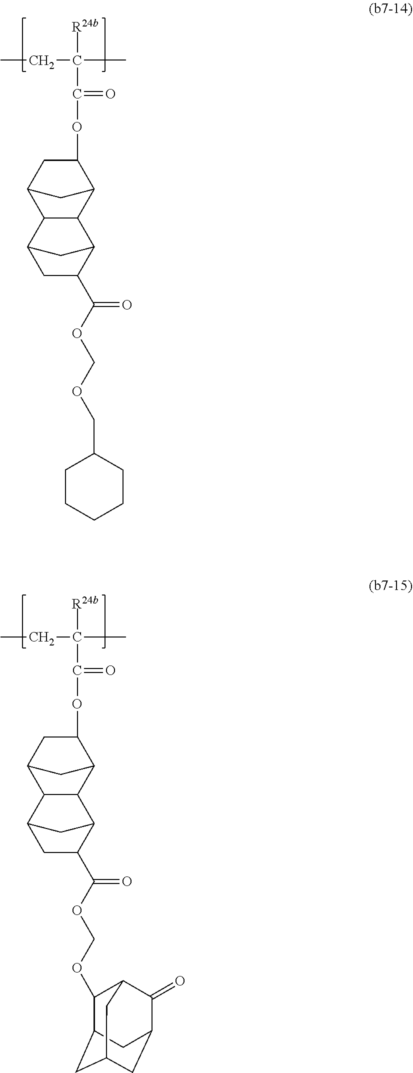

In the above formulae (b7-1) to (b7-15), R^{24b} represents a hydrogen atom or a methyl group.

Among the constituent units represented by the formulae (b5) to (b7) described above, those represented by the formula (b6) are preferred in that they can be easily synthesized and relatively easily sensitized. Further, among the constituent units represented by the formula (b6), those in which Y^{b} is an alkyl group are preferred, and those in which one of R^{19b} and R^{20b} or both R^{19b} and R^{20b} are alkyl groups are preferred.

Further, the acrylic resin (B3) is preferably a resin including a copolymer including a constituent unit derived from a polymerizable compound having an ether bond together with a constituent unit represented by the above formulae (b5) to (b7).

Illustrative examples of the polymerizable compound having an ether bond include radical polymerizable compounds such as (meth)acrylic acid derivatives having an ether bond and an ester bond, and specific examples thereof include 2-methoxyethyl (meth)acrylate, 2-ethoxyethyl (meth)acrylate, methoxytriethylene glycol (meth) acrylate, 3-methoxybutyl (meth) acrylate, ethylcarbitol (meth) acrylate, phenoxypolyethylene glycol (meth) acrylate, methoxypolyethylene glycol (meth)acrylate, methoxypolypropylene glycol (meth)acrylate, tetrahydrofurfuryl (meth)acrylate, and the like. Also, the above polymerizable compound having an ether bond is preferably, 2-methoxyethyl (meth)acrylate, 2-ethoxyethyl (meth)acrylate, or methoxytriethylene glycol (meth)acrylate. These polymerizable compounds may be used alone, or in combinations of two or more thereof.

Furthermore, the acrylic resin (B3) may include another polymerizable compound as a constituent unit in order to moderately control physical or chemical properties. The polymerizable compound is exemplified by conventional radical polymerizable compounds and anion polymerizable compounds.

Examples of the polymerizable compound include monocarboxylic acids such as acrylic acid, methacrylic acid and crotonic acid; dicarboxylic acids such as maleic acid, fumaric acid and itaconic acid; methacrylic acid derivatives having a carboxyl group and an ester bond such as 2-methacryloyloxyethyl succinic acid, 2-methacryloyloxyethyl maleic acid, 2-methacryloyloxyethyl phthalic acid, and 2-methacryloyloxyethyl hexahydrophthalic acid; (meth)acrylic acid alkyl esters such as methyl(meth)acrylate, ethyl(meth)acrylate, butyl(meth)acrylate and cyclohexyl(meth)acrylate; (meth)acrylic acid hydroxyalkyl esters such as 2-hydroxyethyl (meth)acrylate and 2-hydroxypropyl (meth)acrylate; (meth)acrylic acid aryl esters such as phenyl (meth)acrylate and benzyl (meth)acrylate; dicarboxylic acid diesters such as diethyl maleate and dibutyl fumarate; vinyl group-containing aromatic compounds such as styrene, α-methylstyrene, chlorostyrene, chloromethylstyrene, vinyltoluene, hydroxystyrene, α-methylhydroxystyrene and α-ethylhydroxystyrene; vinyl group-containing aliphatic compounds such as vinyl acetate; conjugated diolefins such as butadiene and isoprene; nitrile group-containing polymerizable compounds such as acrylonitrile and methacrylonitrile; chlorine-containing polymerizable compounds such as vinyl chloride and vinylidene chloride; amide bond-containing polymerizable compounds such as acrylamide and methacrylamide; and the like.

As described above, the acrylic resin (B3) may include a constituent unit derived from a polymerizable compound having a carboxy group such as the above monocarboxylic acids and dicarboxylic acids. However, it is preferable that the acrylic resin (B3) does not substantially include a constituent unit derived from a polymerizable compound having a carboxyl group, since a resist pattern including a nonresist portion having a favorable rectangular sectional shape can easily be formed. Specifically, the proportion of a constituent unit derived from a polymerizable compound having a carboxyl group in the acrylic resin (B3) is preferably 20% by mass or less, more preferably 15% by mass or less, and particularly preferably 5% by mass or less. In acrylic resin (B3), acrylic resin including a relatively large amount of constituent unit derived from a polymerizable compound having a carboxy group is preferably used in combination with an acrylic resin that includes only a small amount of constituent unit derived from a polymerizable compound having a carboxy group or does not include this constituent unit.

Furthermore, examples of the polymerizable compound include (meth)acrylic acid esters having a non-acid-dissociable aliphatic polycyclic group, and vinyl group-containing aromatic compounds and the like. As the non-acid-dissociable aliphatic polycyclic group, particularly, a tricyclodecanyl group, an adamantyl group, a tetracyclododecanyl group, an isobornyl group, a norbornyl group, and the like are preferred in view of easy industrial availability and the like. These aliphatic polycyclic groups may have a linear or branched alkyl group having 1 or more and 5 or less carbon atoms as a substituent.

Specific examples of the (meth)acrylic acid esters having a non-acid-dissociable aliphatic polycyclic group include those having structures represented by the following formulae (b8-1) to (b8-5).

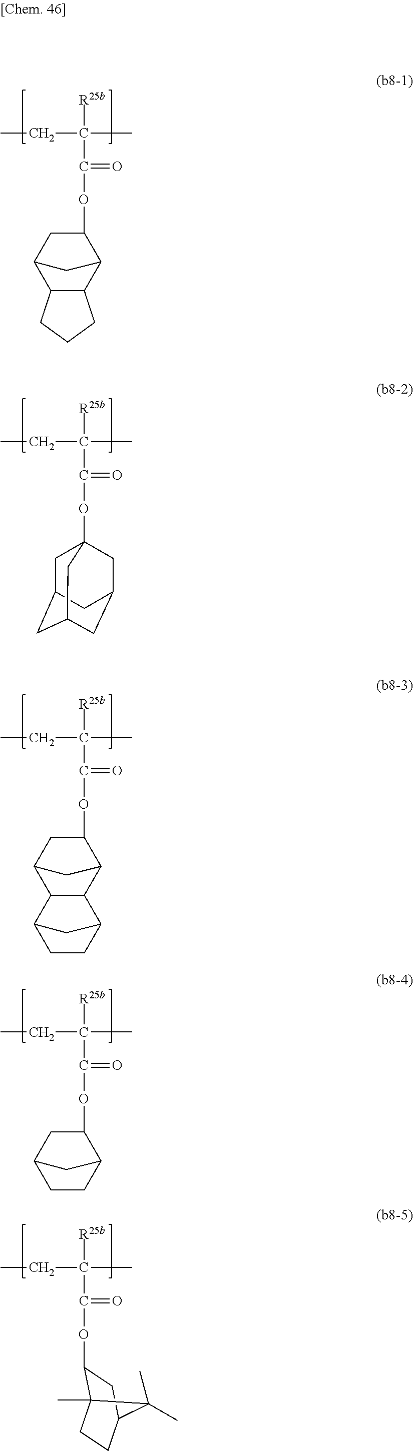

In formulae (b8-1) to (b8-5), R^{25b} represents a hydrogen atom or a methyl group.

When the acrylic resin (B3) includes the constituent unit (b-3) including a —SO₂-containing cyclic group or a lactone-containing cyclic group, the content of the constituent unit (b-3) in the acrylic resin (B3) is preferably 5% by mass or more, more preferably 10% by mass or more, and particularly preferably 10% by mass or more and 50% by mass or less, and most preferably 10% by mass or more and 30% by mass or less. In a case where the photosensitive resin composition includes the constituent unit (b-3) having the above-mentioned range of amount, both good developing property and a good pattern shape can be easily achieved simultaneously.

Further, in the acrylic resin (B3), a constituent unit represented by the aforementioned formulae (b5) to (b7) is preferably included in an amount of 5% by mass or more, more preferably 10% by mass or more, and particularly preferably 10% by mass or more and 50% by mass or less.

The acrylic resin (B3) preferably includes the above constituent unit derived from a polymerizable compound having an ether bond. The content of the constituent unit derived from a polymerizable compound having an ether bond in the acrylic resin (B3) is preferably 0% by mass or more and 50% by mass or less, more preferably 5% by mass or more and 30% by mass or less.

The acrylic resin (B3) preferably includes the above constituent unit derived from (meth)acrylic acid esters having a non-acid-dissociable aliphatic polycyclic group. The content of the constituent unit derived from (meth)acrylic acid esters having a non-acid-dissociable aliphatic polycyclic group in the acrylic resin (B3) is preferably 0% by mass or more and 50% by mass or less, and more preferably 5% by mass or more and 30% by mass or less.

As the resin (B), an acrylic resin other than the acrylic resin (B3) described above may also be used, as long as the photosensitive resin composition contains a predetermined amount of the acrylic resin (B3). As such an acrylic resin other than the acrylic resin (B3), there is no particular limitation as long as it is a resin containing a constitutional unit represented by the aforementioned formulas (b5) to (b7).

The mass-average molecular weight of the resin (B) described above in terms of polystyrene is preferably 10000 or more and 600000 or less, more preferably 20000 or more and 400000 or less, and most preferably 30000 or more and 300000 or less. Such a mass-average molecular weight within these ranges allows the photosensitive layer to maintain sufficient strength without reducing detachability from a substrate, and can prevent a swelled profile and crack generation when plating.

It is also preferred that the resin (B) has a dispersivity of 1.05 or more. Dispersivity herein indicates a value of a mass average molecular weight divided by a number average molecular weight. Such a dispersivity in the range described above can avoid problems with respect to stress resistance on intended plating or possible swelling of metal layers resulting from the plating process.

The content of the resin (B) is preferably 5% by mass or more and 70% by mass or less, with respect to the total solid content of the photosensitive resin composition.

### <Alkali-Soluble Resin (D)>

It is preferable that the photosensitive resin composition further contains an alkali-soluble resin (D) in order to improve alkali-solubility. The alkali-soluble resin as referred to herein may be determined as follows. A resin solution having a resin concentration of 20% by mass (solvent: propylene glycol monomethyl ether acetate) is used to form a resin film having a thickness of 1 μm on a substrate, and immersed in an aqueous 2.38% by mass TMAH (tetramethylammonium hydroxide) solution for 1 minute. When the resin was dissolved in an amount of 0.01 μm or more, the resin is defined as being alkali soluble, wherein the alkali-soluble resin is not the component (B) described above (typically, the resin is defined as resin whose alkali-solubility does not substantially change even under action of acid). As the alkali-soluble resin (D), preferably, for example, at least one selected from the group consisting of a novolac resin (D1), a polyhydroxystyrene resin (D2), and an acrylic resin (D3), can be used.

### [Novolak Resin (D1)]

A novolak resin is obtained by addition condensation of, for example, aromatic compounds having a phenolic hydroxy group (hereinafter, merely referred to as “phenols”) and aldehydes in the presence of an acid catalyst.

Examples of the above phenols include phenol, o-cresol, m-cresol, p-cresol, o-ethylphenol, m-ethylphenol, p-ethylphenol, o-butylphenol, m-butylphenol, p-butylphenol, 2,3-xylenol, 2,4-xylenol, 2,5-xylenol, 2,6-xylenol, 3,4-xylenol, 3,5-xylenol, 2,3,5-trimethyl phenol, 3,4,5-trimethyl phenol, p-phenylphenol, resorcinol, hydroquinone, hydroquinone monomethyl ether, pyrogallol, phloroglycinol, hydroxydiphenyl, bisphenol A, gallic acid, gallic acid ester, α-naphthol, β-naphthol, and the like. Examples of the above aldehydes include formaldehyde, furfural, benzaldehyde, nitrobenzaldehyde, acetaldehyde, and the like. The catalyst used in the addition condensation reaction is not particularly limited, and examples thereof include hydrochloric acid, nitric acid, sulfuric acid, formic acid, oxalic acid, acetic acid, etc., for acid catalyst.

The flexibility of the novolak resins can be enhanced more when o-cresol is used, a hydrogen atom of a hydroxyl group in the resins is substituted with other substituents, or bulky aldehydes are used.

The mass average molecular weight of novolac resin (D1) is not particularly limited as long as the purpose of the present invention is not impaired, but the mass average molecular weight is preferably 1,000 or more and 50,000 or less.

### [Polyhydroxystyrene Resin (D2)]

The hydroxystyrene-based compound to constitute the polyhydroxystyrene resin (D2) is exemplified by p-hydroxystyrene, α-methylhydroxystyrene, α-ethylhydroxystyrene, and the like. Furthermore, the polyhydroxystyrene resin (D2) is preferably made to give a copolymer with a styrene resin. Examples of the styrene compound to constitute such a styrene resin include styrene, chlorostyrene, chloromethylstyrene, vinyltoluene, α-methylstyrene, and the like.

The mass average molecular weight of the polyhydroxystyrene resin (D2) is not particularly limited as long as the purpose of the present invention is not impaired, but the mass average molecular weight is preferably 1,000 or more and 50,000 or less.

### [Acrylic Resin (D3)]

It is preferable that the acrylic resin (D3) includes a constituent unit derived from a polymerizable compound having an ether bond and a constituent unit derived from a polymerizable compound having a carboxyl group.

Examples of the above polymerizable compound having an ether bond include (meth)acrylic acid derivatives having an ether bond and an ester bond such as 2-methoxyethyl (meth)acrylate, methoxytriethylene glycol (meth)acrylate, 3-methoxybutyl (meth) acrylate, ethylcarbitol (meth) acrylate, phenoxypolyethylene glycol (meth) acrylate, methoxypolypropylene glycol (meth) acrylate, tetrahydrofurfuryl (meth)acrylate, and the like. The above polymerizable compound having an ether bond is preferably, 2-methoxyethyl acrylate, and methoxytriethylene glycol acrylate. These polymerizable compounds may be used alone, or in combinations of two or more.

Examples of the above polymerizable compound having a carboxy group include monocarboxylic acids such as acrylic acid, methacrylic acid and crotonic acid; dicarboxylic acids such as maleic acid, fumaric acid and itaconic acid; compounds having a carboxy group and an ester bond such as 2-methacryloyloxyethyl succinic acid, 2-methacryloyloxyethyl maleic acid, 2-methacryloyloxyethyl phthalic acid, 2-methacryloyloxyethyl hexahydrophthalic acid and the like. The above polymerizable compound having a carboxy group is preferably, acrylic acid and methacrylic acid. These polymerizable compounds may be used alone, or in combinations of two or more thereof.

The mass average molecular weight of the acrylic resin (D3) is not particularly limited as long as the purpose of the present invention is not impaired, but the mass average molecular weight is preferably 50,000 or more and 800,000 or less.

The content of the alkali-soluble resin (D) is preferably 3 parts by mass or more and 70 parts by mass or less, and more preferably 5 parts by mass or more and 50 parts by mass or less when the total solid content is 100 parts by mass. By setting the content of the alkali-soluble resin (D) to the range described above, there is a tendency for alkali-solubility.

### <Sulfur-Containing Compound (E)>

When the photosensitive resin composition is used for pattern formation on a metal substrate, the photosensitive resin composition preferably includes a sulfur-containing compound (E). The sulfur-containing compound (E) is a compound including a sulfur atom that can coordinate with metal. Note here that in a compound that can generate two or more tautomers, at least one tautomer includes a sulfur atom that coordinates with metal constituting a surface of the metal substrate, the compound corresponds to a sulfur-containing compound. When a resist pattern serving as a template for plating is formed on a surface made of metal such as Cu, defectives such as footing having a cross-sectional shape easily occur. However, when the photosensitive resin composition includes a sulfur-containing compound (E), even when a resist pattern is formed on a surface made of metal in a substrate, defectives such as footing having a cross-sectional shape is easily suppressed. Note here that the “footing” is a phenomenon in which the width of the bottom becomes narrower than that of the top in a nonresist section due to protrusion of a resist section toward the nonresist section in the vicinity of the contacting surface between the substrate surface and the resist pattern. When the photosensitive resin composition is used for pattern formation on a substrate other than a metal substrate, it is not particularly necessary that the photosensitive resin composition contains a sulfur-containing compound. When the photosensitive resin composition is used for pattern formation on a substrate other than a metal substrate, it is preferable that the photosensitive resin composition does not contain a sulfur-containing compound (E) from the viewpoint of easy manufacturing of the photosensitive resin composition due to reduction in the number of components of the photosensitive resin composition, and of reduced manufacturing costs. Note that there are no particular problems caused by the presence of the sulfur-containing compounds (E) in the photosensitive resin composition used for pattern formation on a substrate other than a metal substrate.

The sulfur atom that can coordinate with metal is included in a sulfur-containing compound as, for example, a mercapto group (—SH), a thiocarboxy group (—CO—SH), a dithiocarboxy group (—CS—SH), a thiocarbonyl group (—CS—), and the like. From the viewpoint of easiness in coordinating with metal and being excellent in suppressing footing, the sulfur-containing compound preferably includes a mercapto group.

Preferable examples of the sulfur-containing compound having a mercapto group include compounds represented by the following formula (e1).

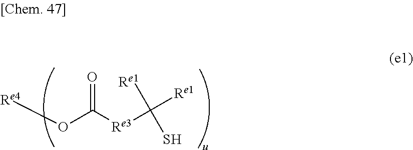

(In the formula, R^{e1} and R^{e2} each independently represent a hydrogen atom or an alkyl group, R^{e3} represents a single bond or an alkylene group, R^{e4} represents a u-valence aliphatic group optionally including an atom other than carbon, and u is an integer of 2 or more and 4 or less.)

R^{e1} and R^{e2} are an alkyl group, the alkyl group may be linear or branched, and is preferably linear. When R^{e1} and R^{e2} are an alkyl group, the number of carbon atoms of the alkyl group is not particularly limited within a range where the objects of the present invention are not impaired. The number of carbon atoms of the alkyl group is preferably 1 or more and 4 or less, particularly preferably 1 or 2, and the most preferably 1. As the combination of R^{e1} and R^{e2}, preferably, one is a hydrogen atom and the other is an alkyl group, and particularly preferably one is a hydrogen atom and the other is a methyl group.

When R^{e3} is an alkylene group, the alkylene group may be linear or branched, and is preferably linear. When R^{e3} is an alkylene group, the number of carbon atoms of the alkylene group is not particularly limited within a range where the objects of the present invention are not impaired. The number of carbon atoms of the alkylene group is preferably 1 or more and 10 or less, more preferably 1 or more and 5 or less, particularly preferably 1 or 2, and the most preferably 1.

R^{e4} is an aliphatic group having two or more and four or less valences and optionally including an atom other than carbon atom. Examples of the atoms which may be included in R^{e4} include a nitrogen atom, an oxygen atom, a sulfur atom, a fluorine atom, a chlorine atom, a bromine atom, an iodine atom, and the like. A structure of the aliphatic group as R^{e4} may be linear or branched, or may be cyclic, and a structure combining these structures.

Among the compounds represented by the formula (e1), a compound represented by the following formula (e2) is more preferable.

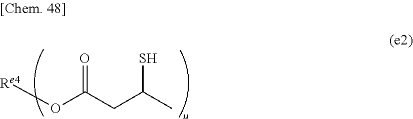

(In the formula (e2), R^{e4} and u are the same as those in the formula (e1).)

Among the compounds represented by the above formula (e2), the following compounds are preferable.

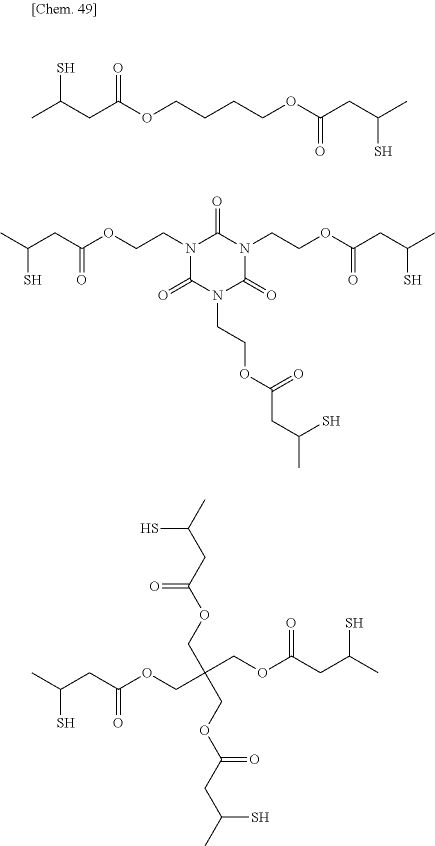

Compounds represented by the following formulae (e3-L1) to (e3-L7) are also preferable examples as the sulfur-containing compound having a mercapto group.

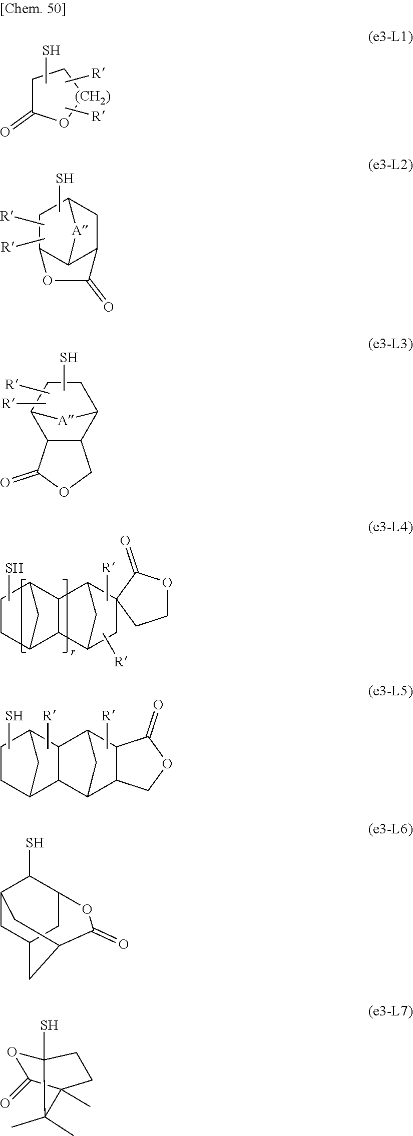

(In the formulae (e3-L1) to (e3-L7), R′, s″, A″, and r are the same as in the formulae (b-L1) to (b-L7) described for the acrylic resin (B3).)

Suitable specific examples of the mercapto compound represented by the above formulae (e3-L1) to (e3-L7) include the following compounds.

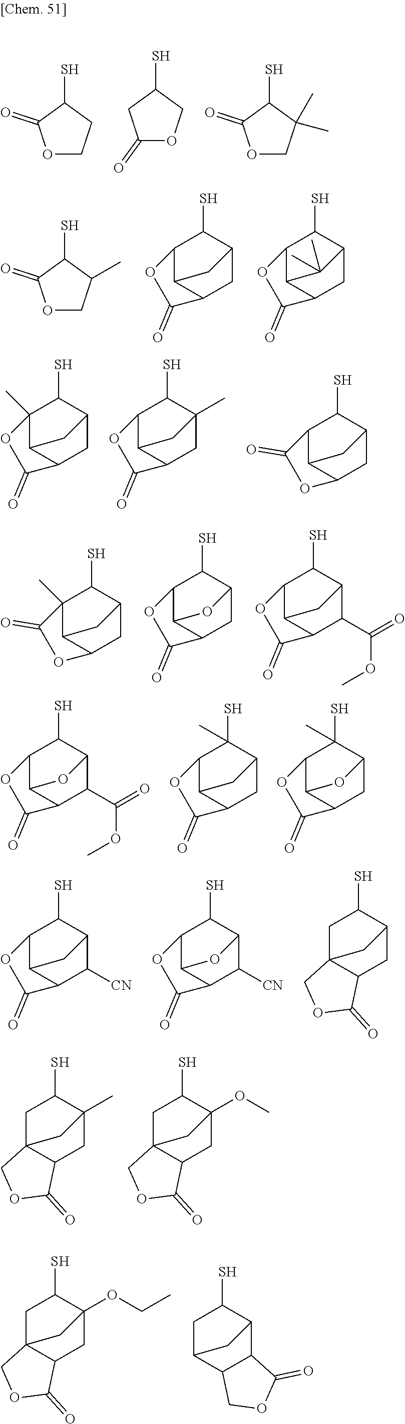

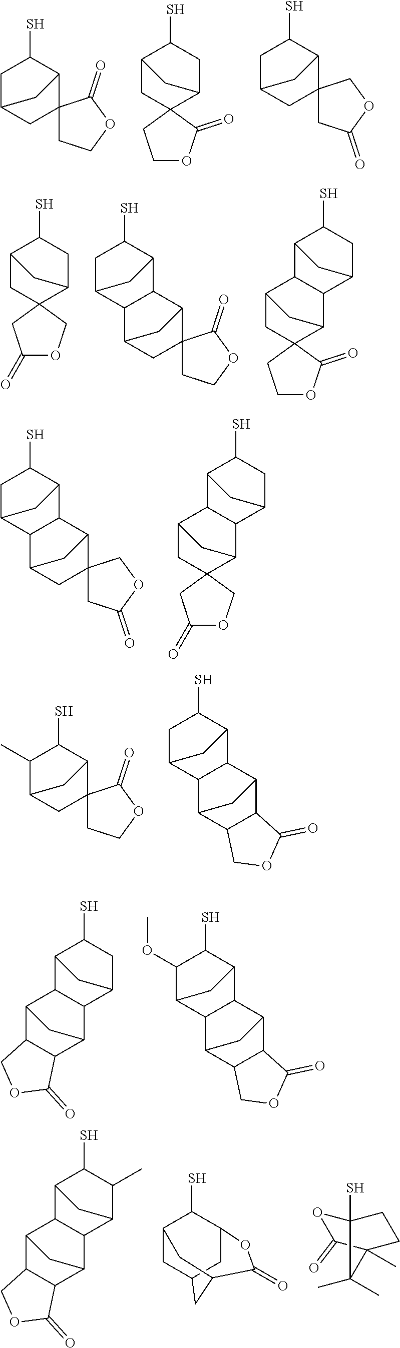

Compounds represented by the following formulae (e3-1) to (e3-4) are also preferable examples as the sulfur-containing compound having a mercapto group.

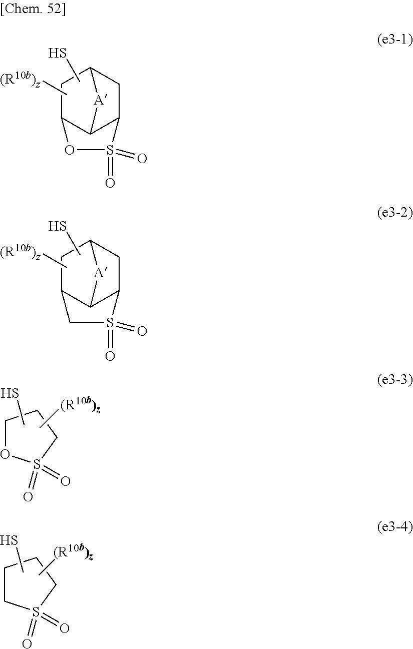

(In the formulae (e3-1) to (e3-4), definitions of abbreviations are the same as mentioned for the formulae (3-1) to (3-4) described for acrylic resin (B3).)

Suitable specific examples of the mercapto compound represented by the above formulae (e3-1) to (e3-4) include the following compounds.

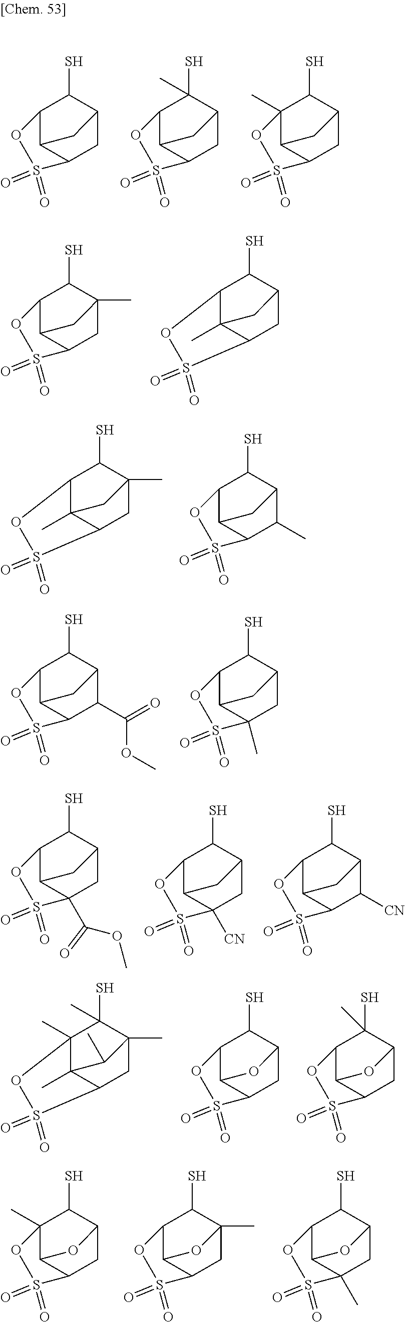

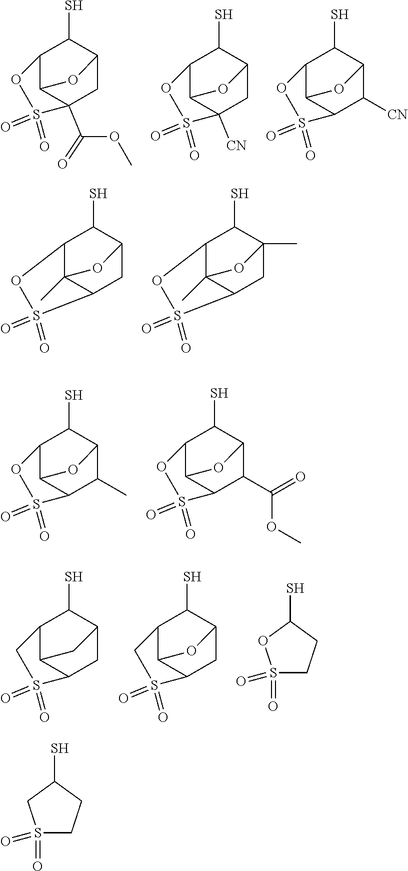

Furthermore, preferable examples of the compound having a mercapto group include compounds represented by the following formula (e4).

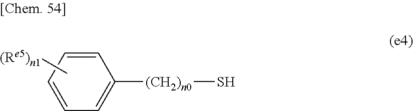

(In the formula (e4), R^{e5} is a group selected from the group consisting of a hydroxyl group, an alkyl group having 1 or more and 4 or less carbon atoms, an alkoxy group having 1 or more and 4 or less carbon atoms, an alkylthio group having 1 or more and 4 or less carbon atoms, a hydroxyalkyl group having 1 or more and 4 or less carbon atoms, a mercapto alkyl group having 1 or more and 4 or less carbon atoms, a halogenated alkyl group having 1 or more and 4 or less carbon atoms, and a halogen atom, n1 is an integer of 0 or more and 3 or less, n0 is an integer of 0 or more and 3 or less, when n1 is 2 or 3, R^{e5} may be the same as or different from each other.)

Specific examples when R^{e5} is an alkyl group which may have a hydroxyl group having 1 or more and 4 or less carbon atoms include a methyl group, an ethyl group, an n-propyl group, an isopropyl group, an n-butyl group, an isobutyl group, a sec-butyl group, and a tert-butyl group. Among these alkyl groups, a methyl group, a hydroxymethyl group, and an ethyl group are preferable.

Specific examples when R^{e5} is an alkoxy group having 1 or more and 4 or less carbon atoms include a methoxy group, an ethoxy group, an n-propyloxy group, an isopropyloxy group, an n-butyloxy group, an isobutyloxy group, a sec-butyloxy group, and a tert-butyloxy group. Among these alkoxy groups, a methoxy group and an ethoxy group are preferable, and a methoxy group is more preferable.

Specific examples when R^{e5} is an alkylthio group having 1 or more and 4 or less carbon atoms include a methylthio group, an ethylthio group, an n-propylthio group, an isopropylthio group, an n-butylthio group, an isobutylthio, a sec-butylthio group, and a tert-butylthio group. Among these alkylthio groups, a methylthio group, and an ethylthio group are preferable, and a methylthio group is more preferable.

Specific examples when R^{e5} is a hydroxyalkyl group having 1 or more and 4 or less carbon atoms include a hydroxymethyl group, a 2-hydroxyethyl group, a 1-hydroxyethyl group, a 3-hydroxy-n-propyl group, and a 4-hydroxy-n-butyl group, and the like. Among these hydroxyalkyl groups, a hydroxymethyl group, a 2-hydroxyethyl group, and a 1-hydroxyethyl group are preferable, and a hydroxymethyl group is more preferable.

Specific examples when R^{e5} is a mercapto alkyl group having 1 or more and 4 or less carbon atoms include a mercapto methyl group, a 2-mercapto ethyl group, a 1-mercapto ethyl group, a 3-mercapto-n-propyl group, a 4-mercapto-n-butyl group, and the like. Among these mercapto alkyl groups, a mercapto methyl group, a 2-mercapto ethyl group, and 1-mercapto ethyl group are preferable, and a mercapto methyl group is more preferable.

When R^{e5} is an alkyl halide group having 1 or more and 4 or less carbon atoms, examples of the halogen atom included in the alkyl halide group include fluorine, chlorine, bromine, iodine, and the like. Specific examples when R^{e5} is an alkyl halide group having 1 or more and 4 or less carbon atoms include a chloromethyl group, a bromomethyl group, an iodomethyl group, a fluoromethyl group, a dichloromethyl group, a dibromomethyl group, a difluoromethyl group, a trichloromethyl group, a tribromomethyl group, a trifluoromethyl group, a 2-chloroethyl group, a 2-bromoethyl group, a 2-fluoroethyl group, a 1,2-dichloroethyl group, a 2,2-difluoroethyl group, a 1-chloro-2-fluoroethyl group, 3-chloro-n-propyl group, a 3-bromon-propyl group, a 3-fluoro-n-propyl group, 4-chloro-n-butyl group, and the like. Among these alkyl halide groups, a chloromethyl group, a bromomethyl group, an iodomethyl group, a fluoromethyl group, a dichloromethyl group, a dibromomethyl group, a difluoromethyl group, a trichloromethyl group, a tribromomethyl group, and a trifluoromethyl group are preferable, and a chloromethyl group, a dichloromethyl group, a trichloromethyl group, and a trifluoromethyl group are more preferable.

Specific examples when R^{e5} is a halogen atom include fluorine, chlorine, bromine, or iodine.

In the formula (e4), n1 is an integer of 0 or more and 3 or less, and 1 is more preferable. When n1 is 2 or 3, a plurality of R^{e5}s may be the same as or different from each other.

In the compound represented by the formula (e4), a substituted position of R^{e5} on a benzene ring is not particularly limited. The substituted position of R^{e5} on a benzene ring is preferably a meta position or a para position with respect to the bond position of —(CH₂)ₙ₀—SH.

The compound represented by the formula (e4) is preferably a compound having at least one group selected from the group consisting of an alkyl group, a hydroxyalkyl group, and a mercapto alkyl group as R^{e5}, and more preferably a compound having one group selected from the group consisting of an alkyl group, a hydroxyalkyl group, and a mercapto alkyl group as R^{e5}. When the compound represented by the formula (e4) has one group selected from the group consisting of an alkyl group, a hydroxyalkyl group, and a mercapto alkyl group as R^{e5}, the substituted position on the benzene ring of the alkyl group, the hydroxyalkyl group, or the mercapto alkyl group is preferably a meta position or a para position with respect to the bond position of —(CH₂)ₙ₀—SH, and more preferably a para position.

In the formula (e4), n0 is an integer of 0 or more and 3 or less. From the viewpoint that preparation or availability of a compound is easy, n0 is preferably 0 or 1, and more preferably 0.

Specific examples of the compound represented by the formula (e4) include p-mercaptophenol, p-thiocresol, m-thiocresol, 4-(methylthio)benzenethiol, 4-methoxybenzenethiol, 3-methoxybenzenethiol, 4-ethoxybenzenethiol, 4-isopropyloxy benzenethiol, 4-tert-butoxybenzenethiol, 3,4-dimethoxy benzenethiol, 3,4,5-trimethoxybenzenethiol, 4-ethylbenzenethiol, 4-isopropyl benzenethiol, 4-n-butylbenzenethiol, 4-tert-butylbenzenethiol, 3-ethylbenzenethiol, 3-isopropyl benzenethiol, 3-n-butylbenzenethiol, 3-tert-butylbenzenethiol, 3,5-dimethyl benzenethiol, 3,4-dimethyl benzenethiol, 3-tert-butyl-4-methylbenzenethiol, 3-tert-4-methylbenzenethiol, 3-tert-butyl-5-methylbenzenethiol, 4-tert-butyl-3-methylbenzenethiol, 4-mercaptobenzyl alcohol, 3-mercaptobenzyl alcohol, 4-(mercaptomethyl)phenol, 3-(mercaptomethyl)phenol, 1,4-di(mercaptomethyl)phenol, 1,3-di(mercaptomethyl)phenol, 4-fluorobenzenethiol, 3-fluorobenzenethiol, 4-chlorobenzenethiol, 3-chlorobenzenethiol, 4-bromobenzenethiol, 4-iodobenzenethiol, 3-bromobenzenethiol, 3,4-dichlorobenzenethiol, 3,5-dichlorobenzenethiol, 3,4-difluorobenzenethiol, 3,5-difluorobenzenethiol, 4-mercaptocatechol, 2,6-di-tert-butyl-4-mercaptophenol, 3,5-di-tert-butyl-4-methoxybenzenethiol, 4-bromo-3-methylbenzenethiol, 4-(trifluoromethyl)benzenethiol, 3-(trifluoromethyl)benzenethiol, 3,5-bis(trifluoromethyl)benzenethiol, 4-methylthiobenzenethiol, 4-ethylthiobenzenethiol, 4-n-butylthiobenzenethiol, and 4-tert-butylthiobenzenethiol, and the like.

Furthermore, examples of the sulfur-containing compound having a mercapto group include a compound including nitrogen-containing aromatic heterocycle substituted with a mercapto group, and a tautomer of a compound including nitrogen-containing aromatic heterocycle substituted with a mercapto group. Preferable specific examples of the nitrogen-containing aromatic heterocycle include imidazole, pyrazole, 1,2,3-triazol, 1,2,4-triazol, oxazole, thiazole, pyridine, pyrimidine, pyridazine, pyrazine, 1,2,3-triazine, 1,2,4-triazine, 1,3,5-triazine, indole, indazole, benzimidazole, benzoxazole, benzothiazole, 1H-benzotriazole, quinoline, isoquinoline, cinnoline, phthalazine, quinazoline, quinoxaline, and 1,8-naphthyridine.

Suitable specific examples of a nitrogen-containing heterocyclic compound suitable as a sulfur-containing compound, and suitable tautomer of the nitrogen-containing heterocyclic compound include the following compounds.

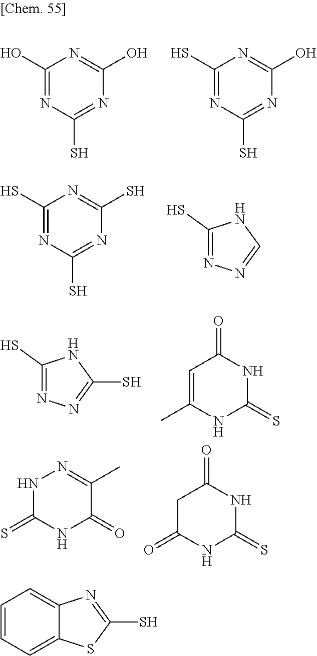

When the photosensitive resin composition includes a sulfur-containing compound (E), the use amount thereof is preferably 0.01 parts by mass or more and 5 parts by mass or less, more preferably 0.02 parts by mass or more and 3 parts by mass or less, and particularly preferably 0.05 parts by mass or more and 2 parts by mass or less with respect to 100 parts by mass that is the total mass of the above resin (B) and the alkali-soluble resin (D) to be described below.

### <Acid Diffusion Controlling Agent (F)>

In order to improve the shape of resist pattern used as a template, the post-exposure delay stability of photosensitive resin film and the like, it is preferable that the photosensitive resin composition further contains an acid diffusion controlling agent (F). The acid diffusion controlling agent (F) is preferably a nitrogen-containing compound (F1), and an organic carboxylic acid, or an oxo acid of phosphorus or a derivative thereof (F2) may be further included as needed.

### [Nitrogen-Containing Compound (F1)]

Examples of the nitrogen-containing compound (F1) include trimethylamine, diethylamine, triethylamine, di-n-propylamine, tri-n-propylamine, tri-n-pentylamine, tribenzylamine, diethanolamine, triethanolamine, n-hexylamine, n-heptyl amine, n-octyl amine, n-nonyl amine, ethylenediamine, N,N,N′,N′-tetramethylethylenediamine, tetramethylenediamine, hexamethylenediamine, 4,4′-diaminodiphenylmethane, 4,4′-diaminodiphenyl ether, 4,4′-diaminobenzophenone, 4,4′-diaminodiphenylamine, formamide, N-methylformamide, N,N-dimethylformamide, acetamide, N-methylacetamide, N,N-dimethylacetamide, propionamide, benzamide, pyrrolidone, N-methylpyrrolidone, methylurea, 1,1-dimethylurea, 1,3-dimethylurea, 1,1,3,3-tetramethylurea, 1,3-diphenylurea, imidazole, benzimidazole, 4-methylimidazole, 8-oxyquinoline, acridine, purine, pyrrolidine, piperidine, 2,4,6-tri(2-pyridyl)-S-triazine, morpholine, 4-methylmorpholine, piperazine, 1,4-dimethylpiperazine, 1,4-diazabicyclo[2.2.2]octane, pyridine, and the like. These may be used alone, or in combinations of two or more thereof.

Furthermore, commercially available hindered amine compounds such as Adeka Stab LA-52, Adeka Stab LA-57, Adeka Stab LA-63P, Adeka Stab LA-68, Adeka Stab LA-72, Adeka Stab LA-77Y, Adeka Stab LA-77G, Adeka Stab LA-81, Adeka Stab LA-82, Adeka Stab LA-87 (all manufactured by ADEKA), 4-hydroxy-1,2,2,6,6-pentamethylpiperidine derivative, and the like, and pyridine whose 2,6-position has been substituted with a substituent a hydrocarbon group such as 2,6-diphenyl pyridine and 2,6-di-tert-butyl pyridine can be used as the nitrogen-containing compound (F1).

The nitrogen-containing compound (F1) may be used in an amount typically in the range of 0 parts by mass or more and 5 parts by mass or less, and particularly preferably in the range of 0 parts by mass or more and 3 parts by mass or less, with respect to 100 parts by mass of total mass of the above resin (B) and the above alkali-soluble resin (D).

### [Organic Carboxylic Acid or Oxo Acid of Phosphorus or Derivative Thereof (F2)]

Among the organic carboxylic acid, or the oxo acid of phosphorus or the derivative thereof (F2), specific preferred examples of the organic carboxylic acid include malonic acid, citric acid, malic acid, succinic acid, benzoic acid, salicylic acid and the like, and salicylic acid is particularly preferred.

Examples of the oxo acid of phosphorus or derivatives thereof include phosphoric acid and derivatives such as esters thereof such as phosphoric acid, phosphoric acid di-n-butyl ester, and phosphoric acid diphenyl ester; phosphonic acid and derivatives such as esters thereof such as phosphonic acid, phosphonic acid dimethyl ester, phosphonic acid di-n-butyl ester, phenylphosphonic acid, phosphonic acid diphenyl ester, and phosphonic acid dibenzyl ester; and phosphinic acid and derivatives such as esters thereof such as phosphinic acid and phenylphosphinic acid; and the like. Among these, phosphonic acid is particularly preferred. These may be used alone, or in combinations of two or more thereof.

The organic carboxylic acid or oxo acid of phosphorus or derivative thereof (F2) may be used in an amount usually in the range of 0 parts by mass or more and 5 parts by mass or less, and particularly preferably in the range of 0 parts by mass and 3 parts by mass or less, with respect to 100 parts by mass of total mass of the above resin (B) and the above alkali-soluble resin (D).

Moreover, in order to form a salt to allow for stabilization, the organic carboxylic acid, or the oxo acid of phosphorous or the derivative thereof (F2) is preferably used in an amount equivalent to that of the above nitrogen-containing compound (F1).

### <Organic Solvent (S)>

It is preferable that the photosensitive resin composition contains an organic solvent (S). There is no particular limitation on the types of the organic solvent (S) as long as the objects of the present invention are not impaired, and an organic solvent appropriately selected from those conventionally used for positive-type photosensitive resin compositions can be used.

Specific examples of the organic solvent (S) include ketones such as acetone, methyl ethyl ketone, cyclohexanone, methyl isoamyl ketone, and 2-heptanone; polyhydric alcohols and derivatives thereof such as glycols such as ethylene glycol, ethylene glycol monoacetate, diethylene glycol, diethylene glycol monoacetate, propylene glycol, propylene glycol monoacetate, propylene glycol monomethyl ether acetate, dipropylene glycol, and a monomethyl ether, a monoethyl ether, a monopropyl ether, a monobutyl ether, and a monophenyl ether of dipropylene glycol monoacetate; cyclic ethers such as dioxane; esters such as ethyl formate, methyl lactate, ethyl lactate, methyl acetate, ethyl acetate, butyl acetate, methyl pyruvate, methyl acetoacetate, ethyl acetoacetate, ethyl pyruvate, ethyl ethoxyacetate, methyl methoxypropionate, ethyl ethoxypropionate, methyl 2-hydroxypropionate, ethyl 2-hydroxypropionate, ethyl 2-hydroxy-2-methylpropionate, methyl 2-hydroxy-3-methylbutanate, 3-methoxybutyl acetate, and 3-methyl-3-methoxybutyl acetate; aromatic hydrocarbons such as toluene and xylene; and the like. These may be used alone, or as a mixture of two or more thereof.

There is no particular limitation on the all content of the organic solvent (S) as long as the objects of the present invention are not impaired. In a case where a photosensitive resin composition is used for a thick-film application such that a photosensitive layer obtained by the spin coating method and the like has a film thickness of 5 μm or more, the organic solvent (S) is preferably used in a range where the solid content concentration of the photosensitive resin composition is 30% by mass or more and 70% by mass or less.

### <Other Components>

The photosensitive resin composition may further contain a polyvinyl resin for improving plasticity. Specific examples of the polyvinyl resin include polyvinyl chloride, polystyrene, polyhydroxystyrene, polyvinyl acetate, polyvinylbenzoic acid, polyvinyl methyl ether, polyvinyl ethyl ether, polyvinyl alcohol, polyvinyl pyrrolidone, polyvinyl phenol, and copolymers thereof, and the like. The polyvinyl resin is preferably polyvinyl methyl ether in view of lower glass transition temperatures.

Further, the photosensitive resin composition may also contain an adhesive auxiliary agent in order to improve the adhesiveness between a resist pattern such as a template formed with the photosensitive resin composition and a substrate.

Also, the photosensitive resin composition may further contain a surfactant for improving coating characteristics, defoaming characteristics, leveling characteristics, and the like. As the surfactant, for example, a fluorine-based surfactant or a silicone-based surfactant is preferably used. Specific examples of the fluorine-based surfactant include commercially available fluorine-based surfactants such as BM-1000 and BM-1100 (both manufactured by B.M-Chemie Co., Ltd.), Megafac F142D, Megafac F172, Megafac F173 and Megafac F183 (all manufactured by Dainippon Ink And Chemicals, Incorporated), Flolade FC-135, Flolade FC-170C, Flolade FC-430 and Flolade FC-431 (all manufactured by Sumitomo 3M Ltd.), Surflon S-112, Surflon S-113, Surflon S-131, Surflon S-141 and Surflon S-145 (all manufactured by Asahi Glass Co., Ltd.), SH-28PA, SH-190, SH-193, SZ-6032 and SF-8428 (all manufactured by Toray Silicone Co., Ltd.) and the like, but not limited thereto. As the silicone-based surfactant, an unmodified silicone-based surfactant, a polyether modified silicone-based surfactant, a polyester modified silicone-based surfactant, an alkyl modified silicone-based surfactant, an aralkyl modified silicone-based surfactant, a reactive silicone-based surfactant, and the like, can be preferably used. As the silicone-based surfactant, commercially available silicone-based surfactant can be used. Specific examples of the commercially available silicone-based surfactant include Paintad M (manufactured by Dow Corning Toray Co., Ltd.), Topica K1000, Topica K2000, and Topica K5000 (all manufactured by Takachiho Industry Co., Ltd.), XL-121 (polyether modified silicone-based surfactant, manufactured by Clariant Co.), BYK-310 (polyester modified silicone-based surfactant, manufactured by BYK), and the like.

Additionally, in order to finely adjust the solubility in a developing solution, the photosensitive resin composition may further contain an acid, an acid anhydride, or a solvent having a high boiling point.

Specific examples of the acid and acid anhydride include monocarboxylic acids such as acetic acid, propionic acid, n-butyric acid, isobutyric acid, n-valeric acid, isovaleric acid, benzoic acid, and cinnamic acid; hydroxymonocarboxylic acids such as lactic acid, 2-hydroxybutyric acid, 3-hydroxybutyric acid, salicylic acid, m-hydroxybenzoic acid, p-hydroxybenzoic acid, 2-hydroxycinnamic acid, 3-hydroxycinnamic acid, 4-hydroxycinnamic acid, 5-hydroxyisophthalic acid, and syringic acid; polyvalent carboxylic acids such as oxalic acid, succinic acid, glutaric acid, adipic acid, maleic acid, itaconic acid, hexahydrophthalic acid, phthalic acid, isophthalic acid, terephthalic acid, 1,2-cyclohexanedicarboxylic acid, 1,2,4-cyclohexanetricarboxylic acid, butanetetracarboxylic acid, trimellitic acid, pyromellitic acid, cyclopentanetetracarboxylic acid, butanetetracarboxylic acid, and 1,2,5,8-naphthalenetetracarboxylic acid; acid anhydrides such as itaconic anhydride, succinic anhydride, citraconic anhydride, dodecenylsuccinic anhydride, tricarbanilic anhydride, maleic anhydride, hexahydrophthalic anhydride, methyltetrahydrophthalic anhydride, Himic anhydride, 1,2,3,4-butanetetracarboxylic anhydride, cyclopentanetetracarboxylic dianhydride, phthalic anhydride, pyromellitic anhydride, trimellitic anhydride, benzophenonetetracarboxylic anhydride, ethylene glycol bis anhydrous trimellitate, and glycerin tris anhydrous trimellitate; and the like.

Furthermore, specific examples of the solvent having a high boiling point include N-methylformamide, N,N-dimethylformamide, N-methylformanilide, N-methylacetamide, N,N-dimethlyacetamide, N-methylpyrrolidone, dimethyl sulfoxide, benzyl ethyl ether, dihexyl ether, acetonyl acetone, isophorone, caproic acid, caprylic acid, 1-octanol, 1-nonanol, benzyl alcohol, benzyl acetate, ethyl benzoate, diethyl oxalate, diethyl maleate, γ-butyrolactone, ethylene carbonate, propylene carbonate, phenyl cellosolve acetate, and the like.

Moreover, the photosensitive resin composition may further contain a sensitizer for improving the sensitivity.

### <Method of Preparing Chemically Amplified Positive-Type Photosensitive Resin Composition>

The chemically amplified positive-type photosensitive resin composition is prepared by mixing and stirring the above components by the common method. Machines which can be used for mixing and stirring the above components include dissolvers, homogenizers, 3-roll mills and the like. After uniformly mixing the above components, the resulting mixture may be filtered through a mesh, a membrane filter and the like.

### <<Photosensitive Dry Film>>

The photosensitive dry film includes a substrate film, and a photosensitive layer formed on the surface of the substrate film, the photosensitive layer being formed of the photosensitive resin composition.

As the substrate film, a film having optical transparency is preferable. Specifically, a polyethylene terephthalate (PET) film, a polypropylene (PP) film, a polyethylene (PE) film, and the like. In view of excellent balance between the optical transparency and the breaking strength, a polyethylene terephthalate (PET) film is preferable.

The aforementioned photosensitive resin composition is applied on the substrate film to form a photosensitive layer, and thereby a photosensitive dry film is manufactured. When the photosensitive layer is formed on the substrate film, a photosensitive resin composition is applied and dried on the substrate film using an applicator, a bar coater, a wire bar coater, a roller coater, a curtain flow coater, and the like, so that a film thickness after drying is preferably 0.5 μm or more and 300 μm or less, more preferably 1 μm or more and 300 μm or less, and particularly preferably 3 μm or more and 100 μm or less.

The photosensitive dry film may have a protective film on the photosensitive layer. Examples of the protective film include a polyethylene terephthalate (PET) film, a polypropylene (PP) film, a polyethylene (PE) film, and the like.

### <<Method of Producing Patterned Resist Film>>

There is no particular limitation on a method of forming a patterned resist film on a substrate using the photosensitive resin composition described above. Such a patterned resist film is used as a template for forming a plated article. Such a patterned resist film is suitably used as a template for forming a plated article, an etching mask for processing a substrate by etching, or the like. A suitable method includes a manufacturing method of a patterned resist film, the method including: laminating a photosensitive layer on a substrate, the layer being formed from a photosensitive resin composition; exposing the photosensitive layer through irradiation with an active ray or radiation in a position-selective manner; and developing the exposed photosensitive layer. A method of manufacturing a substrate with a template for forming a plated article is the same method as the method of manufacturing a patterned resist film except that the method includes laminating a photosensitive layer on a metal surface of the substrate having a metal surface, and a template for forming a plated article is produced by developing in the developing step.

The substrate on which the photosensitive layer is laminated is not particularly limited, and a conventionally known substrate can be used, and examples thereof include a substrate for an electronic component, a substrate having a predetermined wiring pattern formed thereon, and the like. As the substrate, a silicon substrate, a glass substrate, or the like can also be used. When manufacturing a substrate with a template for forming a plated article, a substrate having a metal surface is used as the substrate. As metal species constituting the metal surface, copper, gold and aluminum are preferred, and copper is more preferred.

The photosensitive layer is laminated on the substrate, for example, as follows. That is, a liquid photosensitive resin composition is coated onto a substrate, and the coating is heated to remove the solvent and thus to form a photosensitive layer having a desired thickness. The thickness of the photosensitive layer is not particularly limited as long as it is possible to form a resist pattern serving as a template which has a desired thickness. The thickness of the photosensitive layer is not particularly limited, but is preferably 0.5 μm or more, more preferably 0.5 μm or more and 300 μm or less, and particularly preferably 1 μm or more and 150 μm or less, and most preferably 3 μm or more and 100 μm or less.

As a method of applying a photosensitive resin composition onto a substrate, methods such as the spin coating method, the slit coat method, the roll coat method, the screen printing method and the applicator method can be employed. Pre-baking is preferably performed on a photosensitive layer. The conditions of pre-baking may differ depending on the components in a photosensitive resin composition, the blending ratio, the thickness of a coating film and the like. They are usually about 2 minutes or more and 120 minutes or less at 70° C. or more and 200° C. or less, and preferably 80° C. or more and 150° C. or less.

The photosensitive layer formed as described above is selectively irradiated (exposed) with an active ray or radiation, for example, an ultraviolet radiation or visible light with a wavelength of 300 nm or more and 500 nm or less through a mask having a predetermined pattern.

Low pressure mercury lamps, high pressure mercury lamps, super high pressure mercury lamps, metal halide lamps, argon gas lasers, etc. can be used for the light source of the radiation. The radiation may include microwaves, infrared rays, visible lights, ultraviolet rays, X-rays, γ-rays, electron beams, proton beams, neutron beams, ion beams, etc. The irradiation dose of the radiation may vary depending on the constituent of the photosensitive resin composition, the film thickness of the photosensitive layer, and the like. For example, when an ultra-high-pressure mercury lamp is used, the dose may be 100 mJ/cm² or more and 10,000 mJ/cm² or less. The radiation includes a light ray to activate the acid generator (A) in order to generate an acid.

After the exposure, the diffusion of acid is promoted by heating the photosensitive layer using a known method to change the alkali solubility of the photosensitive layer at an exposed portion in the photosensitive resin film.

Subsequently, the exposed photosensitive layer is developed in accordance with a conventionally known method, and an unnecessary portion is dissolved and removed to form a predetermined resist pattern or a template for forming a plated article. At this time, as the developing solution, an alkaline aqueous solution is used.

As the developing solution, an aqueous solution of an alkali such as, for example, sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, dimethylethanolamine, triethanolamine, tetramethylammonium hydroxide, tetraethylammonium hydroxide, pyrrole, piperidine, 1,8-diazabicyclo[5,4,0]-7-undecene or 1,5-diazabicyclo[4,3,0]-5-nonane can be used. Also, an aqueous solution obtained by adding an adequate amount of a water-soluble organic solvent such as methanol or ethanol, or a surfactant to the above aqueous solution of the alkali can be used as the developing solution.

The developing time may vary depending on the constituent of the photosensitive resin composition, the film thickness of the photosensitive layer, and the like. Usually, the developing time is 1 minute or more and 30 minutes or less. The method of the development may be any one of a liquid-filling method, a dipping method, a paddle method, a spray developing method, and the like.

After development, it is washed with running water for 30 seconds or more and 90 seconds or less, and then dried with an air gun, an oven, and the like. In this manner, a resist pattern, which has been patterned in a desired shape, is formed on a surface of a substrate. Also, in this manner, it is possible to manufacture a substrate with a template having a resist pattern serving as a template, on a metal surface of a substrate having a metal surface. Since the photosensitive resin composition can form a resist pattern excellent in mask linearity, a resist pattern having a desired dimension can be formed.

A conductor such as a metal may be embedded, by plating, into a nonresist portion (a portion removed with a developing solution) in the template in the substrate with a template formed by the above method to form a plated article, for example, like a contacting terminal such as a bump and a metal post, or Cu redistribution line. Note here that there is no particular limitation on the method of plate processing, and various conventionally known methods can be used. As a plating liquid, in particular, a solder plating liquid, a copper plating liquid, a gold plating liquid, and a nickel plating liquid are suitably used. Finally, the remaining template is removed with a stripping liquid and the like in accordance with a conventional method.

When the plated article is manufactured, it may be preferable that an exposed metal surface in a non-patterned portion of a resist pattern serving as a template for forming plated article is subjected to asking treatment. Specific examples include case where a pattern formed of a photosensitive resin composition including a sulfur-containing compound (E) is used as a template to form a plated article. In this case, adhesiveness of the plated article to a metal surface may be easily damaged. This problem is remarkable in a case where sulfur-containing compound (E) represented by the above-mentioned formula (e1), and the sulfur-containing compound (E) represented by the formula (e4). However, the above-mentioned ashing treatment is carried out, even when a pattern formed using a photosensitive resin composition including a sulfur-containing compound (E) is used as a template, a plated article favorably adhering to the metal surface is easily formed. Note here that in a case where a compound including a nitrogen-containing aromatic heterocycle substituted with a mercapto group is used as a sulfur-containing compound (E), the problem of adhesiveness of a plated article hardly occurs or occurs slightly. Therefore, in a case where a compound including a nitrogen-containing aromatic heterocycle substituted with a mercapto group is used as a sulfur-containing compound (E), a plated article having excellent adhesiveness with respect to the metal surface is easily formed without carrying out ashing treatment.

The ashing treatment is not particularly limited as long as long as it does not damage a resist pattern serving as a template for forming the plated article to such an extent that the plated article having a desired shape cannot be formed. Preferable ashing treatment methods include a method using oxygen plasma. For ashing with respect to the metal surface of the substrate using oxygen plasma, an oxygen plasma is generated using a known oxygen plasma generator, and the metal surface on the substrate is irradiated with the oxygen plasma.

Various gases which have conventionally been used for plasma treatment together with oxygen can be mixed into gas to be used for generating oxygen plasma within a range where the objects of the present invention are not impaired. Examples of such gas include nitrogen gas, hydrogen gas, CF₄ gas, and the like. Conditions of asking using oxygen plasma are not particularly limited within a range where the objects of the present invention are not impaired, but treatment time is, for example, in a range of 10 seconds or more and 20 minutes or less, preferably in a range of 20 seconds or more and 18 minutes or less, and more preferably in a range of 30 seconds or more and 15 minutes or less. By setting the treatment time by oxygen plasma to the above range, an effect of improving the adhesiveness of the plated article can be easily achieved without changing a shape of the resist pattern.

The compound represented by the formula (A1) is a novel compound and may be used as a photoacid generator as described above.

### <<Method of Manufacturing N-Organosulfonyloxy Compound>>

A method of manufacturing a N-organosulfonyloxy compound which can be applied to the production of the compound represented by the above formula (A1) will be described. N-organosulfonyloxy compounds having a structure in which an organosulfonyloxy group is bonded to a nitrogen atom of a nitrogen-containing compound, such as an organosulfonate ester of oxime or N-hydroxynaphthalimide, are known as photoacid generators which can be incorporated into, for example, a chemically amplified photosensitive resin composition (Japanese Unexamined Patent Application, Publication No. 2010-159243). As a method for synthesizing such an N-organosulfonyloxy compound, a method is known, for example, in which an organic sulfonyl fluoride compound is reacted with an N-hydroxy compound, which has a structure in which a hydroxy group is bonded to a nitrogen atom of a nitrogen-containing compound, in the presence of a base (see, for example, Japanese Unexamined Patent Application, Publication No. 2010-159243). However, in this method, there is a problem that the reaction does not easily proceed and a target N-organosulfonyloxy compound cannot be obtained or can be obtained only in a poor yield, even if obtained. Therefore, there is a need for an efficient method of manufacturing N-organosulfonyloxy compounds.

Such a problem can be solved by the following method of manufacturing a N-organosulfonyloxy compound. The following method of manufacturing a N-organosulfonyloxy compound can be applied to the production of a compound represented by the formula (A1). The method of manufacturing a N-organosulfonyloxy compound includes reacting a N-hydroxy compound (A′) with a sulfonyl fluoride compound (B′) in the presence of a basic compound (D′). Furthermore, the method of manufacturing a N-organosulfonyloxy compound is characterized in that a silylating agent (C′) is present in the system, while the N-hydroxy compound (A′) and the sulfonyl fluoride compound (B′) are reacted. The sulfonyl fluoride compound (B′) is represented by the following formula (bi1), and the silylating agent (C′) is a compound which can convert a hydroxy group on a nitrogen atom which the N-hydroxy compound (A′) has into a silyloxy group represented by the following formula (ac1).

The N-hydroxy compound (A′) has a structure in which a hydroxy group is bonded to a nitrogen atom of a nitrogen-containing compound. When the N-hydroxy compound contains a plurality of nitrogen atoms in one molecule, hydroxy groups may be bonded to two or more nitrogen atoms of the plurality of nitrogen atoms. In the N-hydroxy compound (A′), it is preferable that one or two carbonyl groups are present at a position(s) adjacent to the nitrogen atom to which the hydroxy group is bonded. Examples of the N-hydroxy compound (A′) in which a carbonyl group is present at a position adjacent to a nitrogen atom to which the hydroxy group is bonded include N-hydroxyamide compounds represented by R^{a0}—NOH—CO—R^{a0}, N-hydroxyurea compounds represented by R^{a0}—NOH—CO—N(R^{a0})₂, and N-hydroxycarbamate compounds represented by R^{a0}—NOH—CO—O—R^{a0}. Here, R^{a0}s are each independently a hydrogen atom or an organic group. As the organic group as R^{a0}, a hydrocarbon group having 1 or more and 20 or less carbon atoms is preferred, a hydrocarbon group having 1 or more and 10 or less carbon atoms is more preferred, and a hydrocarbon group having 1 or more and 6 or less carbon atoms is most preferred. The hydrocarbon group as R^{a0} may be linear, branched, cyclic or a combined structure thereof. Preferred examples of the hydrocarbon group as R^{a0} include: a methyl group, an ethyl group, a n-propyl group, an isopropyl group, a n-butyl group, an isobutyl group, a sec-butyl group, a tert-butyl group, and a phenyl group. Preferred examples of the N-hydroxy compound (A′) in which a carbonyl group is present at a position adjacent to a nitrogen atom to which a hydroxy group is bonded include N-hydroxy acetamide, N-hydroxy propionamide, N-hydroxy benzamide, and hydroxy carbamide (N-hydroxyurea). As the N-hydroxy compound (A′) in which two carbonyl groups are present at the positions adjacent to the nitrogen atom to which a hydroxy group is bonded, N-hydroxyimide compounds represented by R^{a0}—CO—OH—CO—R^{a0}, N-hydroxysuccinimide compounds which may have one or more substituents on the ring, and N-hydroxymaleimide compounds which may have one or more substituents on the ring are preferred. N-hydroxy aromatic dicarboxylic acid imide compounds which may have one or more substituents on the aromatic ring, such as N-hydroxyphthalimide compounds which may have one or more substituents on the benzene ring and N-hydroxynaphthalimide compounds which may have one or more substituents on the naphthalene ring are also preferred. R^{a0} is as previously described. Preferred examples of the N-hydroxy compound (A′) in which two carbonyl groups are present at the positions adjacent to the nitrogen atom to which a hydroxy group is bonded include N-hydroxysuccinimide which may have one or more substituents on the ring, N-hydroxymaleimide which may have one or more substituents on the ring, N-hydroxyphthalimide which may have one or more substituents on the benzene ring, and N-hydroxynaphthalimide which may have one or more substituents on the naphthalene ring. In the N-hydroxy compound in which two carbonyl groups are present at the positions adjacent to a nitrogen atom to which a hydroxy group is bonded, examples of the substituent which may be present on a ring such as a benzene ring or a naphthalene ring include a halogen atom, a nitro group, a cyano group, and organic groups. The organic group may contain a hetero atom such as N, O, P, S, Se, etc. Examples of the organic group as the substituent include the same groups as groups as R^{a1} and R^{a2} in formula (ai1-1) to be described below, other than a hydrogen atom.

Among the above-mentioned N-hydroxy compounds (A′), a N-hydroxynaphthalimide compound which may have one or more substituents on the naphthalene ring is preferred from the viewpoint of usefulness of the N-organosulfonyloxy compound to be generated, as a photoacid generator. Suitable examples of the N-hydroxynaphthalimide compound which may have one or more substituents on the naphthalene ring include a compound represented by the following formula (ai1-1):

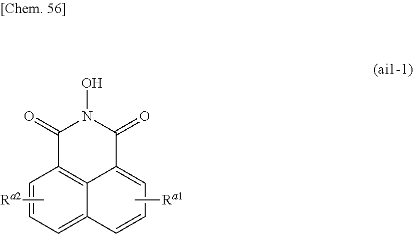

(in formula (ai1-1), R^{a1} and R^{a2} are each independently a hydrogen atom, an optionally substituted aliphatic hydrocarbon group having 1 or more and 20 or less carbon atoms, an optionally substituted aromatic group having 5 or more and 20 or less ring constituting atoms, or a group represented by —R^{a3}—Rₐ₄ when the aliphatic hydrocarbon group as R^{a1} and R^{a2} contains 1 or more methylene groups, at least a part of the methylene groups may be replaced with a group selected from the group consisting of —O—, —S—, —CO—, —CO—O—, —SO—, —SO₂—, and —NR^{a5}—, R^{a5} is a hydrogen atom or a hydrocarbon group having 1 or more and 6 or less carbon atoms, R^{a3} is a methylene group, —O—, —CO—, —CO—O—, —SO—, —SO₂—, or —NR^{a6}—, R^{a6} is a hydrogen atom or a hydrocarbon group having 1 or more and 6 or less carbon atoms, and R^{a4} is a heteroarylalkyl group containing an optionally substituted aromatic group having 5 or more and 20 or less ring constituting atoms, a perfluoroalkyl group having 1 or more and 6 or less carbon atoms, an optionally substituted aralkyl group having 7 or more and 20 or less carbon atoms, or an optionally substituted aromatic heterocyclic group having 5 or more and 20 or less ring constituting atoms.)

In the compound represented by formula (ai1-1), the direction of —CO—O— is not particularly limited.

In formula (ai1-1), the aliphatic hydrocarbon group having 1 or more and 20 or less carbon atoms as R^{a1} and R^{a2} may be linear, branched, cyclic, or a combination of these structures. As the aliphatic hydrocarbon group, an alkyl group is preferred. Suitable examples of the alkyl group include a methyl group, an ethyl group, a n-propyl group, an isopropyl group, a n-butyl group, an isobutyl group, a sec-butyl group, a tert-butyl group, a n-pentyl group, a n-hexyl group, a n-heptyl group, a n-octyl group, a 2-ethylhexyl group, a n-nonyl group, and a n-decyl group. Examples of the substituent which the aliphatic hydrocarbon group having 1 or more and 20 or less carbon atoms as R^{a1} and R^{a2} may have include a hydroxy group, a mercapto group, an amino group, a halogen atom, an oxygen atom, a nitro group, a cyano group, etc. The number of substituents is any number. Examples of the aliphatic hydrocarbon group having 1 or more and 20 or less carbon atoms having a substituent as R^{a1} and R^{a2} include a perfluoroalkyl group having 1 or more and 6 or less carbon atoms. Examples include CF₃—, CF₃CF₂—, (CF₃)₂CF—, CF₃CF₂CF₂—, CF₃CF₂CF₂CF₂—, (CF₃)₂CFCF₂—, CF₃CF₂ (CF₃) CF—, and (CF₃)₃C—.

In formula (ai1-1), the optionally substituted aromatic group having 5 or more and 20 or less ring constituting atoms as R^{a1} and R^{a2} may be an aromatic hydrocarbon group or an aromatic heterocyclic group. Examples of the aromatic group include aryl groups such as a phenyl group and a naphthyl group, and heteroaryl groups such as a furyl group and a thienyl group. The substituents which may be possessed by the aromatic group having 5 or more and 20 or less ring constituting atoms are the same as the substituents which may be possessed by the aliphatic hydrocarbon group having 1 or more and 20 or less carbon atoms as R^{a1} and R^{a2}.

In formula (ai1-1), R^{a4} to R^{a6} are the same as R^{a4} to R^{a6} in the formula (A1), respectively.

As the N-hydroxy compound (A′), oximes may be used.

A method of obtaining the N-hydroxy compound (A′), such as a method of synthesizing the N-hydroxy compound (A′) and the like, is as described above.

The sulfonyl fluoride compound (B′) used in the method of manufacturing a N-organosulfonyloxy compound is a compound represented by the following formula (bi1):

R^{b11}—SO₂—F (bi1)

(in formula (bi1), R^{b11} is an organic group).

In formula (bi1), the organic group as R^{b11} is not particularly limited, and may be selected depending on the target N-organosulfonyloxy compound. As an example, the organic group as R^{b11} is the same group as the group as R^{a1} and R^{a2} in the aforementioned formula (ai1-1), other than a hydrogen atom.

Examples of the compound represented by the above formula (bi1) include a compound represented by the following formula (bi1-1):

R^{bi2}—CQ¹Q²-SO₂—F (bi1-1)

(in formula (bi1-1), Q¹ and Q² are each independently a fluorine atom or a perfluoroalkyl group having 1 or more and 6 or less carbon atoms, and R^{bi2} is an organic group).

In formula (bi1-1), the perfluoroalkyl group having 1 or more and 6 or less carbon atoms as Q¹ and Q² is the same as the perfluoroalkyl group having 1 or more and 6 or less carbon atoms described as R^{a1} and R^{a2} in formula (ai1-1).

In formula (bi1-1), the organic group as R^{bi2} is not particularly limited, and may be selected depending on the target N-organosulfonyloxy compound. As an example, the organic group as R^{bi2} is the same group as the group as R^{a1} and R^{a2} in the aforementioned formula (ai1-1), other than a hydrogen atom.

As the compound represented by the above formula (bi1), a compound represented by the following formula (bi1-2) is preferred.

R^{bi3}-Lⁱ-CQ¹Q²-SO₂—F (bi1-2)

(In formula (bi1-2), Q¹ and Q² are the same as those in formula (bi1-1), Lⁱ is —CO—O— or —O—, R^{bi3} is a hydrocarbon group having 1 or more and 30 or less carbon atoms, when the hydrocarbon group as R^{bi3} contains 1 or more methylene groups, at least a part of the methylene groups may be replaced with a group selected from the group consisting of —O—, —S—, —CO—, —CO—O—, —SO—, —SO₂—, —CR^{b4}R^{b5}—, and —NR^{b6}—, when the hydrocarbon group as R^{bi3} contains a hydrocarbon ring, at least one of the carbon atoms constituting the hydrocarbon ring may be replaced with a heteroatom selected from the group consisting of N, O, P, S, and Se or an atomic group containing the heteroatom, R^{b4} and R^{b5} are each independently a hydrogen atom, or a halogen atom, and at least one of R^{b4} and R^{b5} is a halogen atom, and R^{b6} is a hydrogen atom or a hydrocarbon group having 1 or more and 6 or less carbon atoms).

In the compound represented by formula (bi1-2), the direction of —CO—O— is not particularly limited.

In formula (bi1-2), the hydrocarbon group having 1 or more and 30 or less carbon atoms as R^{bi3} may be an aliphatic hydrocarbon group or an aromatic hydrocarbon group or a combination thereof. The aliphatic hydrocarbon group may be linear, branched, cyclic or a combination of these structures. Examples of the aliphatic hydrocarbon group include a chain-like aliphatic hydrocarbon group such as a methyl group, an ethyl group, a n-propyl group, an isopropyl group, a n-butyl group, an isobutyl group, a sec-butyl group, a tert-butyl group, a n-pentyl group, and a n-hexyl group, and a cyclic aliphatic hydrocarbon group (hydrocarbon ring) such as a cyclopropyl group, a cyclobutyl group, a cyclopentyl group, a cyclohexyl group, an adamantyl group, and a norbornyl group. Examples of the aromatic hydrocarbon group include a phenyl group and a naphthyl group. Examples of the group in which an aliphatic hydrocarbon group and an aromatic hydrocarbon group are combined include a benzyl group, a phenethyl group, and a furyl methyl group. When the hydrocarbon group as R^{bi3} contains a hydrocarbon ring, examples of the atomic group containing a hetero atom which replaces at least one of the carbon atoms constituting the hydrocarbon ring include —CO—, —CO—O—, —SO—, and —SO₂—, —SO₂—O—, —P(═O)—(OR^{b7})₃. R^{b7} is a hydrocarbon group having 1 or more and 6 or less carbon atoms, and is the same as the hydrocarbon group having 1 or more and 6 or less carbon atoms described for R^{a5}.

In the formula (bi1-2), examples of halogen atoms as R^{b4} and R^{b5} include a chlorine atom, a fluorine atom, a bromine atom, and an iodine atom.

In formula (bi1-2), the hydrocarbon group having 1 or more and 6 or less carbon atoms as R^{b6} is the same as the hydrocarbon group having 1 or more and 6 or less carbon atoms described for R^{a5} in formula (ai1-1).

The sulfonyl fluoride compound (B′) can be synthesized by a conventional method. For example, a compound represented by formula (bi1-2) in which Lⁱ is —CO—O— and Q¹ and Q² are fluorine atoms can be synthesized by the reaction represented by the following formula. Further, a commercially available product may be used as the sulfonyl fluoride compound (B′).

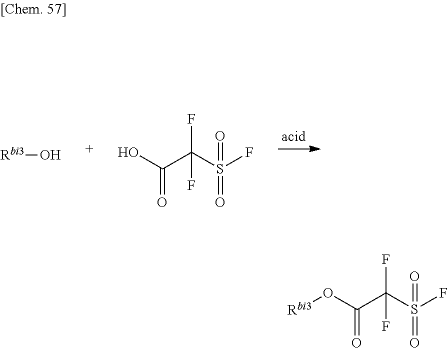

The silylating agent (C′) used in the method of manufacturing a N-organosulfonyloxy compound is a compound capable of converting a hydroxy group on a nitrogen atom possessed by the N-hydroxy compound (A′) into a silyloxy group represented by the following formula (ac1):

—O—Si(R^{c1})₃ (ac1)

(in formula (ac1), each R^{c1} is independently a hydrocarbon group having 1 or more and 10 or less carbon atoms).

In formula (ac1), a hydrocarbon group having 1 or more and 10 or less carbon atoms as R^{c1} may be an aliphatic hydrocarbon group, an aromatic hydrocarbon group, or a combination thereof. The aliphatic hydrocarbon group may be linear, branched, cyclic or a combination of these structures. Aliphatic hydrocarbon groups include alkyl groups such as a methyl group, an ethyl group, a n-propyl group, an isopropyl group, a n-butyl group, an isobutyl group, a sec-butyl group, a tert-butyl group, a n-pentyl group, a n-hexyl group, a n-heptyl group, a n-octyl group, a 2-ethylhexyl group, a n-nonyl group, and a n-decyl group, etc. Examples of the aromatic hydrocarbon group include a phenyl group and a naphthyl group.

Examples of the silylating agent (C′) include a compound represented by the following formula (c1):

X—Si(R^{c1})₃ (c1)

(in formula (c1), R^{c1} is the same as R^{c1} in formula (ac1), and X is a halogen atom).

In formula (c1), examples of halogen atoms as X include a chlorine atom, a fluorine atom, a bromine atom, and an iodine atom.

Examples of the silylating agent (C′) are the same as the examples of the silylating agent (C′) described above.

The basic compound (D′) used in the method of manufacturing a N-organosulfonyloxy compound may be an organic base or an inorganic base. Examples of the organic base include a nitrogen-containing basic compound, and examples thereof are the same as examples of the basic compound (D′) described above. Examples of the inorganic base include metal hydroxides, metal hydrogen carbonates, and metal bicarbonates. Examples of the inorganic base are the same as those of the basic compound (D′) described above.

In the method of manufacturing a N-organosulfonyloxy compound, such a N-hydroxy compound (A′) and a sulfonyl fluoride compound (B′) are reacted in the presence of a silylating agent (C′) and a basic compound (D′). As described above, in the reaction of the N-hydroxy compound (A′) and the sulfonyl fluoride compound (B′) in the presence of the basic compound (D′), presence of the silylating agent (C′) enables efficient production of the N-organosulfonyloxy compound, as shown in the Examples to be described below. For example, the N-organosulfonyloxy compound can be obtained in a yield of 65% or more with respect to the N-hydroxy compound (A′) and the sulfonyl fluoride compound (B′) as the raw materials.

By the method of manufacturing a N-organosulfonyloxy compound, a N-organosulfonyloxy compound is obtained, which has a structure in which a group obtained by removing a hydrogen atom from a hydroxy group bonded to the nitrogen atom of the N-hydroxy compound (A′) and R^{b11}—SO₂— derived from the sulfonyl fluoride compound (B′) are bonded. Examples of the obtained N-organosulfonyloxy compound include a compound represented by the following formula:

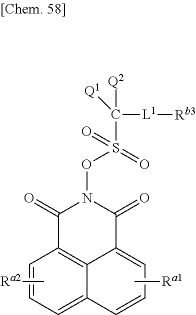

(in which R^{a1} and R^{a2} are the same as R^{a1} and R^{a2}, respectively, in formula (ai1-1) and Q², Lⁱ, and R^{bi3} are the same as Q¹, Q², Lⁱ and R^{bi3}, respectively, in formula (bi1-2).)

In the method of manufacturing a N-organosulfonyloxy compound, when the N-hydroxy compound (A′) and the sulfonyl fluoride compound (B′) are reacted in the presence of the basic compound (D′), it is sufficient for the silylating agent (C′) to exist in the reaction system. The N-hydroxy compound (A′), the sulfonyl fluoride compound (B′), the silylating agent (C′) and the basic compound (D′) may be mixed simultaneously or after the N-hydroxy compound (A′) and the silylating agent (C′) are partially or completely reacted, the sulfonyl fluoride (B′) and the basic compound (D′) may be added.

When the N-hydroxy compound (A′) and sulfonyl fluoride compound (B′) are reacted in the presence of the silylating agent (C′) and the basic compound (D′), as described above, the N-hydroxy compound (A′) is silylated by the silylating agent (C′) and the hydroxy group on the nitrogen atom is converted into a silyloxy group represented by the above formula (ac1) (Step 1: silylation step). Then, the silylated product of the N-hydroxy compound (A′) generated in the silylation step is condensed with the sulfonyl fluoride compound (B′) with which the basic compound (D′) has reacted (Step 2: condensation step). This gives the N-organosulfonyloxy compound.

As an example of the method of manufacturing a N-organosulfonyloxy compound, a reaction formula is shown below, in which a compound represented by the above formula (ai1-1) is used as the N-hydroxy compound (A′), a compound represented by the above formula (bi1-2), in which Lⁱ is —CO—O— and Q¹ and Q² are fluorine atoms, is used as the sulfonyl fluoride compound (B′), trimethylsilyl chloride is used as the silylating agent (C′), and triethylamine is used as the basic compound (D′). Note that the reaction mechanism shown below is not an analytically confirmed mechanism, but a reaction mechanism estimated from raw materials and behaviors thereof during the reaction.

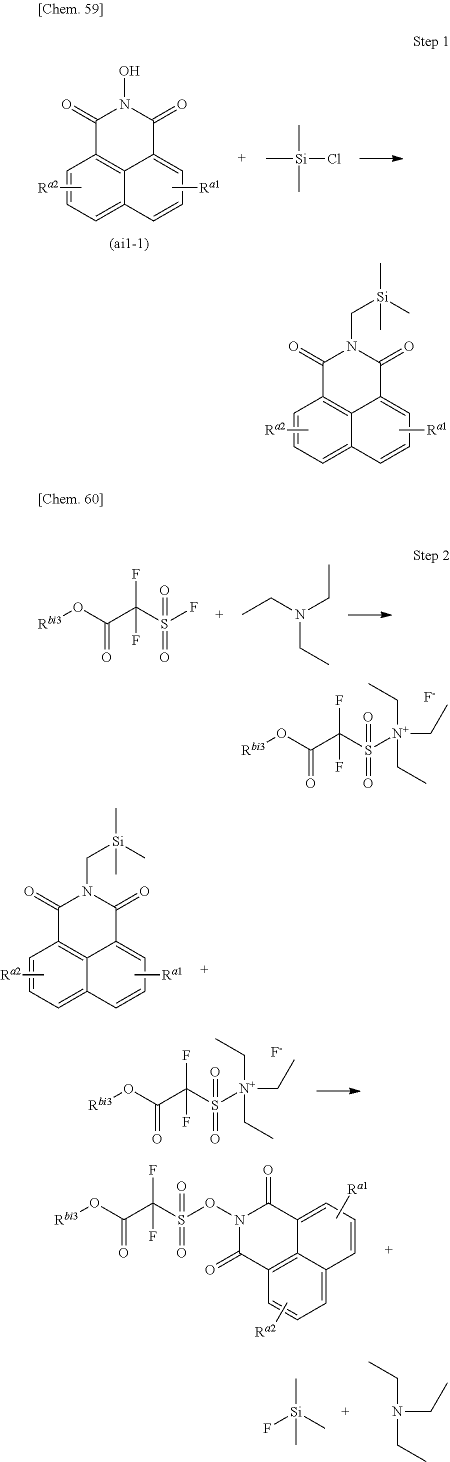

Examples of an organic solvent which can be adopted for the reaction include: esters, such as ethyl acetate, butyl acetate, cellosolve acetate, and the like, ketones such as methyl ethyl ketone, isobutyl ketone, and methyl isobutyl ketone, etc., esters such as ethyl acetate, butyl acetate, diethyl malonate, etc., amides such as N-methylpyrrolidone, N,N-dimethylformamide, etc., ethers such as diethyl ether, ethyl cyclopentyl ether, tetrahydrofuran, dioxane, etc., aromatic hydrocarbons such as toluene, xylene, etc., aliphatic hydrocarbons such as hexane, heptane, octane, decahydronaphthalene, etc., halogenated hydrocarbons such as chloroform, dichloromethane, methylene chloride, ethylene chloride, etc., nitrile-based solvents such as acetonitrile, propionitrile, etc., dimethyl sulfoxide, dimethyl sulfamide, and the like. With respect to organic solvents to be used, one type may be used singly, or two or more types may be combined in any manner and used. The reaction temperature which can be employed is, for example, in the range of −10° C. to 200° C., preferably in the range of 0° C. to 150° C., and more preferably in the range of 5° C. to 120° C. The reaction time which can be employed is, for example, 5 minutes or more and 20 hours or less, 10 minutes or more and 15 hours or less, or 30 minutes or more and 12 hours or less.

It is preferable to use the sulfonyl fluoride compound (B′), the silylating agent (C′) and the basic compound (D′) in excessive amounts, with respect to the N-hydroxy compound (A′). For example, it is preferable to use the sulfonyl fluoride compound (B′) in an amount of 1.1 mol or more and 2.5 mol or less, the silylating agent (C′) in an amount of 1.1 mol or more and 2.5 mol or less, and the basic compound (D′) in an amount of 1.1 mol or more and 2.5 mol or less, with respective to 1.0 mol of the N-hydroxy compound (A′).

According to such a manufacturing method, the N-organosulfonyloxy compound can be efficiently obtained, and the obtained N-organosulfonyloxy compound can be used as an acid generator for a chemically amplified positive-type photosensitive resin composition or as a compound used in various fields such as pharmaceuticals.

### EXAMPLES

The present invention will be described in more detail below by way of Examples, but the present invention is not limited to these Examples.

### <N-Hydroxy Compound (A′)>

As the N-hydroxy compound, A1, A3 to A8, and A2 prepared according to the following method were used.

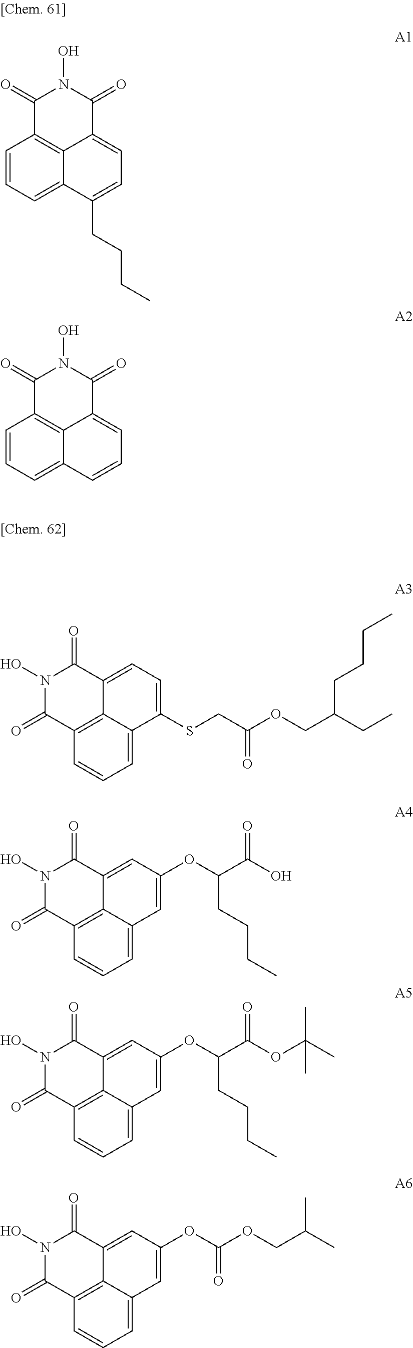

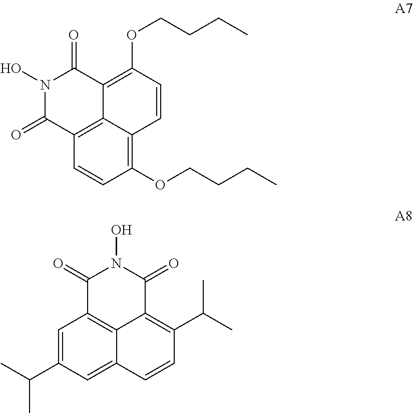

### [Preparation of A1]

Under a nitrogen atmosphere, 534 g of tetrahydrofuran cooled to −70° C. and 0.24 g of a 15% 1-butyllithium hexane solution were stirred, and a suspension of 203 g of zinc bromide and 640 g of tetrahydrofuran was added dropwise at a rate at which the temperature of the reaction system did not rise above −60° C. Thereafter, the reaction system was returned to 10° C., stirred for 1 hour, and a butylzinc reagent was prepared. The butylzinc reagent was added dropwise to a mixture of 100 g of 4-bromonaphthalic anhydride, 5.88 g of [1,1′-bis(diphenylphosphino) ferrocene] palladium (II) dichloride dichloromethane adduct, and 534 g of tetrahydrofuran under a nitrogen atmosphere, and the mixture was stirred at room temperature for 1 hour. 1,000 g of water was added to this, and the organic layer was separated and concentrated to obtain a solid phase. To this solid phase, 532 g of toluene and 100 g of silica gel were added, followed by stirring, and then the solid phase was filtered off. To the solid phase obtained by concentrating the filtrate, 333 g of methanol was added and heated to obtain a solution. A filtrate obtained by filtration of the solution was cooled to perform crystallization. The obtained crystals were washed with isopropyl alcohol and dried under vacuum to obtain 48.0 g of pale-yellow solid (4-butylnaphthalic anhydride). 6.10 g of the 4-butylnaphthalic anhydride obtained above was suspended in 36.0 g of dimethylformamide, 2.00 g of NH₂OH—HCl was added at room temperature, 2.30 g of a 50% aqueous sodium hydroxide solution was added dropwise, and the mixture was stirred for 5 hours. To this was added 24.0 g of water and 0.63 g of a 20% hydrochloric acid solution, and the mixture was further stirred for 2 hours. The precipitate was collected by filtration, washed with a mixture of methanol and water, and then dried under vacuum at 50° C., to obtain 3.17 g of the above A1 (yield: 49%).

### [Preparation of A3]

28 g of 4-bromo-1,8-naphthalic anhydride (manufactured by Tokyo Kasei Kogyo Co., Ltd.) and 11 g of triethylamine were dispersed in 400 g of acetonitrile in an Erlenmeyer flask, and then 22 g of 2-ethylhexyl thioglycolate (manufactured by Tokyo Kasei Kogyo Co., Ltd.) was charged and reacted at 75° C. for 6 hours. Then, 18 g of 50% hydroxylamine aqueous solution (manufactured by Tokyo Chemical Industry Co., Ltd.) was added dropwise and reacted at room temperature for 2 hours. After completion of the reaction, the reaction solution was charged into ion-exchanged water, and then hydrochloric acid was charged until pH5. After stirring for a while, the precipitate was collected by filtration, and dried under reduced pressure at 70° C., thereby obtaining 11.4 g of A3 described above, as a pale-yellow solid (yield: 52%).

### [Preparation of A4]

The same procedure as in [Preparation of A3] was carried out except that 28 g of 4-bromo-1,8-naphthalic anhydride was changed to 21 g of 3-hydroxy-1,8-naphthalic anhydride (manufactured by Tokyo Chemical Industry Co., Ltd.), 11 g of triethylamine was changed to 27 g of potassium carbonate, and 22 g of 2-ethylhexyl thioglycolate was changed to 21 g of 2-bromohexanoic acid (manufactured by Tokyo Chemical Industry Co., Ltd.), thereby obtaining 7.5 g of A4 described above (yield: 45%).

### [Preparation of A5]

After dissolving 40 g of 2-bromohexanoic acid, 4 g of dimethylaminopyridine, and 80 g of tert-butyl alcohol in 800 g of dichloromethane in an Erlenemeyer flask, 50 g of 1-(3-dimethylaminopropyl)-3-ethylcarbodiimide hydrochloride was charged and reacted at room temperature for 3 hours. Then, after washing with a 1% aqueous hydrochloric acid solution, the solvent was removed under reduced pressure to obtain tert-butyl bromohexanoate. 21 g of 3-hydroxy-1,8-naphthalic anhydride and 27 g of potassium carbonate were dispersed in 400 g of acetonitrile in an Erlenemeyer flask, and then 40 g of the above-obtained tert-butyl bromohexanoate was charged and reacted at 75° C. for 6 hours. Then, after filtering off the potassium carbonate, 18 g of a 50% aqueous hydroxylamine solution was added dropwise and reacted at room temperature for 2 hours. After completion of the reaction, the reaction solution was charged into ion-exchanged water, and then hydrochloric acid was charged until pH5. After stirring for a while, the precipitate was collected by filtration, and dried under reduced pressure at 70° C., thereby obtaining 10 g of A5 described above (yield: 51%).

### [Preparation of A6]

5.5 g of 3-hydroxy-1,8-naphthalic anhydride and 5.9 g of di-tert-butyl dicarbonate (manufactured by Tokyo Chemical Industry Co., Ltd.) were dispersed in 32 g of acetonitrile, and 2.2 g of pyridine was added thereto and stirred at 50° C. for 2 hours. After cooling to room temperature, the precipitate was charged into water and filtered off to obtain a white solid. This white solid was washed with water and dried to obtain 6.5 g of 3-isobutoxycarbonyloxy-1,8-naphthalic anhydride. 6.5 g of the obtained 3-isobutoxycarbonyloxy-1,8-naphthalic anhydride was dissolved in 137 g of acetonitrile, 2.0 g of a 50% aqueous hydroxylamine solution was added thereto, and the resulting mixture was stirred at room temperature for 2 hours. The reaction solution was charged into water and the precipitate was filtered off to obtain a white solid. This white solid was washed with water and dried to obtain 3.7 g of A6 described above (yield: 55%).

### [Preparation of A7]

43.4 g of potassium carbonate was dispersed in 168 g of acetone, and 8.4 g of 1,6-dihydroxynaphthalene (manufactured by Tokyo Chemical Industry Co., Ltd.) and 38.8 g of 1-iodobutane were added and stirred at 50° C. for 20 hours. After cooling to room temperature, 15 g of methyl butyl ether was added to the filtrate, the resulting organic layer was washed with water, and the solvent was distilled off to obtain 14.3 g of 1,6-dibutoxynaphthalene. Using the obtained 1,6-dibutoxynaphthalene as a raw material, 3,6-dibutoxyacenaphthenequinone was synthesized according to the method described in the document (Helv. Chem. Acta, 1921, 342.), and 10 g of potassium peroxymonosulfate (double salt) was added under a nitrogen atmosphere in one portion to 29 g of a methanol dispersion of 2.0 g of the 3,6-dibutoxyacenaphthenequinone. The dispersion was refluxed for one day while strongly stirring. After completion of the reaction, the reaction mixture was cooled to room temperature and charged into a large amount of water. The obtained solid was filtered and dried under reduced pressure to obtain 1.6 g of a compound. 1.6 g of the obtained compound was dispersed in 20 g of acetonitrile, and 2.0 g of a 50% aqueous hydroxylamine solution was added thereto, followed by stirring for one day at 50° C. The reaction solution was charged into a dilute aqueous hydrochloric acid solution, and the precipitate was filtered off and washed with water and dried to obtain 1.0 g of the A7 described above (yield: 60%).

### [Preparation of A8]

The same operation as in [Preparation of A7] was carried out, except that 1,6-dibutoxynaphthalene was changed to 2,6-diisopropylnaphthalene (manufactured by Tokyo Kasei Kogyo Co., Ltd.), and 2.0 g of 3,6-dibutoxyacenaphthenequinone was changed to 2.0 g of 3,7-diisopropylacenaphthenequinone to obtain 1.0 g of A8 described above (yield 57%).

### <Sulfonyl Fluoride Compound (B′)>

As the sulfonyl fluoride compound (B′), B1 described below and B2 to B4 prepared according to the following method were used.

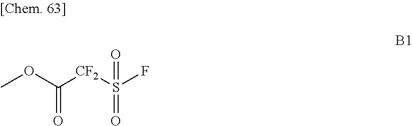

### [Preparation of B2]

8.9 g of 2-cyclohexylethanol and 14.8 g of the following carboxylic acid were stirred in 80.0 g of toluene in the presence of 7.5 g of sulfuric acid under reflux for 2 hours. Thereafter, the reaction solution was washed with purified water and the solvent was distilled off, thereby obtaining the following B2 as a viscous liquid.

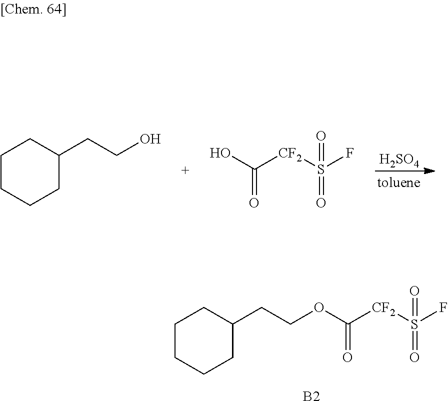

### [Preparation of B3]

The same procedure as in [Preparation of B2] was carried out, except that 2-cyclohexylethanol was changed to 1-adamantaneethanol to obtain B3 described below.

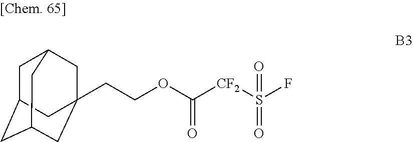

### [Preparation of B4]

The same procedure as in [Preparation of B2] was carried out, except that 2-cyclohexylethanol was changed to the following alcohol to obtain B4.

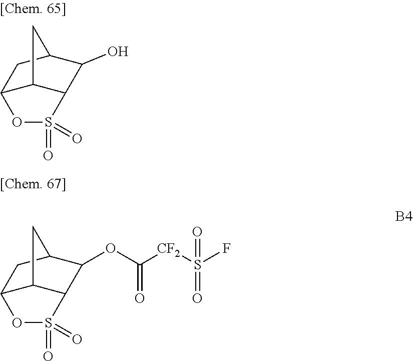 [SMILES: O=S1(=O)OC2CC3CC2C1C3O ; O=C(OC1C2CC3OS(=O)(=O)C1C3C2)C(F)(F)S(=O)(=O)F]

### Preparation of Acid Generator (A)

### Preparation Example 1

To a mixture obtained by mixing 1.94 g of A1 as the N-hydroxy compound (A′) and 42.4 g of dichloromethane, 1.12 g of N-ethyldiisopropylamine as the basic compound (D′) was added, 1.02 g of trimethylsilyl chloride as the silylating agent (C′) was added, and then 1.54 g of B1 as the sulfonyl fluoride compound (B′) was added and the obtained mixture was stirred at room temperature for 12 hours. Thereafter, the reaction solution was washed with a 1% hydrochloric acid solution, and washed with purified water, and the solvent was distilled off to obtain 2.71 g of PAG-1 as a viscous liquid (yield: 85%). Measurement results of ¹H-NMR of the following compound PAG-1 are as follows. ¹H-NMR (CDCl₃): δ=8.41-8.80 (m, 3H), 7.85 (m, 1H), 7.64 (d, 1H), 4.00 (s, 3H), 3.31 (t, 2H), 1.80 (m, 2H), 1.49 (m, 2H), 1.10 (t, 3H)

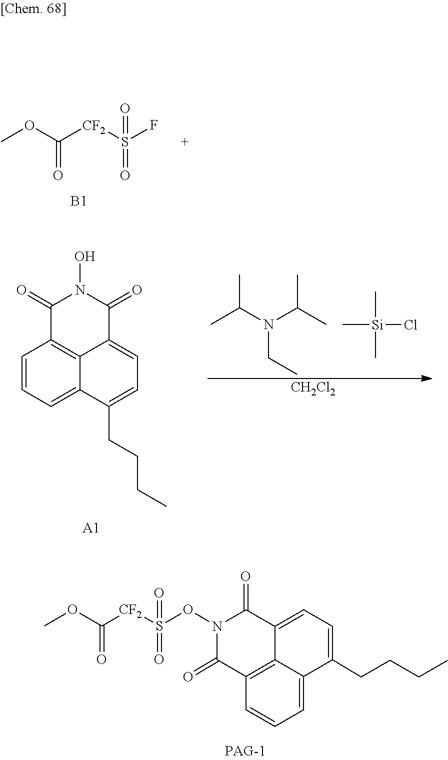

### Preparation Example 2

The same procedure as in Preparation Example 1 was carried out, except that B2 was used instead of B1. The obtained compound was confirmed to be the following compound PAG-2 by the same analysis as in Preparation Example 1. The yield was 83%.

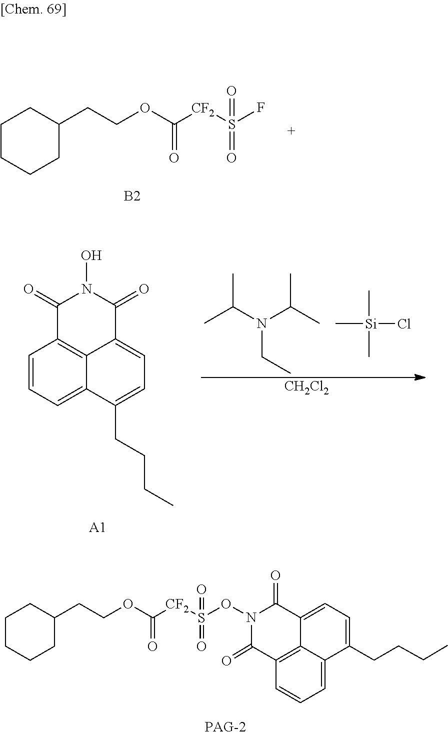

### Preparation Example 3

The same procedure as in Preparation Example 1 was carried out, except that B3 was used instead of B1. The obtained compound was confirmed to be the following compound PAG-3 by the same analysis as in Preparation Example 1. The yield was 90%.

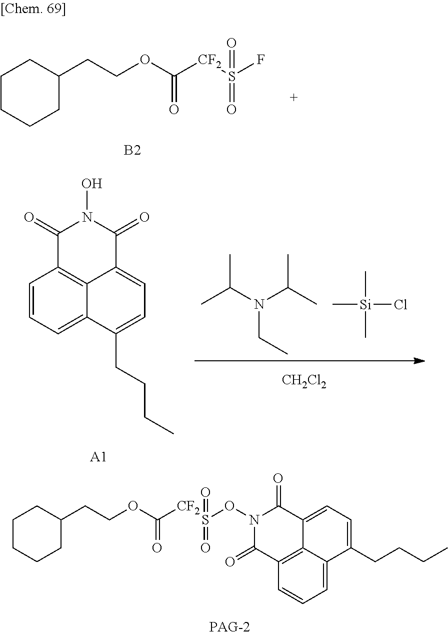

### Preparation Example 4

The same procedure as in Preparation Example 1 was carried out except that B4 was used instead of B1. The obtained compound was confirmed to be the following compound PAG-4 by the same analysis as in Preparation Example 1. The yield was 85%.

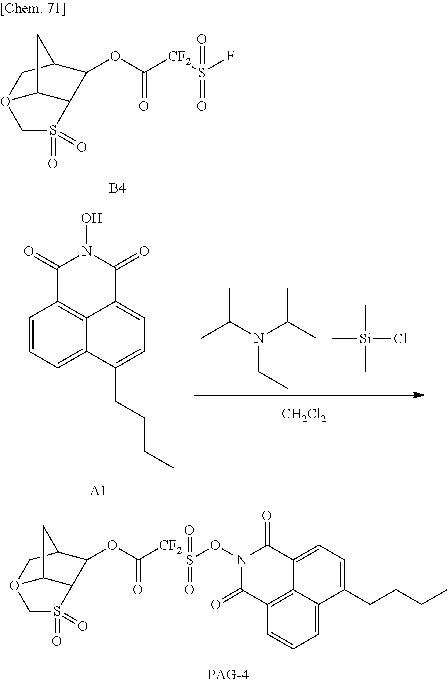

### Preparation Example 5

The same procedure as in Preparation Example 1 was carried out, except that A2 was used instead of A1. The obtained compound was confirmed to be the following compound PAG-7 by the same analysis as in Preparation Example 1. The yield was 80%.

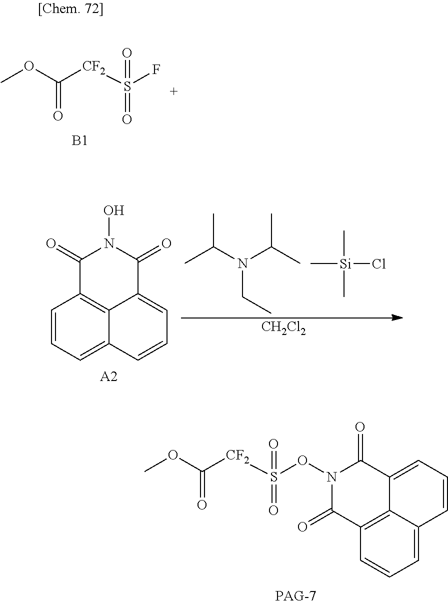

### Preparation Example 6

The same procedure as in Preparation Example 1 was carried out, except that B4 was used instead of B1 and A2 was used instead of A1. The obtained compound was confirmed to be the following compound PAG-8 by the same analysis as in Preparation Example 1. The yield was 75%.

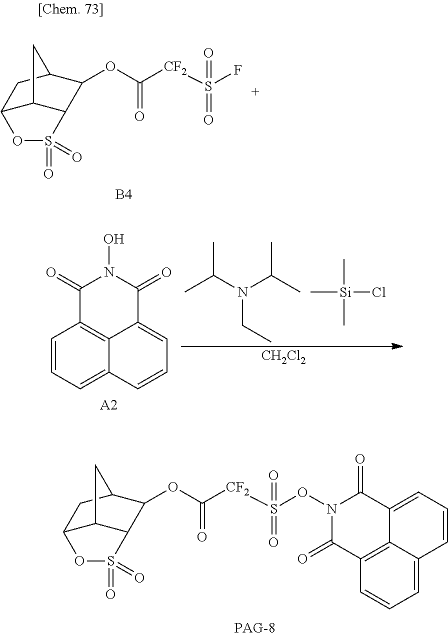

### Preparation Example 7

The same procedure as in Preparation Example 1 was carried out, except that A3 was used instead of A1. The obtained compound was confirmed to be the following compound PAG-9 by the same analyses as in Preparation Example 1. The yield was 84%.

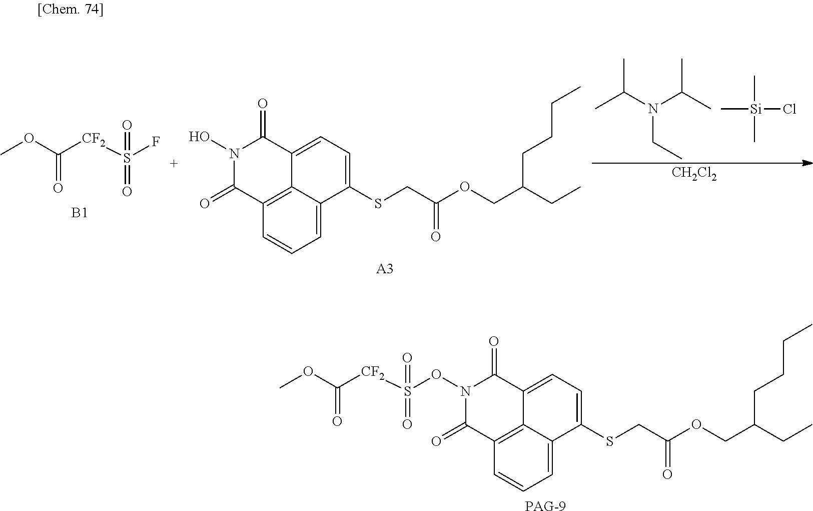

### Preparation Example 8

The same procedure as in Preparation Example 2 was carried out, except that A3 was used instead of A1. The obtained compound was confirmed to be the following compound PAG-10 by the same analysis as in Preparation Example 1. The yield was 86%.

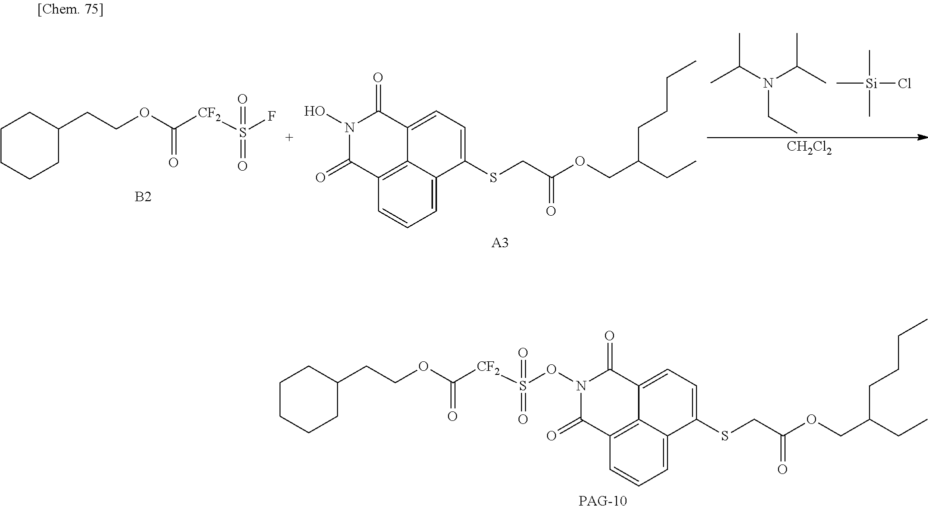

### Preparation Example 9

The same procedure as in Preparation Example 3 was carried out, except that A3 was used instead of A1. The obtained compound was confirmed to be the following compound PAG-11 by the same analysis as in Preparation Example 1. The yield was 87%.

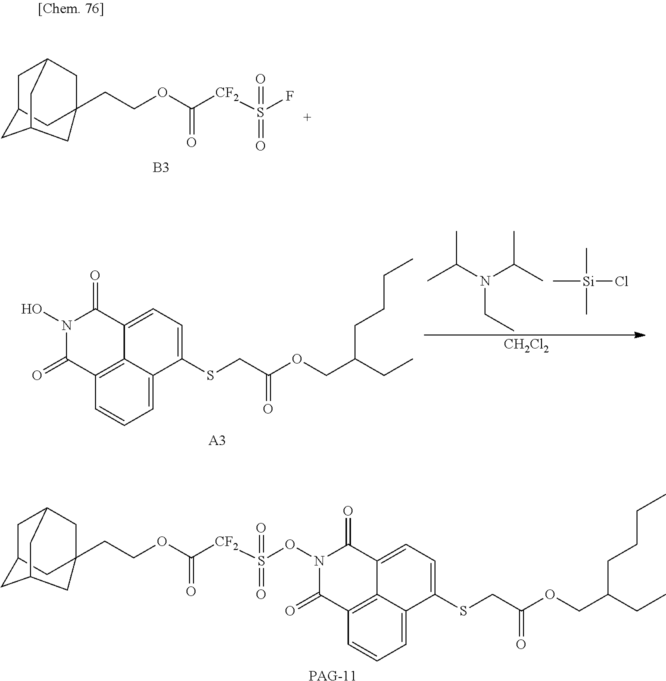

### Preparation Example 10

The same procedure as in Preparation Example 4 was carried out, except that A3 was used instead of A1. The obtained compound was confirmed to be the following compound PAG-12 by the same analysis as in Preparation Example 1. The yield was 80%.

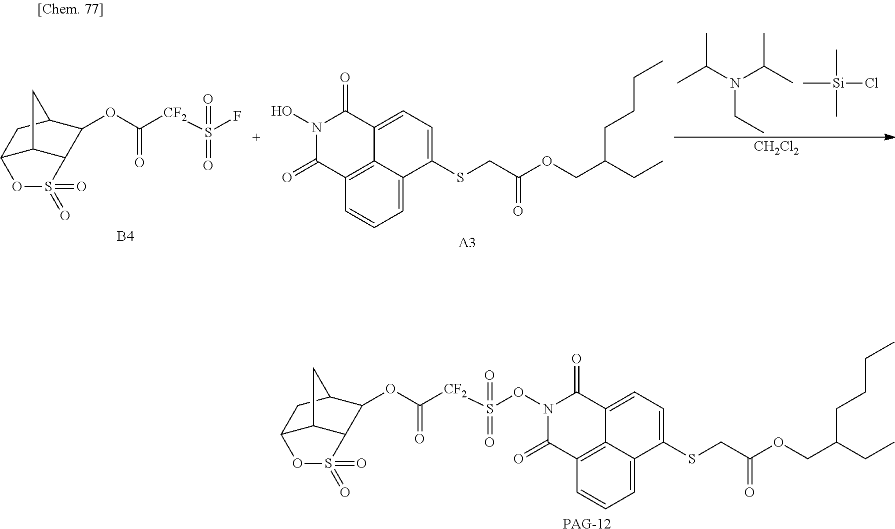

### Preparation Example 11

The same procedure as in Preparation Example 1 was carried out, except that A4 was used instead of A1. The obtained compound was confirmed to be the following compound PAG-13 by the same analysis as in Preparation Example 1. The yield was 87%.

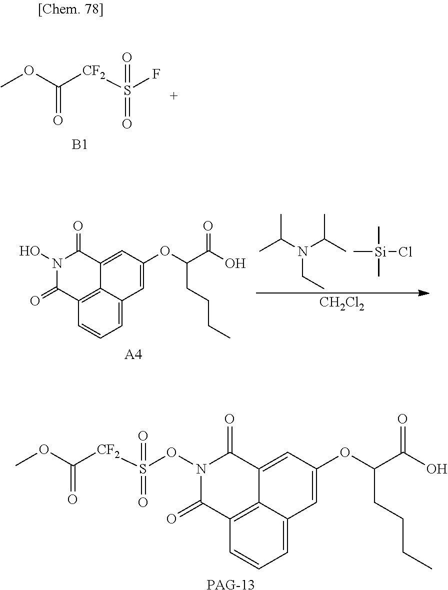

### Preparation Example 12

The same procedure as in Preparation Example 2 was carried out, except that A4 was used instead of A1. The obtained compound was confirmed to be the following compound PAG-14 by the same analysis as in Preparation Example 1. The yield was 80%.

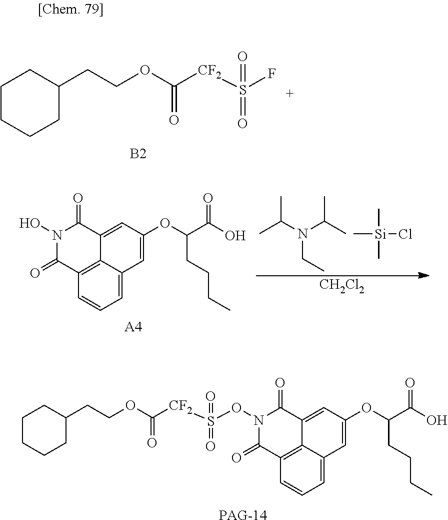

### Preparation Example 13

The same procedure as in Preparation Example 3 was carried out, except that A4 was used instead of A1. The obtained compound was confirmed to be the following compound PAG-15 by the same analysis as in Preparation Example 1. The yield was 79%.

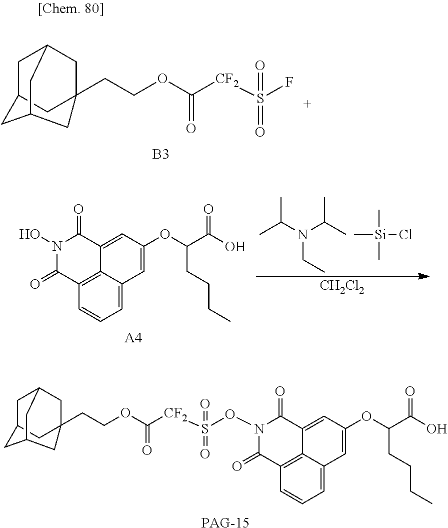

### Preparation Example 14

The same procedure as in Preparation Example 4 was carried out, except that A4 was used instead of A1. The obtained compound was confirmed to be the following compound PAG-16 by the same analysis as in Preparation Example 1. The yield was 76%.

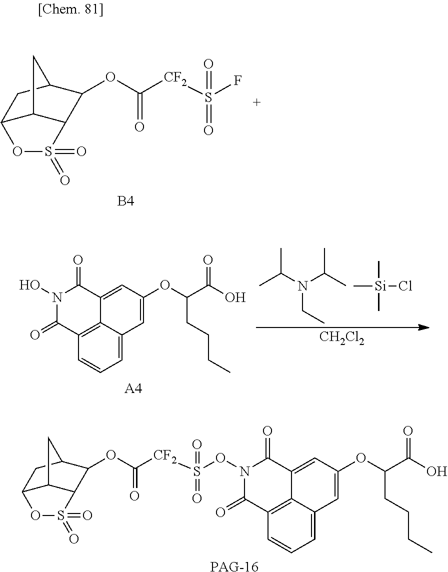

### Preparation Example 15

The same procedure as in Preparation Example 1 was carried out, except that A5 was used instead of A1. The obtained compound was confirmed to be the following compound PAG-17 by the same analysis as in Preparation Example 1. The yield was 82%.

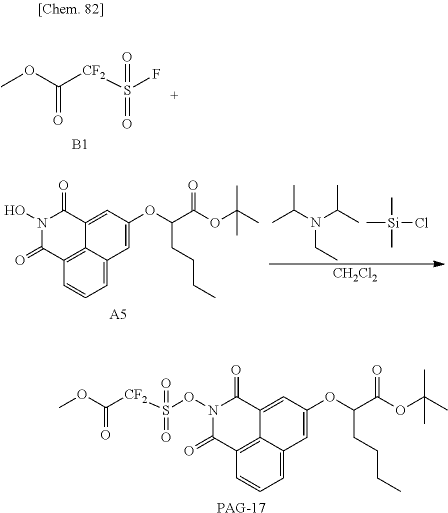

### Preparation Example 16

The same procedure as in Preparation Example 2 was carried out, except that A5 was used instead of A1. The obtained compound was confirmed to be the following compound PAG-18 by the same analysis as in Preparation Example 1. The yield was 85%.

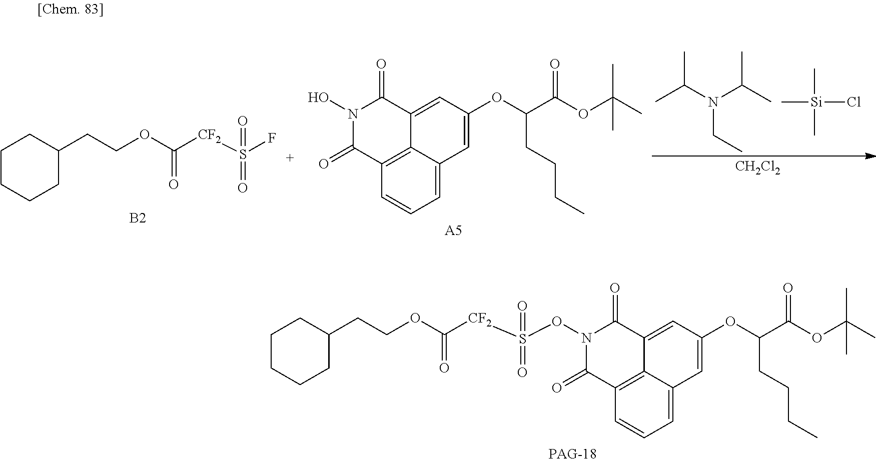

### Preparation Example 17

The same procedure as in Preparation Example 3 was carried out, except that A5 was used instead of A1. The obtained compound was confirmed to be the following compound PAG-19 by the same analysis as in Preparation Example 1. The yield was 86%.

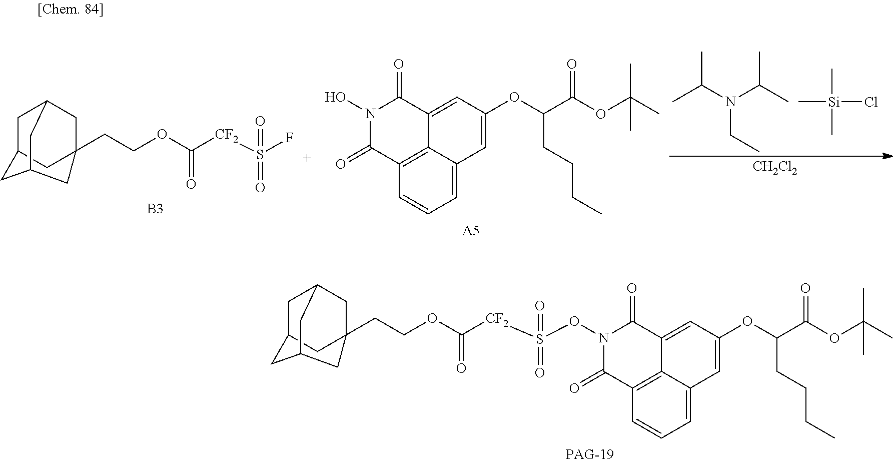

### Preparation Example 18

The same procedure as in Preparation Example 4 was carried out, except that A5 was used instead of A1. The obtained compound was confirmed to be the following compound PAG-20 by the same analysis as in Preparation Example 1. The yield was 81%.

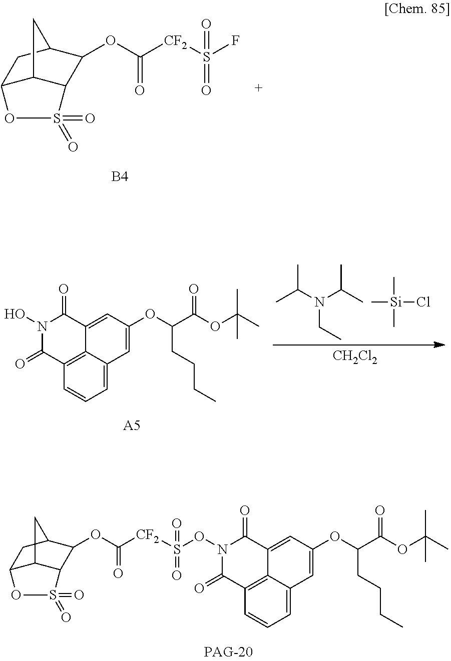

### Preparation Example 19

The same procedure as in Preparation Example 1 was carried out except that A6 was used instead of A1. The obtained compound was confirmed to be the following compound PAG-21 by the same analysis as in Preparation Example 1. The yield was 80%.

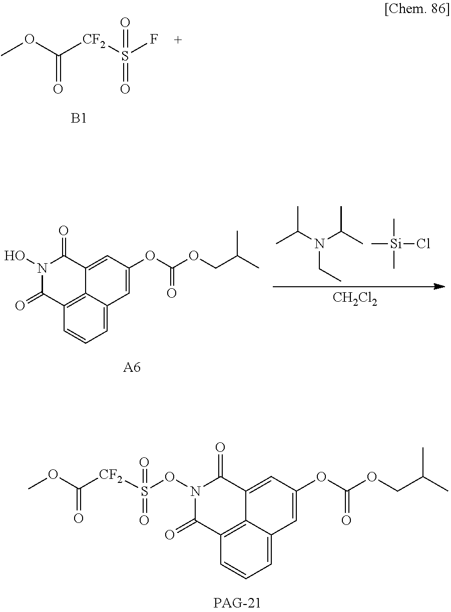

### Preparation Example 20

The same procedure as in Preparation Example 2 was carried out, except that A6 was used instead of A1. The obtained compound was confirmed to be the following compound PAG-22 by the same analysis as in Preparation Example 1. The yield was 83%.

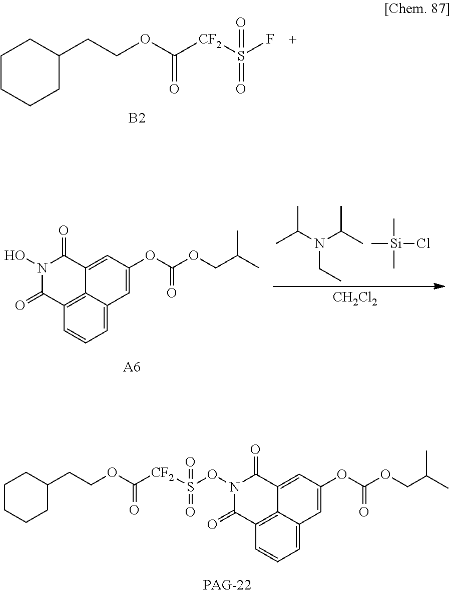

### Preparation Example 21

The same procedure as in Preparation Example 3 was carried out, except that A6 was used instead of A1. The obtained compound was confirmed to be the following compound PAG-23 by the same analysis as in Preparation Example 1. The yield was 83%.

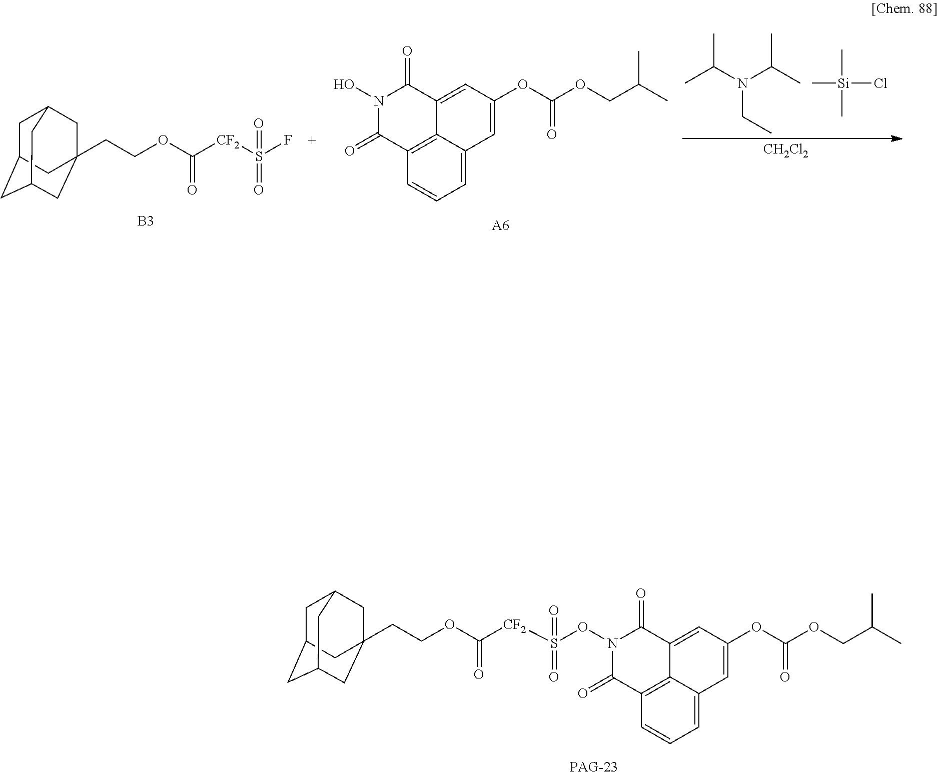

### Preparation Example 22

The same procedure as in Preparation Example 4 was carried out, except that A6 was used instead of A1. The obtained compound was confirmed to be the following compound PAG-24 by the same analysis as in Preparation Example 1. The yield was 79%.

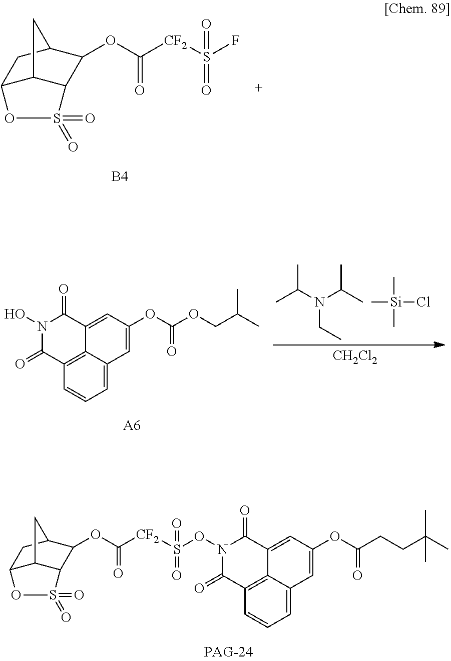

### Preparation Example 23

The same procedure as in Preparation Example 1 was carried out, except that A7 was used instead of A1. The obtained compound was confirmed to be the following compound PAG-25 by the same analysis as in Preparation Example 1. The yield was 86%.

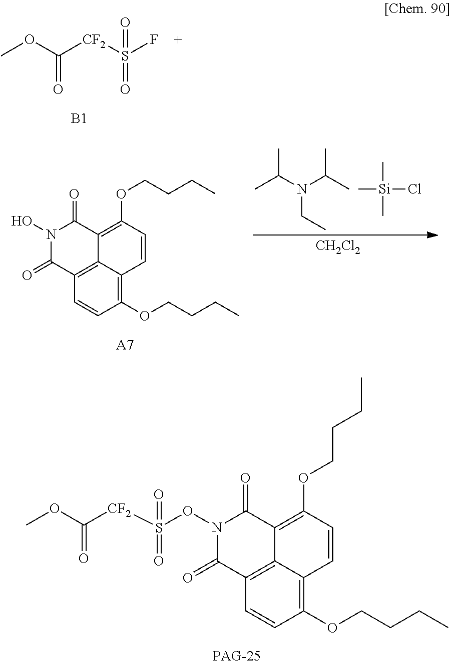

### Preparation Example 24

The same procedure as in Preparation Example 2 was carried out, except that A7 was used instead of A1. The obtained compound was confirmed to be the following compound PAG-26 by the same analysis as in Preparation Example 1. The yield was 84%.

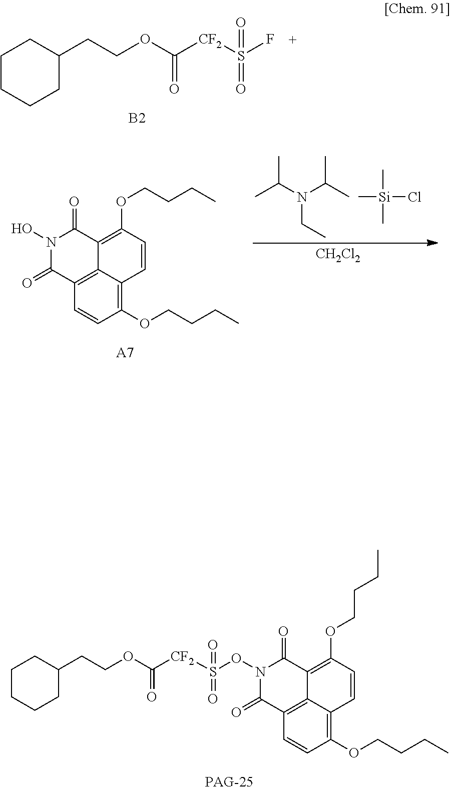

### Preparation Example 25

The same procedure as in Preparation Example 3 was carried out, except that A7 was used instead of A1. The obtained compound was confirmed to be the following compound PAG-27 by the same analysis as in Preparation Example 1. The yield was 91%.

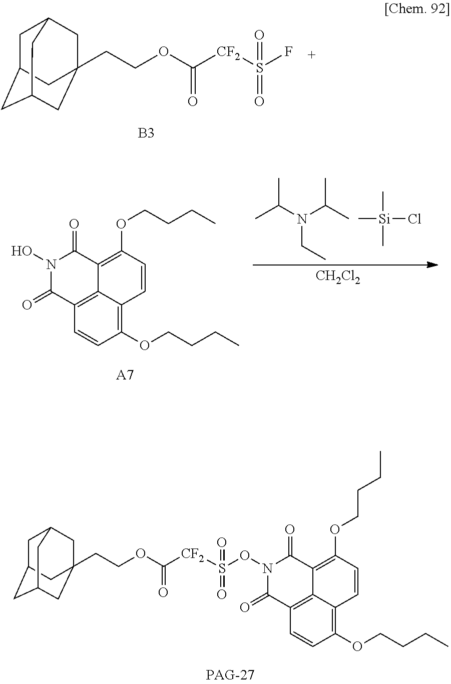

### Preparation Example 26

The same procedure as in Preparation Example 4 was carried out, except that A7 was used instead of A1. The obtained compound was confirmed to be the following compound PAG-28 by the same analysis as in Preparation Example 1. The yield was 82%.

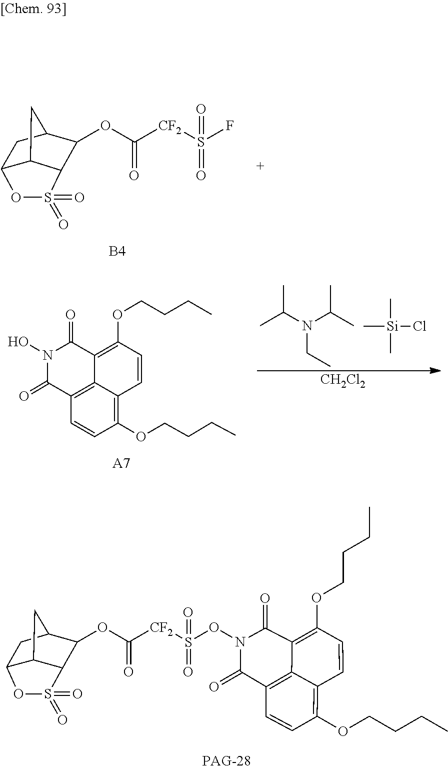

### Preparation Example 27

The same procedure as in Preparation Example 1 was carried out except that A8 was used instead of A1. The obtained compound was confirmed to be the following compound PAG-29 by the same analyses as in Preparation Example 1. The yield was 88%.

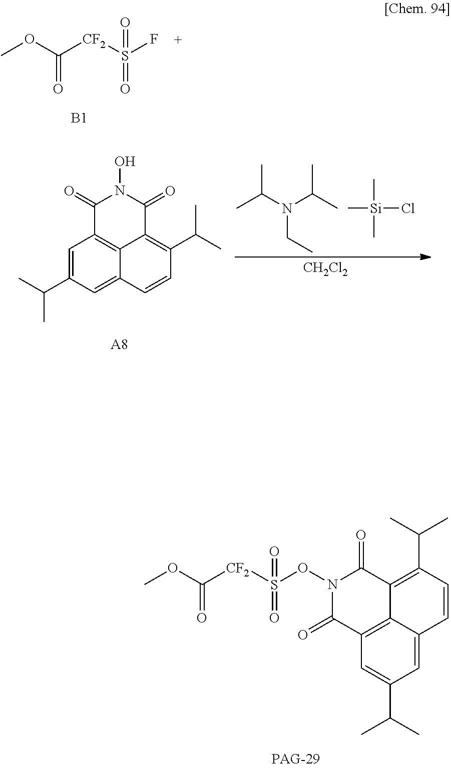

### Preparation Example 28

The same procedure as in Preparation Example 2 was carried out, except that A8 was used instead of A1. The obtained compound was confirmed to be the following compound PAG-30 by the same analysis as in Preparation Example 1. The yield was 86%.

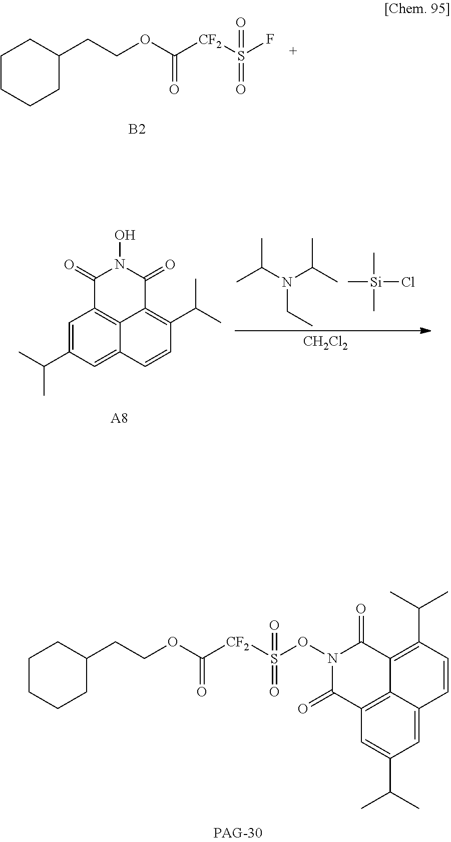

### Preparation Example 29

The same procedure as in Preparation Example 3 was carried out, except that A8 was used instead of A1. The obtained compound was confirmed to be the following compound PAG-31 by the same analysis as in Preparation Example 1. The yield was 93%.

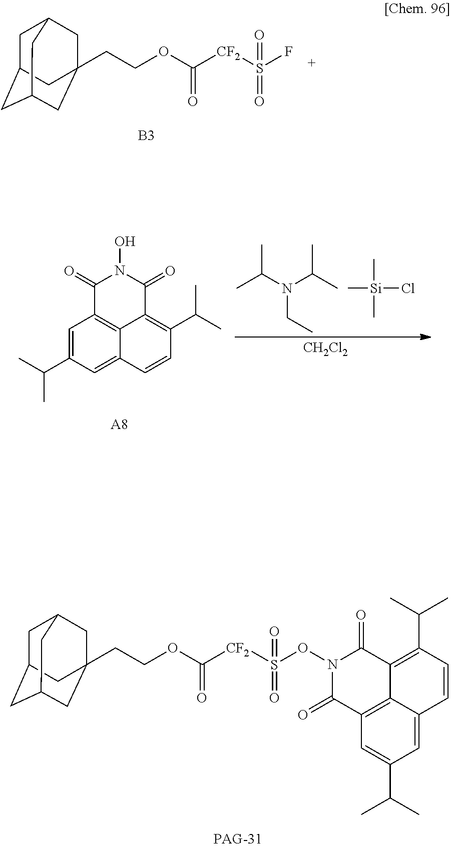

### Preparation Example 30

The same procedure as in Preparation Example 4 was carried out, except that A8 was used instead of A1. The obtained compound was confirmed to be the following compound PAG-32 by the same analysis as in Preparation Example 1. The yield was 84%.

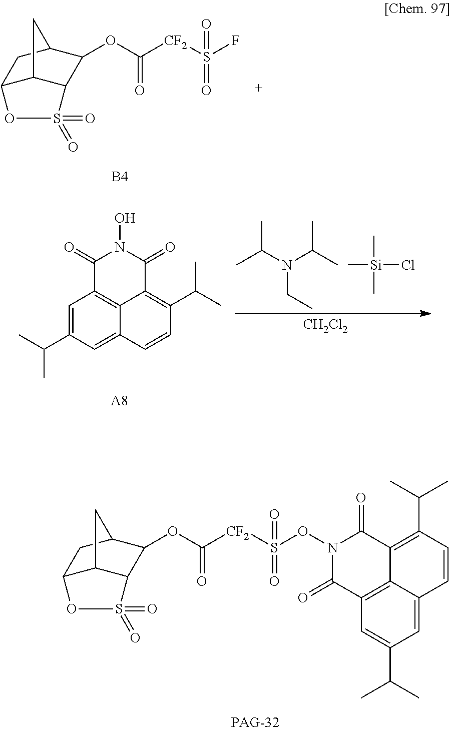

### Examples 1 to 28, and Comparative Examples 1 to 4

In Examples 1 to 28 and Comparative Examples 1 to 4, PAG-1 to PAG-4, PAG-7 to PAG-32, PAG-5, and PAG-6 were used as the acid generator (A).

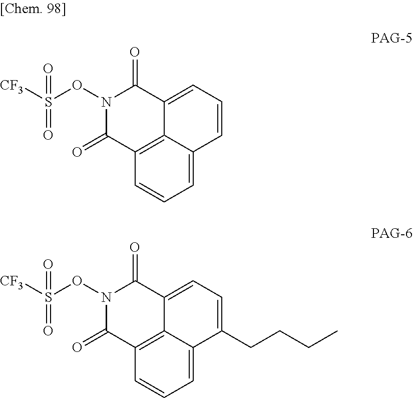 [SMILES: O=C1c2cccc3cccc(c23)C(=O)N1OS(=O)(=O)C(F)(F)F ; CCCCc1ccc2c3c(cccc13)C(=O)N(OS(=O)(=O)C(F)(F)F)C2=O]

In Examples 1 to 28 and Comparative Examples 1 to 4, the following Resins-A were used as the resin whose solubility in alkali increases under action of an acid (the resin (B)). The number at the lower right of the parentheses in each constituent unit in the following structural formulae represents the content (% by mass) of the constituent unit in each resin. Resin-A has a mass average molecular weight Mw of 42,000.

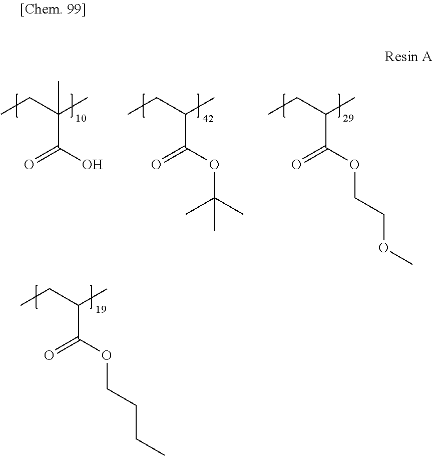

As the alkali-soluble resin (D), the following Resin-B (polyhydroxystyrene resin), and Resin-C (novolac resin (homo-condensate of m-cresol) were used. The number at the lower right of the parentheses in each constituent unit in the following structural formulae represents the content (% by mass) of the constituent unit in each resin. The resin Resin-B has a mass average molecular weight (Mw) of 2,500, and the dispersivity (Mw/Mn) of 2.4. The resin Resin-C has a mass average molecular weight (Mw) of 8000.

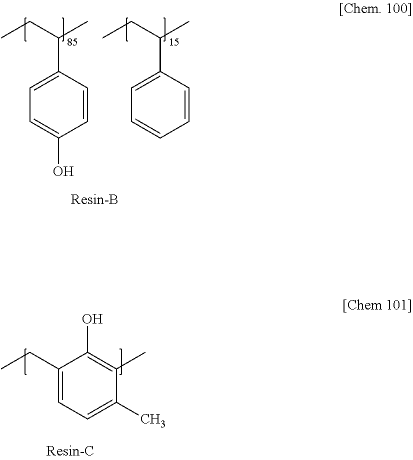

As the sulfur-containing compound (E), the following sulfur-containing compounds T1 and T2 were used.

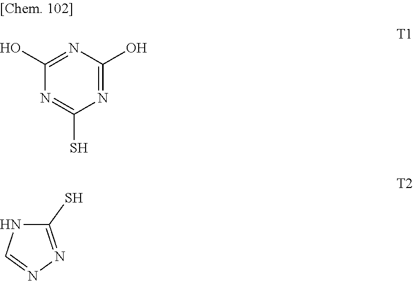 [SMILES: Oc1nc(O)nc(S)n1 ; Sc1nnc[nH]1]

As the acid diffusion controlling agent (F), di-t-butylpyridine (Q¹) was used.

The acid generator (A), the resin (B), the alkali-soluble resin (D), the sulfur-containing compound (E), the acid diffusion controlling agent (F), and the surfactant (BYK310, manufactured by Byk Chemie Co., Ltd.) of the types and amounts described in Table 1 and Table 2 were dissolved in propylene glycol monomethyl ether acetate (PGMEA) to obtain the photosensitive resin compositions of the Examples and Comparative Examples. Note here that the surfactant (BYK310, manufactured by BYK) was added so that the amount thereof was 0.05 parts by mass with respect to the total amount of the resin (B) and the alkali-soluble resin (D). The photosensitive resin compositions of Examples 1 to 28 and Comparative Examples 1 to 4 were prepared so that the solid concentration was 35% by mass.

Using the obtained photosensitive resin compositions, mask linearity was evaluated according to the following method. The results are shown in Tables 1 and 2.

### [Evaluation of Mask Linearity]

Glass substrates each having a diameter of 500 mm and each being provided with a copper layer by sputtering on the surface were prepared, and each photosensitive resin composition of each Example and each Comparative Example was applied to the copper layer of each substrate to form a photosensitive layer having a film thickness of 5 μm. The photosensitive layer was then pre-baked for 4 minutes at 120° C. After pre-baking, by using a mask with a line and space pattern having a line width of 2 μm and a space width of 2 μm and an exposure device Canon FPA-5510iV (manufactured by Canon Co., Ltd.), pattern-exposure was performed with ultraviolet rays having a wavelength of 365 nm. The substrate was then placed on a hot plate for post-exposure heating (PEB) at 90° C. for 4 minutes. Thereafter, a 2.38% by weight aqueous solution of tetramethylammonium hydroxide (developer, NMD-3, manufactured by Tokyo Ohka Kogyo Co., Ltd.) was added dropwise onto the exposed photosensitive layer and then the substrate was left to stand at 23° C. for 30 seconds. This operation was repeated twice in total. Subsequently, the surface of the resist pattern was washed (rinsed) with running water, and blown with nitrogen to obtain a resist pattern. In the resist pattern formation described above, a line and space (LS) pattern was formed in the same manner as in the formation of the resist pattern described above, by appropriately shifting the focal point upwardly and downwardly with an exposure dose (J/m²) at which the line and space (LS) pattern was formed. At this time, a depth of focus (DOF, unit: μm) in which the LS pattern could be formed within a range of a dimensional change rate of the target dimension±10% (i.e., 1.80 to 2.20 μm) was determined and the depth of focus was used as an evaluation criterion for mask linearity. The DOF is a range of depth of focus in which a resist pattern having a dimensional deviation from the target dimension being within a predetermined range can be formed when the exposure focus is moved upwardly or downwardly with the same exposure dose, i.e., a range of depth of focus in which a resist pattern faithful to the mask pattern can be obtained. A larger DOF is more preferable. That is, the larger the DOF value, the better the mask linearity. The results are shown in Table 1 and Table 2.

### [Evaluation of Solubility of Acid Generator]

For each acid generator used in Examples 1 to 28 and Comparative Examples 1 to 4, the maximal concentration dissolved in propylene glycol monomethyl ether acetate (PGMEA) was measured. The results are shown in Table 1 and Table 2.

TABLE 1

| | | Resin (B) and | Sulfur- | Acid diffusion | | |
|---|---|---|---|---|---|---|
| | Acid | alkali-soluble | containing | controlling | Evaluation | |
| | generator | resin | compound | agent | | Solvent |
| | (A) | (D) | (E) | (F) | | solubility |
| | Type/parts | Type/parts | Type/parts | Type/parts | DOF | of acid |
| | by mass | by mass | by mass | by mass | (μm) | generator |
| Example 1 | PAG-1/1.2 | ResinA/35 | T1/0.05 | Q1/0.05 | 16.0 | Greater than |
| | | ResinB/10 | T2/0.05 | | | 20% by mass |
| Example 2 | PAG-2/1.4 | ResinC/55 | | | 18.0 | Greater than |
| | | | | | | 20% by mass |
| Example 3 | PAG-3/1.5 | | | | 20.0 | Greater than |
| | | | | | | 20% by mass |
| Example 4 | PAG-4/1.7 | | | | 20.0 | Greater than |
| | | | | | | 20% by mass |
| Comparative | PAG-5/0.9 | | | | 4.0 | Less than |
| Example 1 | | | | | | 1% by mass |
| Comparative | PAG-6/1.0 | | | | 6.0 | Greater than |
| Example 2 | | | | | | 20% by mass |
| Comparative | PAG-7/1.1 | | | | 16.0 | 5% by mass |
| Example 3 | | | | | | |
| Comparative | PAG-8/1.6 | | | | 20.0 | Less than |
| Example 4 | | | | | | 1% by mass |

TABLE 2

| | | Resin (B) and | Sulfur- | Acid diffusion | | |
|---|---|---|---|---|---|---|
| | Acid | alkali-soluble | containing | controlling | Evaluation | |
| | generator | resin | compound | agent | | Solvent |
| | (A) | (D) | (E) | (F) | | solubility |
| | Type/parts | Type/parts | Type/parts | Type/parts | DOF | of acid |
| | by mass | by mass | by mass | by mass | (μm) | generator |
| Example 5 | PAG-9/1.6 | ResinA/35 | T1/0.05 | Q1/0.05 | 16 | Greater than |
| | | ResmB/10 | T2/0.05 | | | 20% by mass |
| Example 6 | PAG-10/1.9 | ResinC/55 | | | 16 | Greater than |
| | | | | | | 20% by mass |
| Example 7 | PAG-11/2.1 | | | | 18 | Greater than |
| | | | | | | 20% by mass |
| Example 8 | PAG-12/2.3 | | | | 18 | Greater than |
| | | | | | | 20% by mass |
| Example 9 | PAG-13/1.4 | | | | 18 | 10% by mass |
| Example 10 | PAG-14/1.7 | | | | 18 | 10% by mass |
| Example 11 | PAG-15/1.9 | | | | 20 | 10% by mass |
| Example 12 | PAG-16/2.1 | | | | 20 | 10% by mass |
| Example 13 | PAG-17/1.6 | | | | 16 | 15% by mass |
| Example 14 | PAG-18/1.9 | | | | 18 | 15% by mass |
| Example 15 | PAG-19/2.1 | | | | 20 | 15% by mass |
| Example 16 | PAG-20/2.2 | | | | 18 | 10% by mass |
| Example 17 | PAG-21/1.4 | | | | 16 | Greater than |
| | | | | | | 20% by mass |
| Example 18 | PAG-22/1.6 | | | | 18 | Greater than |
| | | | | | | 20% by mass |
| Example 19 | PAG-23/1.9 | | | | 20 | Greater than |
| | | | | | | 20% by mass |
| Example 20 | PAG-24/2.0 | | | | 20 | 10% by mass |
| Example 21 | PAG-25/1.5 | | | | 16 | Greater than |
| | | | | | | 20% by mass |
| Example 22 | PAG-26/1.7 | | | | 16 | Greater than |
| | | | | | | 20% by mass |
| Example 23 | PAG-27/1.9 | | | | 18 | Greater than |
| | | | | | | 20% by mass |
| Example 24 | PAG-28/2.1 | | | | 18 | Greater than |
| | | | | | | 20% by mass |
| Example 25 | PAG-29/1.3 | | | | 18 | 10% by mass |
| Example 26 | PAG-30/1.6 | | | | 18 | Greater than |
| | | | | | | 20% by mass |
| Example 27 | PAG-31/1.8 | | | | 20 | Greater than |
| | | | | | | 20% by mass |
| Example 28 | PAG-32/1.9 | | | | 20 | 10% by mass |

According to Examples 1 to 28, the positive-type photosensitive resin compositions each containing the compound represented by the formula (A1) as the acid generator (A), which generates acid when irradiated with an active ray or radiation, formed resist patterns excellent in mask linearity. Further, the solvent solubility of the acid generator (A) was also good.

On the other hand, according to Comparative Examples 1 to 4, it can be seen that, in a case in which the acid generator contained in the positive-type photosensitive resin composition was not a compound represented by the formula (A1), the solvent solubility of the acid generator contained was poor or the mask linearity was poor as compared with Examples 1 to 28.

### Comparative Preparation Example 1

To a mixture of 2.00 g of A2 as the N-hydroxy compound (A′) and 30.0 g of dichloromethane, 1.45 g of N-ethyldiisopropylamine as the basic compound (D′) was added, and then 1.98 g of B1 as the sulfonyl fluoride compound (B′) was added and the resulting mixture was stirred at room temperature for 12 hours. Thereafter, the reaction solution was washed with a 1% hydrochloric acid solution, and washed with purified water, and the solvent was distilled off to obtain 0.17 g of a solid. Structural identification by NMR was attempted on the obtained compound, but it could not be confirmed that the obtained compound was PAG-7.

As shown in Preparation Examples 1 to 6 and Preparation Examples 7 to 30 (Examples) described above, N-organosulfonyloxy compounds can be efficiently manufactured by the method of manufacturing of the present invention. On the other hand, in Comparative Preparation Example 1 in which no silylating agent (C′) was used, the N-organosulfonyloxy compound could not be produced.

1. A chemically amplified positive-type photosensitive resin composition comprising:
an acid generator (A) which generates acid when irradiated with an active ray or radiation, and a resin (B) whose solubility in alkali increases under action of an acid,
wherein the acid generator (A) comprises a compound represented by the following formula (A1): 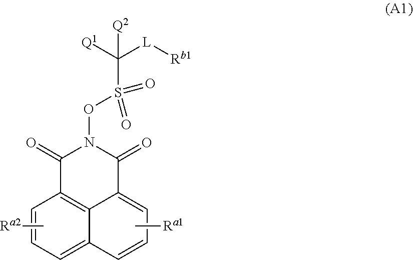
wherein R^{b1} is a hydrocarbon group having 1 or more and 30 or less carbon atoms;
when the hydrocarbon group as R^{b1} contains 1 or more methylene groups, at least a part of the methylene groups may be replaced with a group selected from the group consisting of —O—, —S—, —CO—, —OO—O—, —SO—, —SO₂—, —CR^{b4}R^{b5}—, and —NR^{b6}—;
when the hydrocarbon group as R^{b1} contains a hydrocarbon ring, at least one of the carbon atoms constituting the hydrocarbon ring may be replaced with a heteroatom selected from the group consisting of N, O, P, S, and Se or an atomic group containing the heteroatom;
R^{b4} and R^{b5} are each independently a hydrogen atom or a halogen atom, and at least one of R^{b4} and R^{b5} is a halogen atom;
R^{b6} is a hydrogen atom or a hydrocarbon group having 1 or more and 6 or less carbon atoms;
R^{a1} and R^{a2} are each independently a hydrogen atom, an optionally substituted aliphatic hydrocarbon group having 1 or more and 20 or less carbon atoms, an optionally substituted aromatic group having 5 or more and 20 or less ring constituting atoms, or a group represented by —R^{a3}—R^{a4};
R^{a1} and R^{a2} are not simultaneously hydrogen atoms;
when the aliphatic hydrocarbon group as R^{a1} or R^{a2} contains 1 or more methylene groups, at least a part of the methylene groups may be replaced with a group selected from the group consisting of —O—, —S—, —CO—, —CO—O—, —SO—, —SO₂—, and —NR^{a5}—;
R^{a5} is a hydrogen atom or a hydrocarbon group having 1 or more and 6 or less carbon atoms;
R^{a3} is a methylene group, —O—, —CO—, —CO—O—, —SO—, —SO₂—, or —NR^{a6}—;
R^{a6} is a hydrogen atom or a hydrocarbon group having 1 or more and 6 or less carbon atoms;
R^{a4} is an optionally substituted aromatic group having 5 or more and 20 or less ring constituting atoms, a perfluoroalkyl group having 1 or more and 6 or less carbon atoms, an optionally substituted aralkyl group having 7 or more and 20 or less carbon atoms, or a heteroarylalkyl group having an optionally substituted aromatic heterocyclic group having 5 or more and 20 or less ring constituting atoms;
Q¹ and Q² are each independently a fluorine atom or a perfluoroalkyl group having 1 or more and 6 or less carbon atoms; and
L is an ester bond.

2. The chemically amplified positive-type photosensitive resin composition according to claim 1, wherein the compound represented by the formula (A1) is a compound represented by the following formula (A1-1): 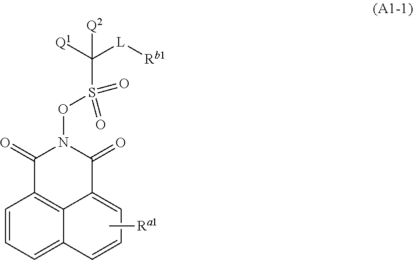
wherein R^{b1}, R^{a1}, Q¹, and Q² are the same as those in the formula (A1).

3. The chemically amplified positive-type photosensitive resin composition according to claim 2, wherein R^{a1} is an optionally substituted aliphatic hydrocarbon group having 1 or more and 20 or less carbon atoms, and
when the aliphatic hydrocarbon group as R^{a1} contains 1 or more methylene groups, at least a part of the methylene groups may be replaced with a group selected from the group consisting of —O—, —S—, —CO—, —CO—O—, —SO—, —SO₂—, and —NR^{a5}—.

4. The chemically amplified positive-type photosensitive resin composition according to claim 1, further comprising an alkali-soluble resin (D).

5. The chemically amplified positive-type photosensitive resin composition according to claim 4, wherein the alkali-soluble resin (D) comprises at least one resin selected from the group consisting of a novolac resin (D1), a polyhydroxystyrene resin (D2), and an acrylic resin (D3).

6. A photosensitive dry film, comprising a substrate film and a photosensitive layer formed on a surface of the substrate film, wherein the photosensitive layer comprises the chemically amplified positive-type photosensitive resin composition according to claim 1.

7. A method of manufacturing a photosensitive dry film, the method comprising applying the chemically amplified positive-type photosensitive resin composition according to claim 1 to a substrate film to form a photosensitive layer.

8. A method of manufacturing a patterned resist film, the method comprising:
laminating a photosensitive layer on a substrate, the photosensitive layer comprising the chemically amplified positive-type photosensitive resin composition according to claim 1;
exposing the photosensitive layer to irradiation with an active ray or radiation in a position-selective manner; and
developing the exposed photosensitive layer.

9. A compound represented by the following formula (A1): 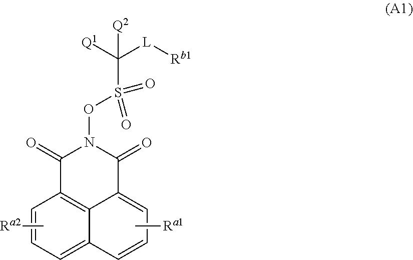
wherein R^{b1} is a hydrocarbon group having 1 or more and 30 or less carbon atoms;
when the hydrocarbon group as R^{b1} contains 1 or more methylene groups, at least a part of the methylene groups may be replaced with a group selected from the group consisting of —O—, —S—, —CO—, —OO—O—, —SO—, —SO₂—, —CR^{b4}R^{b5}—, and —NR^{b6}—;
when the hydrocarbon group as R^{b1} contains a hydrocarbon ring, at least one of the carbon atoms constituting the hydrocarbon ring may be replaced with a heteroatom selected from the group consisting of N, O, P, S, and Se or an atomic group containing the heteroatom;
R^{b4} and R^{b5} are each independently a hydrogen atom or a halogen atom, and at least one of R^{b4} and R^{b5} is a halogen atom;
R^{b6} is a hydrogen atom or a hydrocarbon group having 1 or more and 6 or less carbon atoms;
R^{a1} and R^{a2} are each independently a hydrogen atom, an optionally substituted aliphatic hydrocarbon group having 1 or more and 20 or less carbon atoms, an optionally substituted aromatic group having 5 or more and 20 or less ring constituting atoms, or a group represented by —R^{a3}—R^{a4};
R^{a1} and R^{a2} are not simultaneously hydrogen atoms;
when the aliphatic hydrocarbon group as R^{a1} or R^{a2} contains 1 or more methylene groups, at least a part of the methylene groups may be replaced with a group selected from the group consisting of —O—, —S—, —CO—, —CO—O—, —SO—, —SO₂—, and —NR^{a5}—;
R^{a5} is a hydrogen atom or a hydrocarbon group having 1 or more and 6 or less carbon atoms;
R^{a3} is a methylene group, —O—, —CO—, —CO—O—, —SO—, —SO₂—, or —NR^{a6}—;
R^{a6} is a hydrogen atom or a hydrocarbon group having 1 or more and 6 or less carbon atoms;
R^{a4} is an optionally substituted aromatic group having 5 or more and 20 or less ring constituting atoms, a perfluoroalkyl group having 1 or more and 6 or less carbon atoms, an optionally substituted aralkyl group having 7 or more and 20 or less carbon atoms, or a heteroarylalkyl group having an optionally substituted aromatic heterocyclic group having 5 or more and 20 or less ring constituting atoms;
Q¹ and Q² are each independently a fluorine atom or a perfluoroalkyl group having 1 or more and 6 or less carbon atoms; and
L is an ester bond.

10. The compound according to claim 9, wherein the compound represented by the formula (A1) is a compound represented by the following formula (A1-1): 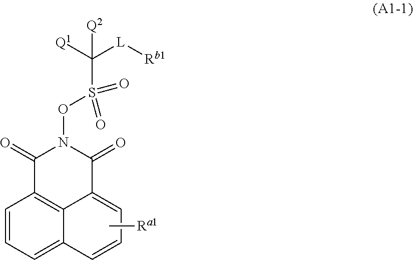
wherein R^{b1}, R^{a1}, Q¹, and Q² are the same as those in the formula (A1).

11. The compound according to claim 10,
wherein R^{a1} is an optionally substituted aliphatic hydrocarbon group having 1 or more and 20 or less carbon atoms, and
when the aliphatic hydrocarbon group as R^{a1} contains 1 or more methylene groups, at least a part of the methylene groups may be replaced with a group selected from the group consisting of —O—, —S—, —CO—, —CO—O—, —SO—, —SO₂—, and —NR^{a5}—.

12. A photoacid generator, comprising the compound according to claim 9.

13. A method of manufacturing a N-organosulfonyloxy compound,
the method comprising: reacting a N-hydroxy compound (A′) and a sulfonyl fluoride compound (B′) in the presence of a basic compound (D′),
wherein a silylating agent (C′) is present in the system while the N-hydroxy compound (A′) and the sulfonyl fluoride compound (B′) are reacted,
the sulfonyl fluoride compound (B′) is represented by the following formula (bi1):
R^{bi1}—SO₂—F (bi1)
wherein R^{bi1} is an organic group, and
the silylating agent (C′) is capable of converting a hydroxy group on the nitrogen atom possessed by the N-hydroxy compound (A′) into a silyloxy group represented by the following formula (ac1):
—O—Si(R^{c1})₃ (ac1)
wherein each R^{c1} is independently a hydrocarbon group having 1 or more and 10 or less carbon atoms.

14. A method of manufacturing a N-organosulfonyloxy compound,
the method comprising: silylating a N-hydroxy compound (A′) with a silylating agent (C′), and
condensing a silylated product of the N-hydroxy compound (A′) generated in the silylation step with a sulfonyl fluoride compound (B′) in the presence of a basic compound (D′),
wherein the sulfonyl fluoride compound (B′) is represented by the following formula (bi1):
R^{bi1}—SO₂—F (bi1)
wherein R^{bi1} is an organic group, and
the silylating agent is capable of converting a hydroxy group on the nitrogen atom possessed by the N-hydroxy compound (A′) into a silyloxy group represented by the following formula (ac1):
—O—Si(R^{c1})₃ (ac1)
wherein each R^{c1} is independently a hydrocarbon group having 1 or more and 10 or less carbon atoms.

15. The method of manufacturing a N-organosulfonyloxy compound according to claim 13, wherein one or two carbonyl groups are present at a position or positions adjacent to a nitrogen atom to which the hydroxy group is bonded in the N-hydroxy compound (A′).

16. The method of manufacturing an N-organosulfonyloxy compound according to claim 13,
wherein the sulfonyl fluoride compound (B′) is represented by the following formula (bi1-1):
R^{bi2}—CQ¹Q²-SO₂—F (bi1-1)
wherein Q¹ and Q² are each independently a fluorine atom or a perfluoroalkyl group having 1 or more and 6 or less carbon atoms, and R^{bi2} is an organic group.

17. The method of manufacturing an N-organosulfonyloxy compound according to claim 16,
wherein the sulfonyl fluoride compound (B′) is represented by the following formula (bi1-2):
R^{bi3}-Lⁱ-CQ¹Q²-SO₂—F (bi1-2)
wherein Q¹ and Q² are the same as those in the formula (bi1-1);
L′ is —CO—O— or —O—;
R^{bi3} is a hydrocarbon group having 1 or more and 30 or less carbon atoms;
when the hydrocarbon group as R^{bi3} contains 1 or more methylene groups, at least a part of the methylene groups may be replaced with a group selected from the group consisting of —O—, —S—, —CO—, —CO—O—, —SO—, —SO₂—, —CR^{b4}R^{b5}—, and —NR^{b6}—;
when the hydrocarbon group as R^{bi3} contains a hydrocarbon ring, at least one of the carbon atoms constituting the hydrocarbon ring may be replaced with a heteroatom selected from the group consisting of N, O, P, S, and Se or an atomic group containing the heteroatom;
R^{b4} and R^{b5} are each independently a hydrogen atom or a halogen atom, and at least one of R^{b4} and R^{b5} is a halogen atom; and
R^{b6} is a hydrogen atom or a hydrocarbon group having 1 or more and 6 or less carbon atoms.

18. The method of manufacturing an N-organosulfonyloxy compound according to claim 13, wherein the N-hydroxy compound (A′) is a N-hydroxyimide compound.

19. The method of manufacturing an N-organosulfonyloxy compound according to claim 18, wherein the N-hydroxyimide compound is a N-hydroxy aromatic dicarboxylic acid imide compound.

20. The method of manufacturing an N-organosulfonyloxy compound according to claim 19, wherein the N-hydroxy aromatic dicarboxylic acid imide compound is a N-hydroxy naphthalimide compound which may have at least one substituent on the naphthalene ring.
